SEQUENCE LISTING (73 sequences)

SEQ ID NO: 1 - DNA, 5006 bp, Artificial Sequence
  Synthetic NGGT0009 plasmid with CAG promoter/SV40 polyA for expressing transgene
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccggtaccaccggtgtcgacaagcttcccttaattaaccgtttaaacaattctgcaggaatctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgatttgataccgcgggcccatcgctagcatggatcccctgcaggacgcgttcgcgagaattctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatttaaattgggcgcgcccaactagttctagagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 3171 bp, Artificial Sequence
  Synthetic NGGT0002 plasmid with human U6 promoter for expressing siRNAs
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggcgcgccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgtcgacaagcttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 3 - DNA, 1503 bp, Artificial Sequence
  Synthetic WT human SERPING1 cDNA
atggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctga

SEQ ID NO: 4 - DNA, 1503 bp, Artificial Sequence
  Synthetic Codon optimized human SERPING1 cDNA GW-50
atggcctctagactgaccctgctgaccctcctgttgctgttgctggctggggacagagctagcagcaaccccaatgccacaagcagcagcagccaagaccctgagagcctgcaagacagaggggagggcaaggtggccaccacagtgatcagcaagatgctgtttgtggagcccatcctggaggtgagcagcctgcccaccaccaactccaccaccaactctgccaccaagatcactgccaataccacagatgagcccaccacccaacctaccacagagcctaccacacagcccaccattcagcccacacagcccaccacacaactgcccacagacagccctacacagcctaccactggcagcttctgccctggccctgtgaccctgtgctctgacctggagagccacagcacagaggcagtgctgggggatgccctggtggacttcagcctgaagctgtaccatgccttctctgccatgaagaaggtggagaccaacatggccttcagccccttcagcattgccagcctgctgacccaagtgctgctgggcgccggcgagaacaccaagaccaacctggagagcatcctgtcctaccccaaggacttcacatgtgtgcaccaagccctgaagggctttaccaccaagggggtgacatctgtgtctcagatcttccacagccctgacctggccatcagagacacctttgtgaatgccagcagaaccttgtacagctctagccctagagtgctgtccaacaactctgatgccaacctggagctgatcaacacctgggtggccaagaacaccaacaacaagattagcagattgctggacagcctgccttctgacacaagactggtgctgctgaatgccatctacctgtctgccaagtggaagaccacttttgacccaaaaaaaacaagaatggagcccttccacttcaagaacagcgtgatcaaagtgcccatgatgaacagcaagaagtaccctgtggcccacttcattgaccaaaccctgaaggccaaggtggggcagctgcagctgagccacaacctgagcctggtgatcctggtgcctcagaacctgaagcacagactggaggacatggagcaagccctgagcccctctgtgttcaaggctatcatggaaaagctggagatgtccaagtttcagcccacactgctgacactgcctagaatcaaggtgaccacaagccaagacatgctgagcattatggagaagctggagttctttgacttcagctatgacctgaacctgtgtggcctgacagaggaccctgacctgcaagtttctgccatgcagcatcagacagtgctggagctgacagagactggggtggaggctgctgctgcctctgccatctctgtggctagaaccctgctggtgtttgaggtgcagcagcccttcctgtttgtgctgtgggatcagcagcacaagttccctgtcttcatgggcagagtgtatgaccctagagcctga

SEQ ID NO: 5 - DNA, 1503 bp, Artificial Sequence
  Synthetic Codon optimized human SERPING1 cDNA GS-26
atggccagcagactgaccctgttgacactgctgctgctgctgctggctggagacagagccagcagcaaccctaatgccaccagcagcagctcccaggaccctgaaagcttacaggacagaggcgagggcaaagtggccacaacagtgatcagcaagatgctgttcgtggaacctatcctggaggtgagcagcctgcccacaaccaacagcaccacaaactctgccaccaagatcacagccaacaccacagatgaacctaccacccagcccaccacagaacctaccacccagcctaccatccagcccacccagcctacaacccaactgcctacagacagccccacacagcctaccactggcagcttctgcccaggccctgtgaccctgtgctccgacctggagtctcacagcacagaagctgtgctgggcgatgccctagtggatttcagcctgaagctgtaccatgccttctctgccatgaaaaaagtggagaccaacatggccttctcccccttcagcattgcttccctgctgacccaggtgctgctgggcgctggagaaaataccaagaccaatctcgagagcatcctgagctaccctaaagacttcacctgtgtgcaccaggccctgaagggcttcaccacaaagggcgtgacatctgtcagccaaatcttccacagcccagatctggccatccgggacacctttgtgaacgcctcccggaccctgtacagcagcagcccacgggtactgagcaacaattctgatgccaacctagagctgatcaacacctgggtggccaagaacaccaacaacaagatctccagactgctggacagcctgccctctgacaccagactggtgctgttaaatgccatctacctgtcagccaagtggaagaccacctttgatcccaaaaagacacggatggagcccttccacttcaagaactctgtgatcaaggtgcctatgatgaacagcaagaagtaccctgtggcacacttcatcgaccagacactgaaagccaaagtgggccagctgcagctgagccacaacctgtccctggtcattctggtgccacagaaccttaagcacagactggaagacatggagcaagccctgagcccttcagtgttcaaggccatcatggaaaagctggagatgtctaagttccaacctacactgctgaccctgcctaggatcaaggtgaccacaagccaggacatgctgagcatcatggagaagctggaattctttgacttctcttacgatctgaacctgtgcggcctgacagaggaccctgacctgcaggtctctgccatgcagcaccagactgtgctggagctgactgaaacaggagtggaggctgcagctgccagtgccatctcagtggccagaaccctcctggtgtttgaggtgcaacagccattcctgtttgtcctgtgggaccagcagcacaagttccctgttttcatgggcagagtctatgatcccagagcctga

SEQ ID NO: 6 - DNA, 1503 bp, Artificial Sequence
  Synthetic Codon optimized human SERPING1 cDNA GS-6
atggccagcaggctgaccctgctgaccctgctgctgctgctcctggctggagacagagccagcagcaacccaaatgccaccagcagcagcagccaagaccctgaatccctgcaggacagaggagaaggcaaggtggccacaacagtgatcagcaagatgctgtttgtggagcctatcctggaagtcagcagcctgcctaccaccaacagcaccaccaactctgccaccaagatcacagccaacaccactgatgagcctaccacccagcctaccacagagcccaccacccagcccaccatccagcccacacagcctaccacacagctgccaacagacagccctacccagcccacaacaggctccttctgccctggccctgtgaccctttgttctgatctggagtcccactctacagaggctgtgctgggtgatgccctggtggacttcagcctgaagctgtaccatgccttctctgccatgaaaaaggtggagaccaacatggccttcagccctttcagcattgcctccctgctcacccaggtgctgctgggcgctggagaaaacaccaagacaaacctggaaagcatcctgagctaccccaaggacttcacctgcgtgcaccaggccctgaaaggcttcaccacaaagggcgtgacctctgtgagccagatcttccacagcccagacctggccatcagagacacctttgtgaatgcctcccggaccctgtacagcagcagccccagagtgctgagcaacaactctgatgccaacctggagctgatcaacacatgggtggctaagaacaccaacaacaagatcagcagactgctggatagcctgccctctgacacaagactggtcctgctgaatgccatctatctgtctgccaagtggaaaacaacctttgaccctaagaagacccggatggagcctttccatttcaagaacagcgtgatcaaggttcctatgatgaattccaaaaagtaccctgtggcccacttcattgatcaaacccttaaggccaaagtgggccagctccagctgagccacaacctgagcctggtgatcctggtgcctcagaatctgaagcatagactggaagacatggagcaggccctgagcccttctgtgttcaaggccatcatggaaaagctggagatgagcaagttccagcctaccctgctgaccctgcccagaatcaaagtcaccaccagccaggacatgctgagcatcatggagaagctggaattctttgacttctcctatgacctgaacctgtgtggcctgacagaggatcctgacctgcaggtttctgccatgcagcaccagacagtgctggagctgacagaaacaggagtggaggctgctgctgccagtgccatctctgtggccagaaccctgctggtgtttgaggtgcaacagcccttcctgtttgtgctgtgggaccagcagcacaagttcccagtgttcatgggcagagtgtatgatcctagggcctaa

SEQ ID NO: 7 - DNA, 1503 bp, Artificial Sequence
  Synthetic Codon optimized human SERPING1 cDNA GS-2
atggcaagcaggctgaccctcctgaccctgctgctgctgctgctggctggagacagagccagcagcaaccctaatgccaccagcagctctagccaggaccctgagagcctgcaggacagaggagagggcaaagtggccaccactgtgatctcaaagatgctgtttgtggaaccaatcctggaggtaagcagcctgcctacaacaaacagcaccaccaactctgccaccaaaatcacagccaacaccacagatgaacctacaacccagcctaccacagaacccaccacccagcctaccatccagcccacacagcccacaacacaactgcctacagatagccctacacagcctaccacaggcagcttctgcccaggcccagtgaccctgtgcagtgacttagagagccactccacagaagcagtcctgggagatgccctggtggacttcagcctgaagctctaccatgccttcagtgccatgaagaaggtggagactaacatggccttcagccctttctccattgccagcttgctgacccaggtgctgctgggggctggtgagaacaccaagacaaacctggaaagcatcctgagctaccctaaggacttcacctgtgttcaccaggctctgaagggcttcacaaccaagggggtgacctctgtgagccagatcttccactcccctgacctggccatcagggacacctttgtgaatgccagcagaaccctgtacagcagcagccctagagtgctgagtaacaactctgatgccaaccttgaactgatcaacacctgggtggccaagaacaccaacaataagatctctagactgctggacagcctgccctctgacaccagacttgtcctgctgaatgccatctacctgtcagccaagtggaagaccacctttgaccccaagaagaccagaatggagcccttccacttcaagaactctgtgatcaaagtgcccatgatgaactccaagaagtacccagtggcccacttcattgatcagaccctgaaagccaaggtgggccaactgcagctgagccacaacttgagcctggtgatcctggtgccccagaacctgaaacacaggctggaggacatggagcaagctctgagcccttctgtgttcaaggccatcatggagaagctggagatgagcaagttccaacctaccctgctgaccctgcccagaatcaaagtgacaaccagccaggatatgctgtcaatcatggaaaagctggagttctttgacttcagctatgatctgaatctgtgtggcctgacagaggaccctgacctgcaggtgtctgccatgcagcaccagacagtccttgaactgacagaaacaggagtggaggctgctgctgcctctgccatctctgtggccagaaccctcctggtgtttgaggtgcagcagcctttcctgtttgtgctgtgggaccagcagcacaagttccctgtgttcatgggcagagtgtatgatccaagagcctaa

SEQ ID NO: 8 - DNA, 1503 bp, Artificial Sequence
  Synthetic Codon optimized human SERPING1 cDNA GS-8
atggccagcagactgaccctgctgaccctgctgctgcttctgctggctggtgacagagccagcagcaaccctaatgccaccagcagctccagccaggacccagagtccctccaggataggggagagggcaaagtggccaccacagtgatcagcaagatgctgtttgtggagcccatcctggaggtcagctctctgcctaccaccaactccaccaccaactctgccaccaagatcacagccaacacaacagatgagcccacaacccagcctaccacagagcctaccacccaacctaccatccagcctacccagcccactacccagctgcctacagacagccctacccagcccacaacaggcagcttctgccctggcccagtgaccctgtgttctgacctggaaagccacagcacagaagctgtgctgggagatgccctggtggacttcagcctgaagctgtaccatgccttctctgccatgaagaaggtggaaaccaacatggccttcagccctttctccattgccagcctgctgacccaggtgctgctgggcgctggagagaacaccaagacaaacctggagagcatcctgagctaccccaaggacttcacatgcgtccatcaggccctgaaaggcttcacaaccaagggcgtgacctctgtgagccagatcttccacagccctgacctggccatccgggacacatttgtgaatgcttccagaaccctgtacagcagcagccctagagtgctgagcaacaacagcgatgccaacctggaactgatcaacacctgggtggccaagaacaccaacaacaagatcagcagactgctggactccctgccctctgacaccagactggttctcctgaatgccatctatctgtctgccaagtggaagaccacatttgatcccaaaaagacccggatggaaccattccacttcaagaactctgtgatcaaagtgcctatgatgaatagcaagaagtaccctgtggcccacttcattgaccagaccctgaaggccaaggtgggccagctgcagctgagccataacctcagcctggtgatcctggtgcctcagaatctgaagcacagactggaggacatggagcaggccctgtcccctagtgtgttcaaggccatcatggaaaaactggagatgagcaagttccagcccaccctgctgaccctgcccagaatcaaggtgaccaccagccaggacatgctgagcatcatggaaaaacttgagttctttgatttcagctatgatctgaacctgtgtggcctgacagaggaccctgacctgcaagtgtctgccatgcagcaccaaacagtgctggaactgacagaaacaggagttgaggctgctgctgcctctgccatctctgtggccagaaccctgctggtgtttgaggtgcagcagcctttcctgtttgtcctgtgggaccagcagcacaagttcccagtgttcatgggcagagtgtatgatcctagggcctaa

SEQ ID NO: 9 - DNA, 1503 bp, Artificial Sequence
  Synthetic Codon optimized human SERPING1 cDNA GS-15
atggcctccagactgaccctcctgaccctgctgctgctgctgctggctggagacagagccagcagcaaccccaatgccaccagcagcagcagccaggatcctgagagcctgcaggacaggggtgagggcaaggtggccacaacagtcatcagcaagatgctgtttgtggaacccatcctggaagtgtcctccctgcccaccacaaacagcacaaccaactctgccaccaagatcacagccaacaccacagatgagcccaccacccagccaaccactgagcctaccacccaaccaaccatccagcctacccagcctaccacccagctgcctacagacagccctacacagcctaccacaggcagcttctgccctggccctgtgaccctgtgttctgacctggagagccactctacagaggctgtgctgggagatgccctggtggacttcagcctgaagctgtaccatgccttctctgccatgaagaaagtggaaaccaacatggccttcagccccttctccattgccagcctcctgacccaggtgctgctgggagctggcgagaacaccaagacaaacctggagagcatcctgagctaccccaaggacttcacctgcgtgcaccaggccctgaagggcttcaccaccaaaggcgtgacatctgtgtcccagatcttccacagcccagacctggccatcagagacaccttcgtgaatgcttccagaacactgtacagcagcagccctagagtgctgagcaacaactctgatgccaacctggaactgatcaacacctgggttgccaaaaataccaacaacaagatcagcagactgctggacagcctgcctagcgacacacggctggtcctgctgaatgccatctacctgtctgccaagtggaagaccacctttgatcctaagaaaacacggatggaaccttttcacttcaagaactctgtgatcaaggtgcccatgatgaacagcaagaagtatcctgtggcccatttcattgaccagaccctgaaagccaaagtgggccagctgcagctgagccacaacctgagcctggtgatcctggtgcctcagaatctgaagcacagactggaggacatggagcaggccctgtccccatctgtgttcaaggccatcatggaaaagcttgagatgagcaagttccagcctaccctgctgaccctgcctaggatcaaggtgaccaccagccaggacatgctgagcatcatggagaagctggagttctttgacttcagctatgatctgaacctgtgtggcctgacagaagatcctgacctgcaagtgtctgccatgcagcatcagacagttctggaactgacagagacaggagtggaagctgctgctgcctctgccatcagtgtggccagaaccctcctggtctttgaggtgcagcaacccttcctgtttgtgctgtgggatcagcagcacaagttccctgtgttcatgggcagagtgtatgaccccagagcctaa

SEQ ID NO: 10 - DNA, 1503 bp, Artificial Sequence
  Synthetic Codon optimized human SERPING1 cDNA GS-16
atggccagcagactgacccttctgaccctgttgttgctgctgctggctggtgatagagccagcagcaatcccaatgccaccagcagcagcagccaagatcctgagagcctgcaagacaggggagagggcaaagtggccacaacagtgatcagcaagatgctgtttgtggaacctatcctggaggtgtccagcctgcctaccaccaactccactaccaactctgccaccaagatcacagcaaacaccacagatgagcccacaacccagcctacaacagagcccacaacacaacctactatccagcccacccagccaaccacccagctgcctacagacagtcctacccagcctaccacaggcagcttctgcccaggcccagtgaccctctgctctgacctggaatcccacagcacagaagctgtcctaggagatgccctggtggacttcagcctgaagctgtaccatgccttctctgccatgaaaaaagtggaaaccaacatggccttcagccctttcagcattgccagcctgctgacccaggtgctgctgggagctggggaaaacaccaagactaacctggagtcaatcctgagctaccctaaggacttcacctgtgttcaccaagccctgaaaggcttcaccacaaaaggagtgacatctgtgagccagatcttccacagccctgatctggccatcagagacacctttgtcaatgcctctaggaccctgtacagctccagcccaagagtgctgagcaacaactctgatgccaacctggagctgatcaacacctgggtggccaagaacaccaacaacaagatcagcagactcctggacagcctgccttcagacaccagattagtgctcctgaatgccatctacctatctgccaagtggaaaaccacctttgaccccaagaagaccagaatggagcctttccacttcaagaactctgtcatcaaggtgcctatgatgaacagcaagaagtacccagttgcccacttcattgaccagaccctcaaggccaaagtgggccagctgcagctgagccacaacctgagcctggtcatcctggtgccccagaacctgaagcacagattagaagacatggaacaggccctgagcccttctgtgttcaaggccatcatggagaagctggagatgtccaagttccagcccaccctgcttacactgcctagaatcaaggtgacaaccagccaggacatgctgtcaatcatggagaagctggaattctttgacttcagctatgacctgaatctgtgtggcctgacagaggaccctgacctgcaggtgtctgccatgcagcaccagacagtgctggagctcacagagacaggagtggaagctgctgctgcctctgctatctctgtggccagaacactgctggtgtttgaggtgcagcagcccttcctgtttgtgctgtgggaccagcagcacaagttccctgtgttcatgggcagggtgtatgatcctagagcctga

SEQ ID NO: 11 - DNA, 1503 bp, Artificial Sequence
  Synthetic Codon optimized human SERPING1 cDNA GS-19
atggccagcagactgaccctgctgacactgctactgctgctgctggctggagacagagccagcagcaaccctaatgccacctctagcagctcccaagaccctgagagcctgcaggacagaggtgaaggcaaggttgccactactgtgatcagcaagatgctatttgtggagcctatcctggaggtgagcagcctgcccacaaccaacagcaccaccaactctgccacaaagatcacagccaacaccacagatgaacctacaacacagcccaccacagagcctaccacacagcccaccatccagcctacccagcctaccacacaactgcctacagattcccctactcagcctacaacaggcagcttctgccctggccctgtgaccctgtgcagtgatctggagagccacagcacagaggctgtgttgggagatgccctggtggacttctctctgaagctgtaccatgccttctctgccatgaagaaggtggagaccaacatggccttctctcctttcagtattgccagcctgttaacacaggtgctgctgggggctggggagaacaccaagaccaacctggagagcatcctgagctaccctaaggacttcacctgcgtccaccaggccctcaagggcttcacaaccaaaggagtgaccagtgtgagccagattttccactccccagacctggccatcagggacacctttgtcaatgccagcaggaccctgtacagcagcagccccagagtgctgagcaacaactcagatgccaacctggaactcatcaacacctgggtggccaaaaacaccaacaacaagatcagcagactgctggacagcctgccttcagataccagactggtgctcctgaatgccatctacctgtctgccaagtggaagaccacctttgaccctaagaagaccagaatggagcctttccacttcaagaattctgtgatcaaggtgcccatgatgaacagcaaaaagtacccagtggcccacttcatagatcagaccctgaaggccaaggttggccagctgcagctgtctcacaacctgagcctggtgatcctggtgccccagaatctgaagcacagactggaagacatggagcaggctctgtctccttctgtgttcaaggccatcatggagaagctggaaatgagcaaattccagcccacactgttgaccctgcctagaatcaaggtgaccacctcccaggacatgctgtccatcatggagaaactggagttctttgacttcagctatgaccttaacctgtgtggcctgacagaagacccagacctgcaggtgtctgccatgcagcaccagacagtcctggaactgactgaaacaggagttgaagctgctgctgctagtgccatctcagtggccagaaccctgctggtgtttgaggtgcagcagcccttcctgtttgtgctgtgggaccaacagcacaaattccctgtgttcatgggcagagtgtatgatcccagggcctga

SEQ ID NO: 12 - DNA, 1503 bp, Artificial Sequence
  Synthetic Codon optimized human SERPING1 cDNA GS-41
atggcatccagactcaccctcctcactcttcttttactcctcctggctggagacagggcttcctccaaccctaatgccactagctccagcagccaggacccagagagcctgcaggacagaggagagggaaaagtggccaccactgtgatctccaagatgctgtttgtggagcctatcctggaagtgtccagccttcccaccactaactccaccaccaactcagccaccaagatcactgccaacaccactgatgagcctaccactcaacccaccactgagcccactacccaacccacaattcagcccacccagcctaccacccaactgccaactgatagccctacccagcccaccactgggtccttctgccctggccctgtgaccctgtgcagtgacctggaatcccactccactgaagctgtgttgggagatgccctggtggacttcagcctgaagctctaccatgcattctcagccatgaagaaagtggaaaccaacatggccttctcccccttctccattgcctccctgctcacccaagtcctgctgggtgcaggggaaaacaccaagaccaacctggagagcatcctgagctaccccaaggacttcacctgtgtgcatcaggccctcaagggtttcacaaccaagggagtcacctcagtgtcccaaatcttccattcccctgacctggccattagagacacctttgtgaatgcctcccgcactctgtactcctcctccccacgggtgctgagcaacaactcagatgccaaccttgagctgatcaacacctgggtggccaagaacaccaacaacaagattagccgcctgctggatagcctgccctcggacactcggctggtgctcctgaatgccatctacctgtctgccaagtggaaaaccacctttgaccccaaaaagacccggatggaaccgttccacttcaagaactcagtgattaaggtccccatgatgaacagcaagaagtacccggtggcccacttcattgaccagaccctcaaggccaaagttggccagctgcagttgtcccacaacttgtccctggtcatcctggtgccccaaaacttgaagcacaggcttgaggacatggaacaggccctgtccccttctgtgttcaaggccatcatggaaaagctggagatgtccaagttccagcctaccctgctgaccctgccaagaatcaaggtcacaacctcccaagacatgctgtccatcatggagaagctggaattctttgacttctcctatgacctgaacctctgtggcctgactgaagatccagacctccaagtgtcagccatgcagcaccagactgtgttggagctaactgagactggggtggaagctgcagctgcctcagccatctcagtggccaggaccctcctggtctttgaagtgcagcagcccttcctctttgtcctgtgggatcagcagcacaagttccctgtgttcatgggcagggtctatgacccaagagcataa

SEQ ID NO: 13 - DNA, 1503 bp, Artificial Sequence
  Synthetic Codon optimized human SERPING1 cDNA GS-47
atggccagcagactgaccctcctgacactgctgctgcttctgctggctggagacagagcctctagcaaccccaatgccacaagcagcagcagccaggatcctgagagcctgcaggacaggggtgagggcaaggtggccaccactgtcatcagcaagatgctgtttgtggaacctatcctggaagtgtcctccctgccaaccaccaacagcaccaccaactctgccaccaagatcacagccaacaccacagatgagcctaccacacagcccaccactgagcccaccacccagcctaccatccagcctacacagcctaccacccagctgcctacagacagccccacccaaccaaccacaggcagcttctgccctggccctgtgaccctgtgttctgacctggaatcccacagcacagaggctgtgctgggagatgccctggtggacttcagcctgaagctgtaccatgccttctctgccatgaagaaagtggagacaaacatggccttttctccattcagcatcgcctctctgctgacccaggtgctgctgggcgccggcgagaacaccaagaccaatctcgagagcatcctgtcctatcctaaggacttcacctgcgtgcaccaggccctgaaaggatttacaacaaagggcgtgacatccgtcagccagatcttccacagccctgatctggccattagagataccttcgtgaacgcttctagaaccctgtacagcagctctcctagagtgctgagcaacaattccgacgccaacctggaactgatcaacacatgggtggctaaaaataccaacaacaagatcagccggctgctggacagcctgcctagcgacaccagactggtgctgctgaacgccatctacctgagcgccaagtggaagaccacattcgaccccaagaaaacccggatggaacctttccacttcaagaacagcgtgatcaaggtgcccatgatgaacagcaagaagtaccccgtggcccatttcattgaccagaccctcaaagccaaagtgggccagctgcagctcagccacaacctgagcctggtgatcctggttcctcagaatctgaagcacagactggaggacatggaacaggccctgtccccatctgtgttcaaggccatcatggaaaagctggagatgagcaagttccagcccaccctgctgaccctgcctaggatcaaggtgaccaccagccaggacatgctgagcatcatggagaagctggagttctttgacttcagctatgatctgaacctgtgtggcctgactgaggatcctgacctgcaagtgtctgccatgcagcaccagacagtgctggagctgacagagacaggagtggaagctgctgctgcctctgccatcagtgtggccagaacactgctggtctttgaagtgcagcagcctttcctgtttgtgctgtgggatcagcaacataagttccctgtgttcatgggcagagtgtatgaccccagagcctaa

SEQ ID NO: 14 - DNA, 1503 bp, Artificial Sequence
  Synthetic Codon optimized human SERPING1 cDNA GS-48
atggccagcagactgaccctgctgacactgctcctgctgctgctggctggtgacagagcctctagcaaccccaatgccaccagctccagcagccaggaccctgagagcctgcaggataggggagagggcaaggtggccacaacagtgatcagcaagatgctgtttgttgagcccatcctggaagtgtcctccctgcctacaaccaacagcaccaccaactctgctaccaagatcacagccaacaccacagatgagccaaccacccagcctaccactgaacctaccacccagcctaccatccagcctacccaaccaaccacccagctgcccacagacagccctacccagcccaccacaggcagcttctgccctggccctgtgaccctgtgttctgacctggaaagccacagcacagaggctgtcctgggagatgccctggtggacttcagcctgaagctgtaccatgccttctctgccatgaagaaagtggagacaaacatggccttcagcccattttccatcgccagcctgctgacccaggtgctcctgggcgccggcgagaacaccaagaccaacctggagagcatcctgagctaccccaaggacttcacctgcgtgcaccaggccctgaagggcttcaccacaaaaggagtgaccagcgtgtcccagatcttccacagccccgacctggccattagagatacattcgtgaacgcttctagaaccctgtacagcagcagccctagagtgctgagcaacaactccgatgccaatctggaactgatcaacacctgggtcgccaaaaatacaaacaacaagatcagccggctgctggactctctgcctagcgacaccagactggtgctgctgaacgccatctacctgtctgccaagtggaagacaaccttcgaccctaagaaaacacggatggaaccttttcacttcaagaattctgttatcaaggtgcccatgatgaacagcaagaagtatcctgtggctcatttcattgatcagaccctcaaagccaaagtgggccagctgcagctgagccacaacctgagcctggtgatcctggtgcctcagaatctgaagcacagactggaggacatggaacaggccctgtccccaagtgtgttcaaggccatcatggaaaagctggagatgagcaagttccagcccaccctgctgaccctgcctaggatcaaggtgaccaccagccaggacatgctgagcatcatggagaagctggagttctttgacttcagctatgatctgaacctgtgtggcctgacagaggatcctgacctgcaagtgtctgccatgcagcaccagacagttctggagctgacagagacaggagtggaagctgctgctgcctctgccatctctgtggccagaacccttctggtctttgaagtgcagcagcctttcctgtttgtgctgtgggaccagcaacataagttccctgtgttcatgggcagagtgtatgaccccagagcctaa

SEQ ID NO: 15 - DNA, 19 bp, Artificial Sequence
  Synthetic Human SERPING1 siRNA Homo-37
gcaagggacctgcttctat

SEQ ID NO: 16 - DNA, 19 bp, Artificial Sequence
  Synthetic Human SERPING1 siRNA Homo-44
gttcaccagactctataaa

SEQ ID NO: 17 - DNA, 19 bp, Artificial Sequence
  Synthetic Human SERPING1 siRNA Homo-3
caaccaactcaacaaccaa

SEQ ID NO: 18 - DNA, 19 bp, Artificial Sequence
  Synthetic Human SERPING1 siRNA Homo-7
gttactctctgctctgact

SEQ ID NO: 19 - DNA, 19 bp, Artificial Sequence
  Synthetic Human SERPING1 siRNA Homo-6
ccaagatgctattcgttga

SEQ ID NO: 20 - DNA, 19 bp, Artificial Sequence
  Synthetic Human SERPING1 siRNA Homo-33
cagtctctcagatcttcca

SEQ ID NO: 21 - DNA, 19 bp, Artificial Sequence
  Synthetic Human SERPING1 siRNA Homo-14
ctccaagatgctattcgtt

SEQ ID NO: 22 - DNA, 19 bp, Artificial Sequence
  Synthetic Human SERPING1 siRNA Homo-10
gaaggtcgcaacaacagtt

SEQ ID NO: 23 - DNA, 53 bp, Artificial Sequence
  Synthetic Human SERPING1 shRNA Homo-37
gcaagggacctgcttctatttcaagagaatagaagcaggtcccttgctttttt

SEQ ID NO: 24 - DNA, 53 bp, Artificial Sequence
  Synthetic Human SERPING1 shRNA Homo-44
gttcaccagactctataaattcaagagatttatagagtctggtgaactttttt

SEQ ID NO: 25 - DNA, 54 bp, Artificial Sequence
  Synthetic Human SERPING1 shRNA Homo-3
gcaaccaactcaacaaccaattcaagagattggttgttgagttggttgtttttt

SEQ ID NO: 26 - DNA, 53 bp, Artificial Sequence
  Synthetic Human SERPING1 shRNA Homo-7
gttactctctgctctgactttcaagagaagtcagagcagagagtaactttttt

SEQ ID NO: 27 - DNA, 54 bp, Artificial Sequence
  Synthetic Human SERPING1 shRNA Homo-6
gccaagatgctattcgttgattcaagagatcaacgaatagcatcttggtttttt

SEQ ID NO: 28 - DNA, 54 bp, Artificial Sequence
  Synthetic Human SERPING1 shRNA Homo-33
gcagtctctcagatcttccattcaagagatggaagatctgagagactgtttttt

SEQ ID NO: 29 - DNA, 54 bp, Artificial Sequence
  Synthetic Human SERPING1 shRNA Homo-14
gctccaagatgctattcgttagagaacttaacgaatagcatcttggagtttttt

SEQ ID NO: 30 - DNA, 53 bp, Artificial Sequence
  Synthetic Human SERPING1 shRNA Homo-10
gaaggtcgcaacaacagttagagaacttaactgttgttgcgaccttctttttt

SEQ ID NO: 31 - DNA, 58 bp, Artificial Sequence
  Synthetic Human SERPING1 3UTR _03486
ccggccatgctctccaaaccactttctcgagaaagtggtttggagagcatggtttttg

SEQ ID NO: 32 - DNA, 59 bp, Artificial Sequence
  Synthetic Human SERPING1 shRNA 3UTR1
ccggggcaagggacctgcttctatttcaagagaatagaagcaggtcccttgcctttttg

SEQ ID NO: 33 - DNA, 59 bp, Artificial Sequence
  Synthetic Human SERPING1 shRNA 3UTR2
ccgggcaagggacctgcttctattatcaagagtaatagaagcaggtcccttgctttttg

SEQ ID NO: 34 - DNA, 59 bp, Artificial Sequence
  Synthetic Human SERPING1 shRNA 3UTR3
ccgggcagctttctctagttcaagttcaagagacttgaactagagaaagctgctttttg

SEQ ID NO: 35 - DNA, 59 bp, Artificial Sequence
  Synthetic Human SERPING1 shRNA 3UTR4
ccgggctttctctagttcaagttcatcaagagtgaacttgaactagagaaagctttttg

SEQ ID NO: 36 - DNA, 59 bp, Artificial Sequence
  Synthetic Human SERPING1 shRNA 3UTR5
ccgggttcaagttcaccagactctatcaagagtagagtctggtgaacttgaactttttg

SEQ ID NO: 37 - DNA, 41 bp, Artificial Sequence
  Synthetic Human SERPING1 shRNA 3UTR6
ccgggttcaccagactctataaatatcaagagtatttatag

SEQ ID NO: 38 - DNA, 58 bp, Artificial Sequence
  Synthetic shRNA_SERPING1_73515
ccgggggatagagcctcctcaaatcctcgaggatttgaggaggctctatccctttttg

SEQ ID NO: 39 - DNA, 58 bp, Artificial Sequence
  Synthetic Human SERPING1 shRNA 73451
ccggagttatctccaagatgctattctcgagaatagcatcttggagataacttttttg

SEQ ID NO: 40 - DNA, 19 bp, Artificial Sequence
  Synthetic Mouse Serping1 siRNA Mus-4
cttcatgggtcgtgtatat

SEQ ID NO: 41 - DNA, 19 bp, Artificial Sequence
  Synthetic Mouse Serping1 siRNA Mus-6
gctgagaacaccaaccata

SEQ ID NO: 42 - DNA, 19 bp, Artificial Sequence
  Synthetic Mouse Serping1 siRNA Mus-8
ccaattcgatgaccatact

SEQ ID NO: 43 - DNA, 19 bp, Artificial Sequence
  Synthetic Mouse Serping1 siRNA Mus-10
cagtgtagccatcacaata

SEQ ID NO: 44 - DNA, 54 bp, Artificial Sequence
  Synthetic Mouse Serping1 shRNA-4
gcttcatgggtcgtgtatatttcaagagaatatacacgacccatgaagtttttt

SEQ ID NO: 45 - DNA, 53 bp, Artificial Sequence
  Synthetic Mouse Serping1 shRNA-6
gctgagaacaccaaccatattcaagagatatggttggtgttctcagctttttt

SEQ ID NO: 46 - DNA, 54 bp, Artificial Sequence
  Synthetic Mouse Serping1 shRNA-8
gccaattcgatgaccatactttcaagagaagtatggtcatcgaattggtttttt

SEQ ID NO: 47 - DNA, 54 bp, Artificial Sequence
  Synthetic Mouse Sering1 shRNA-10
gcagtgtagccatcacaatattcaagagatattgtgatggctacactgtttttt

SEQ ID NO: 48 - DNA, 19 bp, Artificial Sequence
  Synthetic Human SOD1 siRNA Hom si-5
ggcgtggcctagcgagtta

SEQ ID NO: 49 - DNA, 19 bp, Artificial Sequence
  Synthetic Human SOD1 siRNA Hom si-4
cattaaacactgtaatctt

SEQ ID NO: 50 - DNA, 19 bp, Artificial Sequence
  Synthetic Human SOD1 siRNA Hom si-11
gcatggattccatgttcat

SEQ ID NO: 51 - DNA, 19 bp, Artificial Sequence
  Synthetic Human SOD1 siRNA Hom si-12
gtgcagggcatcatcaatt

SEQ ID NO: 52 - DNA, 19 bp, Artificial Sequence
  Synthetic Human SOD1 siRNA Hom si-10
gcattaaaggactgactga

SEQ ID NO: 53 - DNA, 19 bp, Artificial Sequence
  Synthetic Human SOD1 siRNA Hom si-14
ccgatgtgtctattgaaga

SEQ ID NO: 54 - DNA, 19 bp, Artificial Sequence
  Synthetic Human SOD1 siRNA Hom si-13
ggccgatgtgtctattgaa

SEQ ID NO: 55 - DNA, 19 bp, Artificial Sequence
  Synthetic Human SOD1 siRNA Homo si-2
gcgtggcctagcgagttat

SEQ ID NO: 56 - DNA, 19 bp, Artificial Sequence
  Synthetic Human SOD1 siRNA Hom si-8
ggtggaaatgaagaaagta

SEQ ID NO: 57 - DNA, 19 bp, Artificial Sequence
  Synthetic Human SOD1 siRNA Hom si-6
cagtgcagggcatcatcaa

SEQ ID NO: 58 - DNA, 53 bp, Artificial Sequence
  Synthetic Human SOD1 shRNA-5
ggcgtggcctagcgagttattcaagagataactcgctaggccacgcctttttt

SEQ ID NO: 59 - DNA, 54 bp, Artificial Sequence
  Synthetic Human SOD1 shRNA-4
gcattaaacactgtaatcttagagaacttaagattacagtgtttaatgtttttt

SEQ ID NO: 60 - DNA, 53 bp, Artificial Sequence
  Synthetic Human SOD1 shRNA-11
gcatggattccatgttcatttcaagagaatgaacatggaatccatgctttttt

SEQ ID NO: 61 - DNA, 53 bp, Artificial Sequence
  Synthetic Human SOD1 shRNA-12
gtgcagggcatcatcaattagagaacttaattgatgatgccctgcactttttt

SEQ ID NO: 62 - DNA, 53 bp, Artificial Sequence
  Synthetic Human SOD1 shRNA-10
gcattaaaggactgactgattcaagagatcagtcagtcctttaatgctttttt

SEQ ID NO: 63 - DNA, 54 bp, Artificial Sequence
  Synthetic Human SOD1 shRNA-14
gccgatgtgtctattgaagattcaagagatcttcaatagacacatcggtttttt

SEQ ID NO: 64 - DNA, 53 bp, Artificial Sequence
  Synthetic Human SOD1 shRNA-13
ggccgatgtgtctattgaattcaagagattcaatagacacatcggcctttttt

SEQ ID NO: 65 - DNA, 53 bp, Artificial Sequence
  Synthetic Human SOD1 shRNA-2
gcgtggcctagcgagttatttcaagagaataactcgctaggccacgctttttt

SEQ ID NO: 66 - DNA, 53 bp, Artificial Sequence
  Synthetic Human SOD1 shRNA-8
ggtggaaatgaagaaagtattcaagagatactttcttcatttccacctttttt

SEQ ID NO: 67 - DNA, 54 bp, Artificial Sequence
  Synthetic Human SOD1 shRNA-6
gcagtgcagggcatcatcaattcaagagattgatgatgccctgcactgtttttt

SEQ ID NO: 68 - DNA, 465 bp, Artificial Sequence
  Synthetic Human SOD1 codon optimized cDNA SOD1-2
atggccaccaaggctgtgtgtgtgctgaagggggatggccctgtgcaaggcatcatcaactttgagcaaaaggagagcaatggccctgtgaaggtgtggggcagcatcaagggcctgacagagggcctgcatggcttccatgtgcatgagtttggggacaacacagctggctgcacttctgctggcccccacttcaaccccctgtctagaaagcatgggggccccaaggatgaggagagacatgtgggggacctgggcaatgtgacagctgacaaggatggggtggctgatgtgagcattgaggactctgtgatcagcctgtctggggaccactgcatcattggcagaaccctggtggtgcatgagaaggctgatgacctggggaaggggggcaatgaggagagcaccaagactggcaatgctggcagcagactggcctgtggggtgattggcattgctcagtga

SEQ ID NO: 69 - DNA, 465 bp, Artificial Sequence
  Synthetic Human SOD1 codon optimized cDNA SOD1-4
atggctaccaaggctgtgtgtgtgctgaagggggatggccctgtgcaaggcatcatcaactttgagcagaaggagagcaatggccctgtgaaggtgtggggcagcatcaagggcctgacagagggcctgcatggcttccatgtgcatgagtttggggacaacacagctggctgcacctctgctggcccccacttcaaccccctgagcagaaagcatgggggccccaaggatgaggagagacatgtgggggacctgggcaatgtgacagctgacaaggatggggtggctgatgtgagcatagaggactctgtgatcagcctgtctggggaccattgcatcattggcagaaccctggtggtgcatgagaaggcagatgacctgggcaaggggggcaatgaggagagcaccaagactggcaatgctggctctagactggcctgtggggtgattggcattgctcagtga

SEQ ID NO: 70 - DNA, 465 bp, Artificial Sequence
  Synthetic Human SOD1 codon optimized cDNA SOD1-9
atggccaccaaggctgtgtgtgtgctgaagggggatggccctgtgcaaggcatcatcaactttgagcagaaggagagcaatggccctgtgaaggtgtggggcagcatcaagggcctgacagagggcctgcatggcttccatgtgcatgagttcggggacaacacagctggctgcacatctgctggcccccacttcaaccccctgagcagaaagcatgggggccccaaggatgaggagagacatgtgggggacctgggcaacgtgacagctgacaaggatggggtggctgatgtgtctattgaggactctgtgataagcttgtctggggaccactgcatcattggcagaaccttggtggtgcatgagaaggctgatgacctgggcaaggggggcaatgaggagagcaccaagactggcaatgctggcagcagactggcttgtggggtgattggcattgctcagtga

SEQ ID NO: 71 - DNA, 465 bp, Artificial Sequence
  Synthetic Human SOD1 codon optimized cDNA SOD1-16
atggccaccaaggctgtgtgtgtgctgaagggggatggccctgttcaaggcatcatcaactttgagcagaaggaaagcaatggccctgtgaaggtgtggggctccatcaagggcctgacagagggcctgcatggctttcatgtgcatgagttcggggacaacactgctggctgtacatctgctggcccccacttcaaccccctgagcagaaagcatgggggccccaaggatgaggagagacatgtgggggacctgggcaatgtgacagctgacaaggatggggtggctgatgtgagcattgaggactctgtgatcagcctgtctggggaccactgcatcattggcagaaccctggtggtgcatgagaaggctgatgacctgggcaaggggggcaatgaggagagcaccaagactggcaatgctggcagcagactggcctgtggggtgattggcattgctcagtga

SEQ ID NO: 72 - DNA, 465 bp, Artificial Sequence
  Synthetic Human SOD1 codon optimized cDNA SOD1-18
atggccaccaaggctgtgtgtgtgctcaagggggatggccctgtgcaaggcatcatcaactttgagcaaaaggagagcaatggccctgtgaaggtgtggggcagcatcaagggactgacagagggcctgcatggcttccatgtgcatgagttcggggacaacacagctggctgtacatctgctggcccccacttcaatcccctgagcagaaagcatgggggccccaaagatgaggagagacatgtgggggacctgggcaatgtgacagctgacaaggatggggtggctgatgtgagcatcgaggactctgtgatcagcctctctggggaccactgcatcattggcagaaccctggtggtgcatgagaaggctgatgacctggggaaggggggcaatgaggagagcaccaagactggcaatgctggcagcagactggcctgtggggtgattggcatagctcagtga

SEQ ID NO: 73 - DNA, 465 bp, Artificial Sequence
  Synthetic Human SOD1 codon optimized cDNA SOD1-19
atggccaccaaggctgtgtgtgtgctgaagggggatggccctgtgcaaggcatcatcaactttgagcaaaaggagagcaatggccctgtgaaggtgtggggcagcatcaagggcctgactgagggcctgcatggcttccatgtgcatgagttcggggacaacacagctggctgcacatctgctggcccccacttcaaccccctgagcagaaagcatgggggccccaaggatgaggagagacatgtgggggacctgggcaatgtgacagctgacaaggacggggtggctgatgtgagcattgaggactctgtgatcagcctgtctggggaccactgcatcataggcagaaccctggtggtgcatgagaaggctgatgacctgggcaaggggggcaatgaggagtccaccaagactggcaatgctggcagcagactggcctgtggggtgattggcattgctcagtga

(19) United States
(12) Patent Application Publication
(10) Pub. No.: US 2022/0098615 A1
(43) Pub. Date: Mar. 31, 2022

(54) DUAL FUNCTIONAL EXPRESSION VECTORS AND METHODS OF USE THEREOF

(71) Applicant: NGGT, Inc., Sacramento, CA (US)

(72) Inventor: Lixin JIANG, Sacramento, CA (US)

(21) Appl. No.: 17/456,239

(22) Filed: Nov. 23, 2021

Related U.S. Application Data
(60) Provisional application No. 63/085,522, filed on Sep. 30, 2020.
(60) Provisional application No. 63/149,533, filed on Feb. 15, 2021.

(51) Int. Cl. C12N 15/86; C12N 15/113

(52) U.S. Cl. CPC C12N 15/86; C12N 2310/14; C12N 15/113

(57) ABSTRACT

A dual functional expression vector is disclosed. The dual functional vector comprises a nucleic acid suitably configured for expressing at least two active agents, where a first active agent is an siRNA/shRNA or miRNA for silencing endogenous mRNA transcripts from an endogenous gene, where a second active agent is a translation product of an allele of the endogenous gene being silenced, where the translation product is expressed from an mRNA transcript that is insensitive to the silencing activity of the first active agent. Also describes are methods of making the dual functional expression vector and methods of using the expression vector for treatment of diseases and for generating animal models of human diseases.

This application claims priority to U.S. Provisional Application Ser. No. 63/085,522, filed Sep. 30, 2020 and U.S. Provisional Application Ser. No. 63/149,533, filed Feb. 15, 2021, the contents of which are expressly incorporated by reference herein.

### FIELD

The present application generally relates to recombinant viruses for gene therapy. More particularly, the present application relates to a dual functional recombinant virus for treatment of single gene hereditary diseases caused by autosomal dominant negative, X-linked dominant, or X-linked recessive mutations.

### BACKGROUND

Considering there are thousands of genes in a mammalian genome, it is not surprising that there are many monogenetic diseases caused by one or more inherited and/or acquired mutations in one or both copies of a single gene. Although, the field of gene therapy has been made much progress in recent years, a few problems have been observed in the context of treating monogenetic diseases.

Recently, several approaches have been developed to target and selectively silence or knockdown expression of mutant gene alleles by RNA interference (RNAi). RNAi is a natural cellular process that utilizes siRNAs or miRNAs to silence gene expression by promoting the degradation of mRNA. It plays an important role in gene regulation and innate defense against invading viruses.

Despite the promise of RNAi, in many instances gene silencing approaches for treating genetic disease have been unsuccessful due to insufficiency of protein levels following silencing of the mutant alleles. To confer the knock-down efficacy for all the mutations of a particular gene, the silencer construct usually is designed to target the un-mutated common region of the sequence, which results in the degradation of all mRNA products of the gene, including the wild-type mRNA.

Other attempts to treat genetic diseases have sought to fix the target protein insufficiency by supplying exogenously transferred expression constructs to supplement (and increase) the expression levels of the wild type alleles so as to obviate the deficits attributed to expression (or lack thereof) of the mutant allele. However, this approach has encountered pitfalls due to the gain of function effects conferred by mutant alleles, such as toxicity and dominant negative activity against the wild type allele.

According, there is a need in the art for new methodologies addressing the foregoing problems to improve the efficacy of gene therapy for genetic diseases. The present application addresses the foregoing problems by providing a dual functional recombinant virus for treatment of monogenic diseases, especially those caused by autosomal dominant, X-linked dominant and X-linked recessive mutations, and for development of animal models for these genetic diseases.

Pre-clinical animal models are used as surrogates of human biology due to the logistical and ethical restrictions. To better understand human health and disease, researchers need to create models that carry human DNA and/or express human proteins. Such humanized animal models facilitate the dissection of disease mechanisms and enable evaluation of therapeutic candidates in an in vivo setting more relevant to human physiology.

There are many promising biomedical research applications for human therapeutics requiring humanized animal models, such as cancer, infectious diseases, immune and inflammation diseases, regenerative medicine, hematology, etc.

Transgenic mice were first generated to serve this purpose. These mice carry multicopy of human complementary DNAs (cDNAs) or mini-genes inserted at non-endogenous loci. However, concerns have arisen from these mice as overexpression artefacts, ectopic expression, random integration, variation in gene copy numbers, as well as the interfere from the endogenous mouse genes.

To avoid these problems, targeted genomic humanization of mice was developed in which a mouse sequence is deleted (knock-out) or replaced by the human orthologous DNA (knock-in). The initial development of knock-out and knock-in mice was relied primarily on embryonic stem cell (ESC) based gene targeting. In ESC based gene-editing technology, targeted mutations occur when homologous recombination (HR) occurs between a targeting vector and ESC genomic DNA (ESC/HR). Targeted ESCs are microinjected into blastocysts, which are then implanted into foster animals to produce founder mice. Besides time, cost and labor consuming, this technology is limited to mice because only mouse germline transmittable ESCs are available.

Therefore, there still exists a need of new methods to develop new animal models for biomedical research applications.

### SUMMARY

One aspect of the present application provides a dual functional expression vector. The dual functional vector comprises a nucleic acid suitably configured for expressing at least two active agents: a first active agent and a second active agent. The first active agent is a siRNA or shRNA or miRNA for silencing endogenous mRNA transcripts from an endogenous gene. The second active agent is the translation product of an allele (including a species homolog) of the endogenous gene being silenced, where the translation product is expressed from an mRNA transcript that is insensitive to the silencing activity of the first active agent.

In some embodiments, the present application provides a dual functional expression vector suitably configured for expressing at least two active agents, where a first active agent is an siRNA/shRNA (small hairpin RNA) or miRNA for silencing endogenous mRNA transcripts expressed from the mutant and wild-type alleles in a patient suffering from a genetic disease, where a second active agent is a coding sequence corresponding to a wild-type polypeptide of the gene being silenced, where the nucleic acid is genetically engineered to produce an mRNA transcript that is insensitive to the silencing activity of the first active agent.

In one embodiment, the genetic disease is caused by one or more autosomal dominant mutations.

In another embodiment, the genetic disease is caused by one or more X-linked dominant mutations.

In another embodiment, the genetic disease is caused by one or more X-linked recessive mutations.

In another embodiment, the genetic disease is listed in Table 1 and the first and second active agents are directed to the corresponding gene or polypeptide associated therewith.

In another embodiment, the genetic disease is listed in Table 2 and the first and second active agents are directed to the corresponding gene or polypeptide associated therewith.

In a particular embodiment, the genetic disease is familial amyotrophic lateral sclerosis (fALS), where the siRNA/shRNA or miRNA is targeted for silencing endogenous (mutant and wild-type) superoxide dismutase 1 (SOD1) mRNA transcripts, and where the second active agent is a siRNA/shRNA/miRNA-resistant wild-type SOD1 coding sequence.

In another embodiment, the genetic disease is familial amyotrophic lateral sclerosis (fALS), where the siRNA/shRNA or miRNA is targeted for silencing endogenous (mutant and wild-type) C9orf72 transcripts, and where the second active agent is a siRNA/shRNA/miRNA-resistant wild-type C9orf72 coding sequence.

In another embodiment, the genetic disease is hereditary angioedema (HAE), wherein the siRNA/shRNA or miRNA is targeted for silencing endogenous (mutant and wild-type) C1 esterase inhibitor transcripts, and wherein the second active agent is a siRNA/shRNA/miRNA-resistant wild-type C1 esterase inhibitor coding sequence.

In some embodiments, the expression vector is a recombination virus.

In some embodiments, the recombinant virus is an adenovirus-associated virus (AAV), a retrovirus, a lentivirus, a herpes virus or an adenovirus.

In a particular embodiment, the recombinant virus is a recombinant AAV virus (rAAV).

In another aspect, the present application provides a pharmaceutical composition comprising one of the foregoing recombinant viruses in combination with a pharmaceutically acceptable carrier.

In another aspect, the present application provides a method for treating a genetic disease, comprising: administering an effective amount of a recombinant virus in accordance with the present application into a subject in need thereof, where the subject has a genetic disease listed in Table 1, Table 2 or Table 3 as shown below.

TABLE 1
Autosomal dominant diseases and corresponding genes/proteins

| Disease | Gene/Defect | Protein/Defect | Clinical Features etc |
|---|---|---|---|
| Achondroplasia | FGFR3 | Fibroblast growth factor | Short limbs relative to |
| | | receptor 3 | trunk, prominent |
| | | | forehead, low nasal root, |
| | | | redundant skin folds on |
| | | | arms and legs; |
| | | | constitutively active |
| | | | (gain of function) |
| Autosomal | ATXN1, ATXN2, | Multiple proteins | Group of progressive |
| dominant cerebellar | ATXN3, SPTBN2, | corresponding to each of | neurological genetic |
| ataxias (ADCAs) | CACNA1A, ATXN7, | the aforementioned | disorders characterized |
| | ATXN8, ATXN10, | genes | by poor movement |
| | TTBK12, PPP2R2B, | | coordination (ataxia) of |
| | KCNC3, PRKCG, | | hands, speech, and eye |
| | ITPR1, KCND3, | | movements, especially a |
| | TMEM240, PDYN, | | jerky, unsteady walking |
| | SCA25, EEF2, FGF14, | | style (gait). Frequently, |
| | AFG3L2, BEAN1, | | atrophy of the cerebellum |
| | ELOVL4, TGM6, | | occurs. |
| | NOP56, ELOVL5, | | |
| | CCDC88C, TRPC3, | | |
| | CACNA1G, MME, | | |
| | GRM1, FAT2, PLD3, | | |
| | PUM1, STUB1, ATN1, | | |
| | DNMT1, TUBB4A, | | |
| | GRID2, C9orf72, | | |
| | FGF12, ATP1A3 | | |
| Autosomal dominant | DNM2 | Dynamin-2 | |
| centronuclear myopathy-1 | | | |
| Autosomal dominant | DNM2 | Dynamin-2 | |
| intermediate Charcot- | | | |
| Marie-Tooth disease type | | | |
| B (CMTDIB) | | | |
| Autosomal dominant | YARS1 | Tyrosyl-tRNA | |
| intermediate Charcot- | | synthetase | |
| Marie-Tooth disease type | | | |
| C (CMTDIC) | | | |
| Autosomal dominant | MPZ | Myelin protein P0 | |
| intermediate Charcot- | | | |
| Marie-Tooth disease type | | | |
| D (CMTDID) | | | |
| Autosomal dominant | INF2 | Inverted formin-2 | |
| intermediate Charcot- | | | |
| Marie-Tooth disease type | | | |
| E (CMTDIE) | | | |
| Autosomal dominant | GNB4 | Guanine nucleotide- | |
| intermediate Charcot- | | binding protein subunit | |
| Marie-Tooth disease type | | beta-4 | |
| E (CMTDIF) | | | |
| Autosomal dominant | NEFL | Neurofilament light | |
| intermediate Charcot- | | polypeptide | |
| Marie-Tooth disease type | | | |
| E (CMTDIG) | | | |
| Autosomal dominant | SYNGAP1 | Ras/Rap GTPase- | |
| mental retardation-5 | | activating protein | |
| (MRD5) | | SynGAP | |
| Autosomal dominant | PKD1 | Polycystin-1 | Most common inherited |
| polycystic kidney disease | PKD2 | Polycystin-2 | disorder of kidneys; |
| (ADPKD) | GANAB (less | | characterized by growth |
| | frequently) DNAJB11 | | of numerous cysts in the |
| | (less frequently) | | kidneys; symptoms vary |
| | | | in severity and age of |
| | | | onset, but usually |
| | | | develop between the ages |
| | | | of 30 and 40; ADPKD is |
| | | | a progressive disease |
| | | | with symptoms getting |
| | | | worse over time, often |
| | | | leading to renal failure |
| | | | requiring dialysis |
| Autosomal dominant | VAMP1 | Vesicle-associated | |
| spastic ataxia-1 (SPAX1) | | membrane protein 1 | |
| Cancers (various) | TP53 | Cellular tumor antigen | Frequently mutated or |
| | | p53 | inactivated in about 60% |
| | | | of cancers; autosomal |
| | | | dominant (e.g., dominant |
| | | | negative) and autosomal |
| | | | recessive mutations; four |
| | | | types of cancers account |
| | | | for 80% of tumors |
| | | | occurring in TP53 |
| | | | germline mutation |
| | | | carriers: breast cancers, |
| | | | soft tissue and bone |
| | | | sarcomas, brain tumors |
| | | | (astrocytomas) and |
| | | | adrenocortical |
| | | | carcinomas; less frequent |
| | | | tumors include choroid |
| | | | plexus carcinoma or |
| | | | papilloma before the age |
| | | | of 15, |
| | | | rhabdomyosarcoma |
| | | | before the age of 5, |
| | | | leukemia, Wilms tumor, |
| | | | malignant phyllodes |
| | | | tumor, colorectal and |
| | | | gastric cancers. |
| Charcot-Marie-Tooth | GJB1 | Connexin 32 (Cx32) | Characterized by a |
| disease 1 (CMTX1) | | | slowly progressive |
| | | | course: muscle wasting |
| | | | and weakness of distal |
| | | | limb muscles mainly |
| | | | involving the feet, legs |
| | | | and hands (particularly |
| | | | the thenar eminence), |
| | | | with proximal muscle |
| | | | weakness occurring in |
| | | | severe cases; distal |
| | | | sensory loss; loss of deep |
| | | | tendon reflexes |
| Charcot-Marie-Tooth | PMP22 | Peripheral myelin | Typical features include |
| disease type 1E | | protein 22 | progressive weakness of |
| (CMT1E) | | | the feet and/or |
| | | | ankles; foot drop; |
| | | | atrophy of muscles below |
| | | | the knee; absent tendon |
| | | | reflexes of upper and |
| | | | lower extremities; and a |
| | | | decreased sensitivity to |
| | | | touch, heat, and cold in |
| | | | the feet and/or lower |
| | | | legs) in addition |
| | | | to hearing loss |
| Cone-rod dystrophy | AIPL1 | | |
| Cone-rod dystrophy-2 | CRX | | |
| (CORD2) | | | |
| Cone-rod dystrophy-6 | GUCY2D | | |
| (CORD6) | | | |
| Early onset familial | APP | Amyloid precursor | Degenerative disease of |
| autosomal dominant | | protein | brain causing gradual |
| Alzheimer disease Type 1 | | | loss of memory, |
| | | | judgement, and the |
| | | | ability to function |
| | | | socially |
| Early onset familial | PSEN1 | Presenilin 1 | Degenerative disease of |
| autosomal dominant | | | brain causing gradual |
| Alzheimer disease Type 3 | | | loss of memory, |
| | | | judgement, and the |
| | | | ability to function |
| | | | socially |
| Early onset familial | PSEN2 | Presenilin 2 | Degenerative disease of |
| autosomal dominant | | | brain causing gradual |
| Alzheimer disease Type 4 | | | loss of memory, |
| | | | judgement, and the |
| | | | ability to function |
| | | | socially |
| Emery-Dreifuss muscular | EMD | Emerin | Musculoskeletal |
| dystrophy (EDMD) | | | disorder; muscular |
| | | | dystrophy |
| Emery-Dreifuss muscular | LMNA | Lamins A/C | Musculoskeletal |
| dystrophy | | | disorder; muscular |
| | | | dystrophy |
| Familial amylotrophic | SOD1 | Superoxide dismutase 1 | Fatal progressive |
| lateral sclerosis (fALS) | | enzyme | neurodegenerative |
| | | | disease, characterized by |
| | | | loss of motor neurons |
| | | | (MNs) in primary motor |
| | | | cortex, brainstem, and |
| | | | spinal cord; onset of |
| | | | disease typically occurs |
| | | | between ages of 40-70; |
| | | | early onset form/disease |
| | | | has been found in |
| | | | patients between the ages |
| | | | of 20-30 |
| Familial amylotrophic | C9orf72 | Guanine nucleotide | Fatal progressive |
| lateral sclerosis (fALS) | | exchange C9orf72 | neurodegenerative |
| | | | disease, characterized by |
| | | | loss of motor neurons |
| | | | (MNs) in primary motor |
| | | | cortex, brainstem, and |
| | | | spinal cord; onset of |
| | | | disease typically occurs |
| | | | between ages of 40-70; |
| | | | early onset form/disease |
| | | | has been found in |
| | | | patients between the ages |
| | | | of 20-30 |
| Familial atrial fibrillation | KCNQ1 | | Atrial fibrillation (AF) is |
| 3 (ATFB3) | KCNE2 | | the most common |
| Familial atrial fibrillation | NPPA | | sustained cardiac rhythm |
| 4 (ATFB4) | KCNA5 | | disturbance, affecting |
| Familial atrial fibrillation | KCNJ2 | | more than 2 million |
| 6 (ATFB6) | SCN5A | | Americans, with an |
| Familial atrial fibrillation | GJA5 | | overall prevalence of |
| 7 (ATFB7) | ABCC9 | | 0.89%. The prevalence |
| Familial atrial fibrillation | SCN1B | | increases rapidly with |
| 9 (ATFB9) | SCN2B | | age, to 2.3% between the |
| Familial atrial fibrillation | NUPP155 | | ages of 40 and 60 years, |
| 10 (ATFB10) | SCN3B | | and to 5.9% over the age |
| Familial atrial fibrillation | SCN4B | | of 65. The most dreaded |
| 11 (ATFB11) | MYL4 | | complication is |
| Familial atrial fibrillation | | | thromboembolic stroke |
| 12 (ATFB12) | | | |
| Familial atrial fibrillation | | | |
| 13 (ATFB13) | | | |
| Familial atrial fibrillation | | | |
| 14 (ATFB14) | | | |
| Familial atrial fibrillation | | | |
| 15 (ATFB15) | | | |
| Familial atrial fibrillation | | | |
| 16 (ATFB16) | | | |
| Familial atrial fibrillation | | | |
| 17 (ATFB17) | | | |
| Familial atrial fibrillation | | | |
| 18 (ATFB18) | | | |
| Hereditary angioedema | SERPING1 | C1 esterase inhibitor | Disease characterized by |
| (HAE) | | (C1-INH) | recurrent episodes (or |
| | | | attacks) of severe |
| | | | swelling of the skin and |
| | | | mucous membranes (e.g., |
| | | | gastrointestinal tract and |
| | | | upper airway). Most |
| | | | people have their first |
| | | | attack in childhood or |
| | | | adolescence. |
| Familial exudative | FZD4 or | Frizzled class receptor 4; | Progressive vision loss |
| vitreoretinopathy (FEVR) | LRP5 | LRP5 protein | |
| Familial | LDLR1 | Low-density lipoprotein | Impaired uptake of LDL, |
| hypercholesterolemia 1 | | receptor | elevated levels of LDL |
| | | | cholesterol, |
| | | | cardiovascular disease |
| | | | and stroke. Symptoms |
| | | | more severe in |
| | | | homozygous individuals |
| Familial | APOB | Apolipoprotein B-100 | Hypercholesterolemia |
| hypercholesterolemia 2 | | | and increased proneness |
| | | | to coronary artery disease |
| | | | (CAD). Plasma |
| | | | cholesterol levels are |
| | | | dramatically elevated due |
| | | | to impaired clearance of |
| | | | LDL particles by |
| | | | defective APOB/E |
| | | | receptors. |
| Familial | PCSK9 | Proprotein convertase | Disorder of lipid |
| hypercholesterolemia 3 | | subtilisin/kexin type 9 | metabolism characterized |
| | | | by a selective increase of |
| | | | low density lipoprotein |
| | | | particles in plasma, |
| | | | giving rise to tendon and |
| | | | skin xanthomas, arcus |
| | | | comeae, and coronary |
| | | | artery disease |
| GNE-related myopathy | GNE | UDP-GlcNAc-2- | |
| | | epimerase/ManAc | |
| | | kinase | |
| Hepatocyte nuclear factor | HNF1B | Hepatocyte nuclear | Kidney abormalities |
| 1 beta-associated disease | | factor-1 beta (HNF-1β) | |
| (HNF1B-associated | | | |
| diseases) | | | |
| Hereditary hemorrhagic | ACVRL1 | ALK-1 (or | Blood disorder that can |
| telangiectasia (HHT) | ENG | SKR3)EndoglinMAD | cause excessive bleeding |
| Syndrome) | SMAD4 | homolog 4GDF-2 | and development of |
| | GDF2 | | abnormal blood vessels |
| | | | (arteriovenous |
| | | | malformations (AVMs)) |
| | | | in several areas of the |
| | | | body, including skin |
| | | | (called telangiectasias), |
| | | | brain, lungs, liver, and |
| | | | intestines. |
| Hereditary pancreatitis | PRSS1 | Serine protease 1 | Causes multiple episodes |
| | | | of inflammation of the |
| | | | pancreas (pancreatitis); |
| | | | signs and symptoms |
| | | | include stomach pain, |
| | | | nausea, vomiting, fatty |
| | | | stools, weight loss, and |
| | | | poor absorption of |
| | | | nutrients from food; |
| | | | adults have increaseed |
| | | | risk type 1 diabetes and |
| | | | pancreatic cancer |
| Hereditary sensory and | SCN11A | Sodium channel protein | Characterized by |
| autonomic neuropathy | | type 11 subunit alpha | congenital inability to |
| type 7 (HSAN7) | | | experience pain resulting |
| | | | in self-mutilations, slow- |
| | | | healing wounds, and |
| | | | multiple painless |
| | | | fractures, mild muscle |
| | | | weakness, delayed motor |
| | | | development, slightly |
| | | | reduced motor and |
| | | | sensosy nerve conduction |
| | | | velocities, hyperhidrosis |
| | | | and gastrointestinal |
| | | | dysfunction |
| Hereditary sensory | SPTLC1 | Serine | Neurological condition |
| neuropathy type 1 | | palmitoyltransferase | characterized by nerve |
| (HSN1) | | (SPT) | abnormalities in the legs |
| | | | and feet, including |
| | | | tingling, weakness, and a |
| | | | reduced ability to feel |
| | | | pain and sense hot and |
| | | | cold |
| Hereditary Spherocytosis | ANK1 (half of cases); | Ankyrin-1 | Condition characterized |
| | other genes include | | by hemolytic anemia; |
| | EPB42, SLC4A1, | | signs/symptoms range |
| | SPTA1, and SPTB | | from mild to severe and |
| | | | may include pale skin, |
| | | | fatigue, anemia, jaundice, |
| | | | gallstones, and/or |
| | | | enlargement of the spleen |
| Holoproencephaly | SHH | Sonic hedgehog | Malformation of the |
| | | | brain (no or reduced |
| | | | evidence of an |
| | | | interhemispheric fissure), |
| | | | dysmorphic facial |
| | | | features, mental |
| | | | retardation |
| Huntington Disease | HTT | Huntingtin | Disorder is characterized |
| (Also Huntington | | | by progressive motor, |
| Chorea) | | | cognitive and psychiatric |
| | | | abnormalities. Chorea - |
| | | | nonrepetitive involuntary |
| | | | jerks - is observed in |
| | | | 90% of patients; CAG |
| | | | repeat expansion within |
| | | | exon 1 |
| Hypophosphatemic | PHEX | PHEX enzyme | Endocrine/developmental |
| rickets (or X-linked | | | disorder |
| hypophosphatemia, | | | |
| XLH) | | | |
| Juvenile retinitis | AIPL1 | | |
| pigmentosa | | | |
| Late-onset retinal | C1QTNF5 | | |
| degeneration (LORD) | | | |
| Machado-Joseph disease | ATXN3 | ATXN3 enzyme | Inherited form of ataxia; |
| (Spinocerebellar ataxia 3 | | | signs/symptoms may |
| (SCA3)) | | | begin between childhood |
| | | | and late adulthood and |
| | | | vary greatly and may |
| | | | include slowly |
| | | | progressive clumsiness in |
| | | | the arms and legs; a |
| | | | manner of walking (gait) |
| | | | that may be mistaken for |
| | | | drunkenness; difficulty |
| | | | speaking and |
| | | | swallowing; impaired |
| | | | eye movements or vision; |
| | | | lower limb spasticity; |
| | | | dystonia or symptoms |
| | | | similar to those of |
| | | | Parkinson's |
| | | | disease; twitching of the |
| | | | face or tongue; nerve |
| | | | damage (neuropathy); |
| | | | and/or problems with |
| | | | urination and the |
| | | | autonomic nervous |
| | | | system |
| Marfan's Syndrome | FBN1 | Fibrillin-1 | Abnormalities of the |
| | | | skeleton |
| | | | (disproportionate tall |
| | | | stature, scoliosis), heart |
| | | | (mitral valve prolapse, |
| | | | aortic dilatation, |
| | | | dissection of the |
| | | | ascending aorta), |
| | | | pulmonary system, skin |
| | | | (excessive elasticity), and |
| | | | joints (hypermobility). A |
| | | | frequent cause of death is |
| | | | congestive heart failure. |
| Medullary cystic kidney | MUC1 | Mucin-1 | Progressive loss of |
| disease type 1 (MCKD1) | | | kidney function; slowly |
| | | | progressive kidney |
| | | | disease that usually |
| | | | becomes apparent during |
| | | | childhood |
| Medullary thyroid | RET | Ret tyrosine-protein | Three types: (a) multiple |
| carcinoma (MTC) | | kinase receptor | neoplasia type 2A |
| | | | (MEN2A), (b) multiple |
| | | | neoplasia type IIB |
| | | | (MEN2B) and (c) |
| | | | familial MTC (FMTC), |
| | | | which occurs in 25-30% |
| | | | of MTC cases and where |
| | | | MTC is the only clinical |
| | | | manifestation |
| Myotonic Dystrophy | DMPK | Myotonin protein kinase | Disorder shows |
| | | | anticipation. Muscle |
| | | | weakness, cardiac |
| | | | arrhythmias, cataracts |
| | | | and testicular atrophy in |
| | | | males; children born with |
| | | | congenital form have a |
| | | | characteristic open |
| | | | triangle-shaped mouth; |
| | | | CTG repeat expansion in |
| | | | 3′ untranslated region of |
| | | | the gene |
| Neurofibromatosis I | NF1 | Neurofibromin | The disorder is |
| | | | characterized by |
| | | | numerous benign tumors |
| | | | (neurofibromas) of the |
| | | | peripheral nervous |
| | | | system, but a minority of |
| | | | patients also show |
| | | | increased incidence of |
| | | | malignancy |
| | | | (neurofibrosarcoma, |
| | | | astrocytoma, Schwann |
| | | | cell cancers and |
| | | | childhood CML - |
| | | | chronic myelogenous |
| | | | leukemia) |
| Osteogenesis Imperfecta | Either of the genes | | Null mutations produce a |
| | encoding the α1 or α2 | | milder form of the |
| | chains of type I collagen | | disease. Missense |
| | | | mutations that act in a |
| | | | dominant negative |
| | | | manner are often |
| | | | perinatal lethal. The |
| | | | disorders are associated |
| | | | with deformed, |
| | | | undermineralized bones |
| | | | that are subject to |
| | | | frequent fracture. |
| Parkinson's disease | SNCA (PARK1) | Alpha-synuclein | Progressive neurologic |
| | | | disease affecting |
| | | | movement. Four main |
| | | | symptoms: tremors of the |
| | | | hands, arms, legs, jaw, or |
| | | | head, specially at |
| | | | rest; rigidity, or |
| | | | stiffness; bradykinesia, or |
| | | | slow movement; |
| | | | and postural instability or |
| | | | inability to find balance |
| Parkinson's disease | LRRK2 (PARK8) | Leucine-rich repeat | Progressive neurologic |
| | | serine/threonine-protein | disease affecting |
| | | kinase 2 (Dardarin) | movement. Four main |
| | | | symptoms: tremors of the |
| | | | hands, arms, legs, jaw, or |
| | | | head, specially at |
| | | | rest; rigidity, or |
| | | | stiffness; bradykinesia, or |
| | | | slow movement; |
| | | | and postural instability or |
| | | | inability to find balance |
| Parkinson's disease | VPS35 (PARK17) | Vacuolar protein | Progressive neurologic |
| | | sorting-associated | disease affecting |
| | | protein 35 | movement. Four main |
| | | | symptoms: tremors of the |
| | | | hands, arms, legs, jaw, or |
| | | | head, specially at |
| | | | rest; rigidity, or |
| | | | stiffness; bradykinesia, or |
| | | | slow movement; |
| | | | and postural instability or |
| | | | inability to find balance |
| REN-related kidney | REN | Renin protein | Progressive loss of |
| disease | | | kidney function; kidneys |
| | | | become less able to filter |
| | | | fluids and waste products |
| | | | from the body, resulting |
| | | | in kidney failure |
| Retinal vasculopathy with | TREX1 | Three-prime repair | Central nervous system |
| cerebral leukodystrophy | | exonuclease 1 | disorder; symptoms |
| (RVCL) | | | begin in adulthood |
| | | | (usually in the 40s) and |
| | | | may include loss of |
| | | | vision, mini-strokes, and |
| | | | dementia |
| Retinitis pigmentosa 1 | RP1 | Oxygen-regulated | |
| (RP1) | | protein 1 (or RP1 | |
| | | protein) | |
| Retinitis pigmentosa 4 | RHO | | |
| (RP4) | | | |
| Retinitis pigmentosa 7 | PRPH2 | | |
| (RP7) | | | |
| Retinitis pigmentosa 9 | RP9 | | |
| (RP9) | | | |
| Retinitis pigmentosa 10 | IMPDH1 | | |
| (RP10) | | | |
| Retinitis pigmentosa 11 | PRPF31 | | |
| (RP11) | | | |
| Retinitis pigmentosa 13 | PRPF8 | | |
| (RP13) | | | |
| Retinitis pigmentosa 17 | CA4 | | |
| (RP17) | | | |
| Retinitis pigmentosa 18 | PRPF3 | | |
| (RP18) | | | |
| Retinitis pigmentosa 19 | ABCA4 | | |
| (RP19) | | | |
| Retinitis pigmentosa 27 | NRL | | |
| (RP27) | | | |
| Retinitis pigmentosa 30 | FSCN2 | | |
| (RP30) | | | |
| Retinitis pigmentosa 31 | TOPORS | | |
| (RP31) | | | |
| Retinitis pigmentosa 33 | SNRNP200 | | |
| (RP33) | | | |
| Retinitis pigmentosa 35 | SEMA4A | | |
| (RP35) | | | |
| Retinitis pigmentosa 37 | NR2E3 | | |
| (RP37) | | | |
| Retinitis pigmentosa 42 | KLHL7 | | |
| (RP42) | | | |
| Retinitis pigmentosa 44 | RGR | | |
| (RP44) | | | |
| Retinitis pigmentosa 48 | GUCA1B | | |
| (RP48) | | | |
| Retinitis pigmentosa 50 | RP50 | | |
| (RP50) | | | |
| Retinitis pigmentosa 53 | RDH12 | | |
| (RP53) | | | |
| Retinitis pigmentosa 60 | PRPF6 | | |
| (RP60) | | | |
| Retinitis pigmentosa 70 | PRPF4 | | |
| (RP70) | | | |
| Retinitis pigmentosa 83 | ARL3 | | |
| (RP83) | | | |
| Retinitis pigmentosa 87 | RPE65 | | |
| (RP87) | | | |
| Retinitis pigmentosa 89 | KIF3B | | |
| (RP89) | | | |
| Spinocerebellar ataxia 3 | ATXN3 | Ataxin-3 (ATX3) | Characterized by |
| (SCA3) | | | cerebellar ataxia in |
| | | | combination with |
| | | | additional clinical |
| | | | features like optic |
| | | | atrophy, |
| | | | ophthalmoplegia, bulbar |
| | | | and extrapyramidal signs, |
| | | | peripheral neuropathy |
| | | | and dementia; CAG |
| | | | repeat expansion in |
| | | | ATX3 coding region |
| Timothy syndrome (TS) | CACNA1C | Calcium voltage-gated | Timothy syndrome (TS) |
| | | channel subunit alpha1 | is characterized by |
| | | C | multiorgan dysfunction, |
| | | | including lethal |
| | | | arrhythmias, webbing of |
| | | | fingers and toes, |
| | | | congenital heart disease, |
| | | | immune deficiency, |
| | | | intermittent |
| | | | hypoglycemia, cognitive |
| | | | abnormalities, and autism |
| Transthyretin amyloidosis | TTR | Transthyretin | Slowly progressive |
| | | | condition characterized |
| | | | by buildup of abnormal |
| | | | deposits of amyloid |
| | | | protein (amyloidosis) in |
| | | | the body's organs and |
| | | | tissues, mostly in the |
| | | | peripheral nervous |
| | | | system, resulting in a |
| | | | loss of sensation in the |
| | | | extremities (peripheral |
| | | | neuropathy) |
| Tuberous Sclerosis 2 | TSC2 | Tuberin | Tubers (glial nodules), |
| | | | seizures, mental |
| | | | retardation. Associated |
| | | | with adenoma sebaceum |
| | | | (facial lesion), |
| | | | myocardial |
| | | | rhabdomyomas, renal |
| | | | angiomyolipomas |
| Uromodulin-associated | UMOD | Uromodulin | Progressive loss of |
| kidney disease | | | kidney function; |
| | | | abnormal buildup of |
| | | | uromodulin leads to |
| | | | apoptosis of kidney cells, |
| | | | causing kidney disease |
| Von Hippel-Lindau | VHL | von Hippel-Lindau | (1) Hemangioblastomas |
| Syndrome | | disease tumor suppressor | of cerebellum, medulla, |
| | | | or retina, (2) adenomas, |
| | | | (3) cysts in visceral |
| | | | organs. High risk for |
| | | | renal cell carcinoma. |
| Vascular Ehlers-Danlos | COL3A1 | Collagen type III alpha 1 | Connective tissue |
| Syndrome Type IV | | chain | disorder caused by |
| | | | defects in collagen; |
| | | | generally considered the |
| | | | most severe form of |
| | | | Ehlers-Danlos |
| | | | syndrome (EDS). |
| | | | Common symptoms |
| | | | include thin, translucent |
| | | | skin; easy bruising; |
| | | | characteristic facial |
| | | | appearance; and fragile |
| | | | arteries, muscles and |
| | | | internal organs |
| X-linked scapuloperoneal | FHL1 | Four and a half LIM | Muscular dystrophy |
| myopathy | | domains protein 1 | characterized by |
| | | | weakness and wasting of |
| | | | the muscles in the lower |
| | | | legs and the area of the |
| | | | shoulder blades |

TABLE 2
X-linked recessive diseases and corresponding genes/proteins

| Disease | Gene/Defect | Protein/Defect | Clinical Features etc |
|---|---|---|---|
| Adrenomyeloneuropathy | ABCD1 | Adrenoleukodystrophy | Metabolic disorder |
| (AMN) | | protein (ALDP) | |
| Alport syndrome | COL4A5 | Alpha5(IV) chain of | Genetic condition characterized |
| | | type IV collagen | by kidney disease, hearing loss, and |
| | | | eye abnormalities |
| Arts syndrome | PRPS1 | Phosphoribosyl | Sensorineural hearing loss and serious |
| | | pyrophosphate (PRPP) | neurological and immune |
| | | synthetase 1 | system problems |
| Barth syndrome | TAZ | Tafazzin | Metabolic/neuromuscular disorder; |
| | | | primarily affects the heart, immune |
| | | | system, muscles, and growth |
| Becker's muscular | DMD | Dystrophin | Progressive weakness and wasting of |
| dystrophy | | | the skeletal and cardiac (heart) |
| | | | muscles |
| Borjeson-Forssman- | PHF6 | PHD finger protein 6 | Genetic condition characterized |
| Lehmann syndrome (BF | | | by intellectual disability, obesity, |
| LS) | | | seizures, hypogonadism, developmental |
| | | | delay and distinctive facial features |
| Bruton's disease (X- | BTK | Bruton tyrosine kinase | Immune system disorder; primary |
| Linked | | | immunodeficiency |
| Agammaglobulinemia) | | | |
| Cabeza's syndrome | CUL4B | Cullin-4B | Nervous system disorder (from birth); |
| | | | mental retardation |
| Chondrodysplasia | ARSE (or | arylsulfatase E | Bone and cartilage development |
| punctata 1, X-linked | ARSL) | | |
| recessive (CDPX1) | | | |
| Choroideremia | CHM | Rab escort protein 1 | Vision loss; retinopathy; 1 in 50,000 |
| | | (REP1) | males |
| Chronic Granulomatous | CYBB | Cytochrome b-245, | Immune deficiency; Phagocyte |
| Disease | | beta chain (also known | Deficiency; |
| | | as p91-phox) | |
| Dent disease type 1 | CLCN5 | Chloride transporter | Chronic kidney disease |
| | | ClC-5 or chloride | |
| | | channel 5 (CCL5) | |
| | | protein | |
| Dent disease type 2 | OCRL | OCRL enzyme | Chronic kidney disease |
| DMD-associated dilated | DMD | Dystrophin | Low or no expression of dystrophin in |
| cardiomyopathy (DCM) | | | heart; primarily affects (weakening) |
| | | | heart muscles only, preventing the |
| | | | heart from pumping blood efficiently |
| Duchenne muscular | DMD | Dystrophin | Muscular dystrophy; progressive |
| dystrophy (DMD) | | | muscle wasting |
| Fabry disease | GLA | alpha-galactosidase A | Lysosomal storage disese; |
| | | enzyme | angiokeratomas (skin lesions) over |
| | | | lower trunk, fever, severe burning |
| | | | pain in extremities, cardiovascular and |
| | | | cerebrovascular involvement |
| Fragile X tremor/ataxia | FMR1 | Synaptic functional | Progressively expanded trinucleotide |
| syndrome (FXTAS) | | regulator FMR1 | repeats on the long arm of the X- |
| | | | chromosome |
| Fragile | AFF2 | AF4/FMR2 family | CCG trinucleotide expansion |
| XE syndrome (FRAXE) | | member 2 | |
| Glucose-6-Phosphate | G6PD | Glucose-6-Phosphate | RBC defect; Susceptibility to |
| Dehydrogenase (G6PD) | | Dehydrogenase | oxidative damage to RBC's, leading to |
| Deficiency | | (G6PD) | hemolytic anemia. Can be elicited by |
| | | | drugs (primaquine, sulfonamides, |
| | | | aspirin), fava beans (favism). More |
| | | | prevalent in blacks. |
| Hemophilia A (Factor | F8 | Coagulation Factor | Bleeding disorder |
| VIII Deficiency) | | VIII | |
| Hemophilia B (Factor | F9 | Coagulation Factor IX | Bleeding disorder |
| IX Deficiency) | | | |
| Hunter syndrome (or | IDS | Iduronate 2-sulfatase | Progressively debilitating disorder; |
| Mucopolysaccharidosis | | (I2S) enzyme | similar to but less severe than Hurler |
| type II (MPS II)) | | | Syndrome; hepatosplenomegaly, |
| | | | micrognathia, retinal degeneration, |
| | | | joint stiffness, mild retardation, |
| | | | cardiac lesions |
| Intellectual disability- | ZC4H2 | Zinc finger C4H2 | Nervous system disorder (from birth) |
| developmental delay- | | domain-containing | |
| contractures syndrome | | protein | |
| Isolated congenital | CHRDL1 | Bone morphogenetic | Eye disease |
| megalocomea | | protein 4 (BMP4) | |
| Juberg Marsidi | ATRX | Transcriptional | Nervous system disorder (from birth); |
| syndrome | | regulator ATRX | mental retardation |
| Juvenile retinoschisis | RS1 | Retinoschisin | Eye disease |
| Kallmann syndrome 1 | ANOS1 (of | Anosmin-1 | Endocrine disease; form |
| | KAL1) | | of hypogonadotropic hypogonadism; |
| | | | characterized by delayed or absent |
| | | | puberty, impaired sense of smell, |
| | | | color blindness, cleft lip or palate, |
| | | | abnormal eye movements, hearing |
| | | | loss, failure of one of the kidneys to |
| | | | develop, mirror image hand |
| | | | movements, abnormalities of tooth |
| | | | development, and infertility. |
| Menkes kinky hair | ATP7A | ATP7A protein | Progressive nervous system decline |
| disease | | | |
| MECP2 duplication | MECP2 | Methyl-CpG binding | Nervous system disorder; severe |
| syndrome | | protein 2 | neurological and developmental |
| | | | disorder caused by |
| | | | MECP2 duplication |
| Lesch-Nyhan Syndrome | HPRT1 | Hypoxanthine | Blood/kideny/urinary diseases; |
| | | Phosphoribosyltransferase | nitrogen metabolism defect; |
| | | (HPRT) | hyperuricemia (gout), mental |
| | | | retardation, self-mutilation (autistic |
| | | | behavior), choreoathetosis, spasticity |
| Mohr-Tranebjaerg | TIMM8A (or | TIMM8A (or DDP1) | Characterized by clinical |
| syndrome (MTS) | DDP1) | protein | manifestations commencing with |
| | | | early childhood onset hearing loss, |
| | | | followed by adolescent onset |
| | | | progressive dystonia or ataxia, visual |
| | | | impairment from early adulthood |
| | | | onwards and dementia from the 4th |
| | | | decade onwards |
| Monoamine oxidase A | MAOA | Monoamine oxidase A | Nervous system disorder |
| deficiency | | enzyme | characterized by mild intellectual |
| | | | disability and behavioral difficulties |
| | | | (including aggressive and sometimes |
| | | | violent behaviors and autistic |
| | | | features) |
| Nephrogenic diabetes | AVPR2 | Vasopressin V2 | Kidney/urinary disease |
| insipidus | | receptor | |
| Oligophrenin-1 | OPNH1 | Oligophrenin-1 | Neurological |
| (OPHN1) syndrome | | | disorder/development/mental |
| | | | retardation |
| Ornithine | OTC | Ornithine | Nervous system disease; causes |
| transcarbamylase (OTC) | | transcarbamylase | ammonia buildup in blood |
| deficiency | | enzyme | (hyperammonemia) |
| Partington syndrome | ARX | Aristaless Related | Neurological disorder/development |
| | | Homeobox (ARX) | |
| Proud syndrome | ARX | Aristaless Related | Neurological disorder/development |
| | | Homeobox (ARX) | |
| Pyruvate dehydrogenase | PDHA1 | E1 alpha protein | Metabolic disorder; causes metabolic |
| complex (PDC) | | | and neurological dysfunction |
| deficiency | | | |
| Retinosis pigmentosa 2 | RP2 | | |
| (RP2) | | | |
| Retinosis pigmentosa 3 | RPGR | Retinitis pigmentosa | Severe form of inherited retinal |
| (RP3) | | GTPase regulator | degeneration, primarily affecting the |
| | | | rod photoreceptors; typically causes |
| | | | an early-onset night blindness and loss |
| | | | of peripheral vision, often causing |
| | | | blindness by the age of 30-40 years. |
| Retinosis pigmentosa 23 | OFD1 | | |
| (RP23) | | | |
| Snyder- | SMS | Spermine synthase | Condition characterized by |
| Robinson syndrome | | enzyme | intellectual disability and bone and |
| | | | muscle abnormalities |
| Spinal and bulbar | AR | Androgen receptor | Gradually progressive neuromuscular |
| muscular atrophy | | | disorder; degeneration of lower motor |
| (SBMA) or Kennedy's | | | neurons resulting in muscle weakness, |
| disease | | | muscle atrophy, and fasciculations; |
| | | | CAG trinucleotide repeat (>35 CAGs) |
| | | | in AR |
| Wilson-Tumer | HDAC8 | Histonedeacetylase 8 | Multisystem disorder |
| syndrome (WTS) | | | |
| Wiskott-Aldrich | WAS | WASP | Immune system disorder; primary T |
| syndrome | | | cell immunodeficiency |
| X-linked adrenal | NR0B1 | DAX1 | Characterized by adrenal |
| hypoplasia congenita | | | insufficiency, which may be life |
| | | | threatening, and hypogonadotropic |
| | | | hypogonadism |
| X-linked | ABCD1 | Adrenoleukodystrophy | Metabolic disorder |
| adrenoleukodystrophy | | protein (ALDP) | |
| (X-ALD) | | | |
| X-linked recessive | CMTX4: | CMTX4: Apoptosis- | Genetically heterogeneous group of |
| Charcot-Marie-Tooth | AIFM1 | inducing factor 1, | CMT peripheral sensorimotor |
| disease Types 4-5 | CMTX5: | mitochondrial; | polyneuropathy diseases; affects |
| (CMTX4-6) | PRPS1 | CMTX5: Ribose- | peripheral nerves; CMTXs are |
| | CMTX6: | phosphate | characterized by e.g., intellectual |
| | PDK3 | pyrophosphokinase 1; | deficit (CMTX2, CMTX4), spastic |
| | | CMTX6: Pyruvate | paraplegia (CMTX3), hearing loss |
| | | dehydrogenase kinase | (CMTX4, CMTX5, rarely CMTX1), |
| | | isozyme 3, | and optic atrophy (CMTX5) |
| | | mitochondrial | |
| X-linked creatine | SLC6A8 | Sodium- and chloride- | Development of the brain and nervous |
| deficiency | | dependent creatine | system; symptoms can include mild to |
| | | transporter 1 | severe intellectual disability, delayed |
| | | | speech development, behavioral |
| | | | problems, and seizures |
| X-linked dystonia- | TAF1 (or | TAF1 RNA polymerase | Neurodegenerative disorder |
| parkinsonism (XDP) | TAFII250) | II, TATA box- | |
| | | binding protein- | |
| | | associated factor, | |
| | | 250 kDa | |
| X-linked hypohidrotic | EDA | Ectodysplasin A | Genetic skin disease. Common |
| ectodermal dysplasia | | | symptoms include sparse scalp and |
| | | | body hair, reduced ability to sweat, |
| | | | and missing teeth |
| X-linked ichthyosis | STS | Steroid sulfatase | |
| (XLI) or steroid | | | |
| sulfatase deficiency; | | | |
| (SSD) | | | |
| X-linked | SH2D1A (XL | XLP1: Signaling | Immune system disorder |
| lymphoproliferative | P1) | lymphocyte activation | |
| disease (XLP; Types 1, | XIAP (XLP2) | molecule (SLAM) | |
| 2) | | associated protein | |
| | | (SAP); | |
| | | XLP2: XIAP protein | |
| X-Linked myopathy | VMA21 | Vacuolar ATPase | Muscle disease |
| with excessive | | assembly factor | |
| autophagy (XMEA) | | VMA21 | |
| X-linked myotubular | MTM1 | Myotubularin | Nervous system disorder; |
| myopathy (χLMTM) | | | centronuclear myopathy |
| X-linked recessive | DKC1 | Dyskerin | Telomere dysfunction |
| dyskeratosis congenita | | | |
| X-linked severe | IL2RG | ILR2 receptor, gamma | Immune system disorder |
| combined immuno- | | chain | |
| deficiency (X-SCID) | | | |
| X-linked sideroblastic | ALAS2 | 5′-aminolevulinate | Blood disease; metabolic disorder |
| anemia (XLSA) | | synthase 2 (erythroid | |
| | | specific, | |
| | | mitochondrial) | |
| X-linked | TRAPPC2 | Sedlin | Musculoskeletal disorder |
| spondyloepiphyseal | | | |
| dysplasia (SED) tarda | | | |
| X-linked | WAS | Wiskott-Aldrich | Blood disease |
| thrombocytopenia | | syndrome (WAS) | |
| (XLT) | | protein | |

TABLE 3

| Classified list | | Alphabet list | |
|---|---|---|---|
| Disease | Mutation | Disease | Mutation |
| Neurological diseases | | Adenocarcinoma | PPARγ |
| Epileptic spasms, West | ARX, CDKL5 | Adult neuronal ceroid | DNAJC5 |
| sydrome, | | lipofuscinosis (Parry | |
| | | disease) | |
| Early infantile epileptic | SCN8A, HCN1, | Agammaglobulinemia | E2A/TCF3 |
| encephalopathy (EIEE) | KCNA2, FGF12 | | |
| Autism | PTEN | Alzheimer's disease | PSEN1, APP |
| Familial amyotrophic | SOD1, C90rf72 | Apolipoprotein A-I | APOA1 |
| lateral sclerosis (fALS) | | deficiency | |
| Peripheral small-fiber | SCN9A(Nav1.7), | Atrial septal defect, ostium | TBX20 |
| neuropathy | SCN10A (Nav1.8) | secundum type | |
| KCNQ2-related | KCNQ2, KCNQ3 | Autosomal dominant | PCSK9 |
| encephalopathy | | hypercholesterolemia | |
| Down's syndrome | APP | Autosomal dominant | RHO |
| | | retinitis pigmentosa | |
| Parkinson disease | α-synuclein, LRRK1, 2 | Axenfeld-Rieger syndrome | PITX2 |
| GNB1 syndrom | GNB1 | Blackfan-Diamond anemia | RPL5, GATA1 |
| Holoproencephaly | SHH | Breast cancer | ATM |
| Tauopathy (demenita): AD, | MAPT | Carotid Intimal Medial | PPARγ |
| FTD, Pick disease (PiD), | | Thickness 1 | |
| progressive supranuclear | | | |
| palsy (PSP) and | | | |
| corticobasal degeneration | | | |
| (CBD) | | | |
| Alzheimer's disease | PSEN1, APP | Coloboma of choroid, | FZD5 |
| | | retina, eyelid, eye lens, iris | |
| | | and macula (Ocular | |
| | | coloboma) | |
| Huntington disease | Htt | Colon cancer, Familial | APC |
| | | adenomatous polyposis | |
| | | (FAP) | |
| Friedreich ataxia | FXN | Combined malonic and | ACSF3 |
| | | methylmalonic acidemia | |
| Spinocerebellar ataxia type | AFG3L2 | Cowden syndrome | PTEN |
| 28 | | | |
| Spinocerebellar ataxia type | ELOVL5 | Crouzon disease | FGFR2 |
| 38 | | (craniofacial dysostosis) | |
| Hereditary hyperekplexia | GLRA1, GLRB, and | Cystic Fibrosis | CFTR |
| | SLC6A5 | | |
| HSD10 disease | HSD17B10 | Cystinuria | SLC3A1, SLC7A9 |
| Encephalopathy due to | SLC2A1 | D-2-hydroxy glutaric | D2HGDH |
| GLUT1 deficiency | | aciduria | |
| Early Myoclonic | ErbB4 | Idiopathic dilated | PSEN1 |
| Encephalopathy (EME) | | cardiomyopathy (iDCM) | |
| Hereditary sensory and | HSN1 | Dominant optic atrophy | OPA1 |
| autonomic neuropathy type | | | |
| 1 | | | |
| Neuroferritinopathy | FTL | Dominant primary | FXYD2, HNF1 |
| | | hypomagnesemia with | |
| | | hypocalciuria | |
| Adult neuronal ceroid | DNAJC5 | Down's syndrome | APP |
| lipofuscinosis (Parry | | | |
| deisease) | | | |
| | | Early infantile epileptic | SCN8A, HCN1, |
| | | encephalopathy (EIEE) | KCNA2, FGF12 |
| Inflammatory and | | Early Myoclonic | ErbB4 |
| immune diseases | | Encephalopathy (EME) | |
| Hereditary pancreatitis | PRSS1 | Ehlers-Danlos syndrome | COL3A1 |
| | | type IV | |
| agammaglobulinemia | E2A/TCF3 | Encephalopathy due to | SLC2A1 |
| | | GLUT1 deficiency | |
| Hereditary angioedema | SERPING1 | Epileptic spasms, West | ARX, CDKL5 |
| (HAE) | | sydrome, | |
| Systemic mastocytosis | KIT | Erythropoietic | FECH, ALAS2 |
| | | protoporphyria | |
| Infantile enterocolitis | NLRC4 | Familial amyloid | TTR |
| | | cardiomyopathy (FAC) | |
| multi-organ epithelial | EGFR | Familial amyotrophic | SOD1, C90rf72 |
| inflammation | | lateral sclerosis (fALS) | |
| | | Familial atrial fibrillation | KCNQ1 |
| Muscular and bone | | Familiar colorectal cancer | SETD6 |
| disorders | | type X | |
| Presynaptic congenital | SLC5A7 | Familial gastrointestinal | KIT/PDGFRA |
| myasthenic syndromes | | stromal tumor | |
| Postsynaptic congenital | CHRNA1, | Familial juvenile | UMOD |
| myasthenic syndromes | CHRNDB1, CHRND, | hyperuricemic nephropathy | |
| | CHRNE | type 1 | |
| Limb-girdle muscular | myotilin | Familial primary | CNNM2, EGF |
| dystrophies LGMD1A | | hypomagnesemia with | |
| | | normocalciuria and | |
| | | normocalcemia | |
| Limb-girdle muscular | Lamin A/C | Familial progressive | SCN5A |
| dystrophies LGMD1B | | cardiac conduction defect | |
| Limb-girdle muscular | caveolin | Familial renal glucosuria | SLC5A2 |
| dystrophies LGMD1C | | | |
| osteogenesis imperfecta | COL1A1, COL1A2 | Familial thoracic aortic | ACTA2, TGFBR2 |
| (OI or brittle bone disease) | | aneurysm and aortic | |
| | | dissection (TAAD) | |
| GNE myopathy | GNE | Ferroportin-related | FTH1 |
| | | hemochromatosis (type 5) | |
| | | Friedreich ataxia | FXN |
| Cardiac and Vascular | | Genetic recurrent | MT-CO1, MT-CO2, |
| diseases | | myoglobinuria | LPIN1 |
| Familial progressive | SCN5A | GNB1 syndrome | GNB1 |
| cardiac conduction defect | | | |
| Romano-Ward syndrome | SCN5A, CAV3, | GNE myopathy | GNE |
| | SCN4B, SNTA1 | | |
| Familial atrial fibrillation | KCNQ1 | Glycogen storage disease | PHKA1, PHKA2, |
| | | due to liver or muscle | PHKB, PHKG |
| | | phosphorylase kinase | |
| | | deficiency (GSD-IX) | |
| Familial thoracic aortic | ACTA2, TGFBR2 | Hawkinsinuria | HPD |
| aneurysm and aortic | | | |
| dissection (TAAD) | | | |
| Atrial septal defect, ostium | TBX20 | Hemochromatosis type 4 | SLC40A1 |
| secundum type | | | |
| Ehlers-Danlos syndrome | COL3A1 | Hereditary angioedema | SERPING1 |
| type IV | | (HAE) | |
| Carotid Intimal Medial | PPARγ | Hereditary elliptocytosis | EOB41, SPTA1, |
| Thickness 1 | | | SPTB, SLC4A1 |
| diopathic dilated | PSEN1 | Hereditary | BCOM1 |
| cardiomyopathy (iDCM) | | hypercarotenemia and | |
| | | vitamin A deficiency | |
| Timothy syndrome | CACNA1C | Hereditary hyperekplexia | GLRA1, GLRB, and |
| | | | SLC6A5 |
| Familial amyloid | TTR | Hereditary pancreatitis | PRSS1 |
| cardiomyopathy (FAC) | | | |
| Apolipoprotein A-I | APOA1 | Hereditary sensory and | HSN1 |
| deficiency | | autonomic neuropathy | |
| | | type 1 | |
| | | Holoproencephaly | SHH |
| Retinal and | | HSD10 disease | HSD17B10 |
| ophthalmology diseases | | | |
| Isolated aniridia | PAX6 | Huntington disease | Htt |
| Coloboma of choroid, | FZD5 | Hyperinsulinism due to | GCK |
| retina, eyelid, eye lens, iris | | glucokinase deficiency | |
| and macula (Ocular | | | |
| coloboma) | | | |
| Stickler syndrome, retina | COL2A1 | Hyperinsulinism- | GLUD1 |
| lattice degeneration | | hyperammonemia | |
| | | syndrome | |
| Weill-Marchesani | fibrillin-1 (FBN1) | Infantile enterocolitis | NLRC4 |
| syndrome | | | |
| Autosomal dominant | RHO | Isolated aniridia | PAX6 |
| retinitis pigmentosa | | | |
| Late-onset retinal | CTRP5 | Isolated autosomal | KCNA1 |
| degeneration | | dominant | |
| | | hypomagnesemia, | |
| | | Glaudemans type | |
| Retinitis pigmentosa (form | PRPF31 | Isolated plagiocephaly, | FGFR3 |
| 11) | | brachycephaly, | |
| | | achondroplasia, | |
| | | hypochondroplasia, | |
| | | thanatophoric dysplasia | |
| | | types I and II | |
| Dominant optic atrophy | OPA1 | Juvenile cataract- | SLC16A12 |
| | | microcomea-renal | |
| | | glucosuria syndrome | |
| Progressive external | POLG, ANT1, | Juvenile granulosa cell | AKT1 |
| ophthalmoplegia | C10ORF2 | tumors | |
| Juvenile cataract- | SLC16A12 | KCNQ2-related | KCNQ2, KCNQ3 |
| microcornea-renal | | encephalopathy | |
| glucosuria syndrome | | | |
| | | Ketoacidosis due to | SLC16A1 |
| | | monocarboxylate | |
| | | transporter-1 deficiency | |
| Developmental diseases | | Late-onset retinal | CTRP5 |
| | | degeneration | |
| Noonan syndrome | TPN11, SOS1, RAF1, | Leukemia, acute | CEBPA |
| | and KRAS | myelogenous (AML) | |
| Crouzon disease | FGFR2 | L-Ferritin deficiency: (1) | FTL |
| (craniofacial dysostosis) | | hereditary | |
| | | hyperferritinemia with | |
| | | cataract syndrome (HHCS), | |
| | | (2) neuroferritinopathy, | |
| | | brain iron accumulation | |
| | | (NBIA), (3) | |
| | | hyperferritinemia, (4) L- | |
| | | ferritin deficiency | |
| Axenfeld-Rieger syndrome | PITX2 | Limb-girdle muscular | Myotilin |
| | | dystrophies LGMD1A | |
| Isolated plagiocephaly, | FGFR3 | Limb-girdle muscular | Lamin A/C |
| brachycephaly, | | dystrophies LGMD1B | |
| achondroplasia, | | | |
| hypochondroplasia, | | | |
| thanatophoric dysplasia | | | |
| types I and II | | | |
| Ketoacidosis due to | SLC16A1 | Limb-girdle muscular | Caveolin |
| monocarboxylate | | dystrophies LGMD1C | |
| transporter-1 deficiency | | | |
| X-linked dominant | EBP (X-linked) | Lipodystrophy, Familial | PPARγ |
| chondrodysplasia punctata | | Partial, Type 3 | |
| Blackfan-Diamond anemia | RPL5, GATA1 | Lipoprotein | APOE |
| | | glomerulopathy | |
| Sialuria | GNE | Marfan syndrome, | Fibrillin-1 (FBN10) |
| | | Marfanoid-progeroid- | |
| | | lipodystrophy syndrome, | |
| | | MASS syndrome, | |
| | | and Shprintzen-Goldberg | |
| | | syndrome | |
| Proteus syndrome | AKT1 | Methylmalonic acidemia | MMACHC, |
| | | with homocystinuria | MMADHC, LMBRD1, |
| | | | ABCD4 |
| | | Multi-organ epithelial | EGFR |
| | | inflammation | |
| Metabolism diseases | | Multiple osteochondromas | EXT1, EXT2 |
| Hemochromatosis type 4 | SLC40A1 | Neuroferritinopathy | FTL |
| L-ferritin deficiency: (1) | FTL | Noonan syndrome | TPN11, SOS1, RAF1, |
| hereditary | | | and KRAS |
| hyperferritinemia with | | | |
| cataract syndrome (HHCS), | | | |
| (2) neuroferritinopathy, | | | |
| brain iron accumulation | | | |
| (NBIA), | | | |
| (3)hyperferritinemia, (4) L- | | | |
| ferritin deficiency | | | |
| Autosomal dominant | PCSK9 | Osteogenesis imperfecta | COL1A1, C0L1A2 |
| hypercholesterolemia | | (OI or brittle bone disease) | |
| Lipodystrophy, Familial | PPARγ | Parkinson disease | α-synuclein, LRRK1, 2 |
| Partial, Type 3 | | | |
| Genetic recurrent | MT-CO1, MT-CO2, | Peripheral small-fiber | SCN9A(Nav1.7), |
| myoglobinuria | LPIN1 | neuropathy | SCN10A (Nav1.8) |
| Familial juvenile | UMOD | Phosphoserine | PSAT1 |
| hyperuricemic nephropathy | | aminotransferase | |
| type 1 | | deficiency (PSATD) | |
| Hereditary | BCOM1 | Postsynaptic congenital | CHRNA1, |
| hypercarotenemia and | | myasthenic syndromes | CHRNDB1, CHRND, |
| vitamin A deficiency | | | CHRNE |
| Isolated autosomal | KCNA1 | Progressive external | POLG, ANT1, |
| dominant | | ophthalmoplegia | C10ORF2 |
| hypomagnesemia, | | | |
| Glaudemans type | | | |
| Familial primary | CNNM2, EGF | Proteus syndrome | AKT1 |
| hypomagnesemia with | | | |
| normocalciuria and | | | |
| normocalcemia | | | |
| Dominant primary | FXYD2, HNF1 | Presynaptic congenital | SLC5A7 |
| hypomagnesemia with | | myasthenic syndromes | |
| hypocalciuria | | | |
| Ferroportin-related | FTH1 | Retinitis pigmentosa (form | PRPF31 |
| hemochromatosis (type 5) | | 11) | |
| Lipoprotein | APOE | Romano-Ward syndrome | SCN5A, CAV3, |
| glomerulopathy | | | SCN4B, SNTA1 |
| Transient neonatal multiple | ETFDH | Sialuria | GNE |
| acyl-CoA dehydrogenase | | | |
| deficiency | | | |
| Glycogen storage disease | PHKA1, PHKA2, | Spinocerebellar ataxia type | AFG3L2 |
| due to liver or muscle | PHKB, PHKG | 28 | |
| phosphorylase kinase | | | |
| deficiency (GSD-IX) | | | |
| Hyperinsulinism due to | GCK | Spinocerebellar ataxia type | ELOVL5 |
| glucokinase deficiency | | 38 | |
| Trehalase deficiency | TREH | Stickler syndrome, retina | COL2A1 |
| | | lattice degeneration | |
| Familial renal glucosuria | SLC5A2 | Systemic mastocytosis | KIT |
| Methylmalonic acidemia | MMACHC, | Tauopathy (demenita): AD, | MAPT |
| with homocystinuria | MMADHC, LMBRD1, | FTD, Pick disease (PiD), | |
| | ABCD4 | progressive | |
| | | supranuclear palsy (PSP) | |
| | | and corticobasal | |
| | | degeneration (CBD) | |
| Combined malonic and | ACSF3 | Timothy syndrome | CACNA1C |
| methylmalonic acidemia | | | |
| Phosphoserine | PSAT1 | Transient neonatal multiple | ETFDH |
| aminotransferase | | acyl-CoA dehydrogenase | |
| deficiency (PSATD) | | deficiency | |
| Hyperinsulinism- | GLUD1 | Trehalase deficiency | TREH |
| hyperammonemia | | | |
| syndrome | | | |
| D-2-hydroxyglutaric | D2HGDH | Weill-Marchesani | fibrillin-l (FBN1) |
| aciduria | | syndrome | |
| Cystinuria | SLC3A1, SLC7A9 | | |
| Hawkinsinuria | HPD | X-linked dominant | EBP (X-linked) |
| | | chondrodysplasia punctata | |
| Erythropoietic | FECH, ALAS2 | | |
| protoporphyria | | | |
| | | Glioblastoma, endometrial | PTEN |
| | | cancer, prostate, lung and | |
| | | breast cancer, Lhermitte- | |
| | | Duclos disease | |
| Other hereditary diseases | | 40% human tumors: | p53 |
| | | ovarian, esophageal, | |
| | | colorectal, head and neck, | |
| | | larynx, lung, skin, | |
| | | pancreas, stomach, liver, | |
| | | brain, bladder, breast, | |
| | | uterus, soft tissue, lymph | |
| | | nodes, prostate, bones, | |
| | | endocrine glads, hematop | |
| | | system, cervix | |
| Marfan syndrome, | Fibrillin-1 (FBN10) | Adenocarcinoma of the | EGFR |
| Marfanoid-progeroid- | | lung (40% of cases), anal | |
| lipodystrophy syndrome, | | cancers, glioblastoma (50%) | |
| MASS syndrome, | | and epithelian tumors of | |
| and Shprintzen-Goldberg | | the head and neck (80- | |
| syndrome | | 100%) | |
| Hereditary elliptocytosis | EOB41, SPTA1, | | |
| | SPTB, SLC4A1 | | |
| Cystic Fibrosis | CFTR | | |
| Tumor/cancer | | | |
| Familial gastrointestinal | KIT/PDGFRA | | |
| stromal tumor | | | |
| Cowden syndrome | PTEN | | |
| Juvenile granulosa cell | AKT1 | | |
| tumors | | | |
| Glioblastoma, endometrial | PTEN | | |
| cancer, prostate, lung and | | | |
| breast cancer, Lhermitte- | | | |
| Duclos disease | | | |
| 40% human tumors: | p53 | | |
| ovarian, esophageal, | | | |
| colorectal, head and neck, | | | |
| larynx, lung, skin, | | | |
| pancreas, stomach, liver, | | | |
| brain, bladder, breast, | | | |
| uterus, soft tissue, lymph | | | |
| nodes, prostate, bones, | | | |
| endocrine glads, hematop | | | |
| system, cervix | | | |
| Adenocarcinoma | PPARγ | | |
| Familiar colorectal cancer | SETD6 | | |
| type X | | | |
| Leukemia, acute | CEBPA | | |
| myelogenous (AML) | | | |
| Colon cancer, Familial | APC | | |
| adenomatous polyposis | | | |
| (FAP) | | | |
| Breast cancer | ATM | | |
| Adenocarcinoma of the | EGFR | | |
| lung (40% of cases), anal | | | |
| cancers, glioblastoma (50%) | | | |
| and epithelian tumors of | | | |
| the head and neck (80- | | | |
| 100%) | | | |
| Multiple osteochondromas | EXT1, EXT2 | | |

In another aspect, the present application provides a dual functional expression vector for developing an animal disease model in which the expression vector includes a nucleic acid suitably configured for expressing at least two active agents, where a first active agent is an siRNA/shRNA or miRNA for silencing an mRNA transcript expressed from a wild-type allele corresponding to a mutated allele known to cause an autosomal dominant, X-linked dominant or X-linked recessive genetic disease, where a second active agent is a mutant polypeptide corresponding to the mutated allele, and where the nucleic acid is genetically engineered to produce an mRNA transcript for the mutant polypeptide, wherein the mRNA transcript is insensitive to the silencing activity of the first active agent.

In one embodiment, the genetic disease is listed in Table 1 and the first and second active agents are directed to the corresponding gene or polypeptide associated therewith.

In another embodiment, the genetic disease is listed in Table 2 and the first and second active agents are directed to the corresponding gene or polypeptide associated therewith.

In another embodiment, the genetic disease is listed in Table 3 and the first and second active agents are directed to the corresponding gene or polypeptide associated therewith.

In a particular embodiment, the genetic disease is hereditary angioedema (HAE), the siRNA or miRNA is targeted for silencing a mutant C1 esterase inhibitor transcript, and the second active agent is a wild type C1 esterase inhibitor polypeptide.

In one embodiment, the animal models are humanized animals as important pre-clinical tools for studying and developing treatment of human infectious diseases.

In a particular embodiment, the human infectious disease is hepatitis B virus (HBV) infection, the siRNA or miRNA is targeted for silencing the mouse cellular sodium taurocholate co-transporting polypeptide (NTCP/SLC10A1), and the second active agent is the human counterpart NCTP polypeptide.

In another embodiment, the human infectious disease is coronavirus COVID-19 infection, the siRNA or miRNA is targeted for silencing the mouse angiotensin-converting enzyme 2 (ACE-2) as the entry receptor to infect host cells, and the second active agent is the human counterpart ACE2 polypeptide.

In one embodiment, the recombinant virus is formed from a recombinant virus vector derived from an adenovirus-associated virus (AAV), a retrovirus, a lentivirus or an adenovirus. In a particular embodiment, the recombinant virus is a recombinant AAV virus (rAAV).

In another aspect, the present application provides a method for developing an animal model for a genetic disease. The method includes the steps of: administering an effective amount of the aforementioned expression vector(s) in one or more animals; screening each of the one or more animals for expression of the mutant polypeptide and expression of a wild type transcript and/or the wild type polypeptide corresponding to the mutant polypeptide, and selecting an animal for use as a model for the particular genetic disease targeted by the expression vectors, which expressed the mutant polypeptide, but does not express the wild type transcript and/or the wild type polypeptide.

In another aspect, the present application provides an animal as a disease model for a particular disease targeted by the expression vectors above. In one embodiment, the animal model pertains to a genetic disease listed in Table 1 in which the first and second active agents are directed to the corresponding genetic disease in accordance with the aforementioned strategy. In another embodiment, the animal model pertains to a genetic disease listed in Table 2 in which the first and second active agents are directed to the corresponding genetic disease in accordance with the aforementioned strategy. In another embodiment, the animal model pertains to a genetic disease listed in Table 3 in which the first and second active agents are directed to the corresponding genetic disease in accordance with the aforementioned strategy.

### BRIEF DESCRIPTION OF THE DRAWINGS

FIG. 1 shows a 2-bullet-1-target strategy of dual functional expression vector for treatment of single gene hereditary diseases caused by autosomal dominant negative, X linked dominant, or X-linked recessive mutations.

FIG. 2 shows an exemplary vector design for treatment of HAE.

FIG. 3 shows an exemplary vector design for in vitro testing the efficacy of 2-bullet-1-target strategy for SERPING1 in human cells.

FIG. 4 shows the results of the expression of codon optimized human SERPING1-opt cDNA in human cells.

FIG. 5 shows the design and targets of siRNAs to knock-down all human SERPING1 disease-causing mutations.

FIG. 6, panels A-B show the results of screening of human SERPING1-targeted siRNA in HepG2 cells. The knock-down effect on SERPING1 mRNA level by qPCR (FIG. 6, panel A); and SERPING1 protein level by Western-blot (FIG. 6, panel B).

FIG. 7 shows the results of SERPING1 siRNA toxicity tests by cell proliferation in human cells.

FIG. 8 shows the results of efficacy of 2-bullet-1-target strategy for SERPING1 in human cells.

FIG. 9 shows an exemplary AAV vector design for in vivo efficacy testing of 2-bullet-1-target strategy for SERPING1 in mouse model.

FIG. 10 shows the results of the expression of codon optimized human SERPING1-opt cDNA in mouse.

FIG. 11 shows the design and targets of siRNAs for mouse SERPING1 gene.

FIG. 12 shows the results of knock-down effect of mouse SERPING1 siRNA in primary mouse hepatocytes.

FIG. 13, panels A-C show the results of efficacy of 2-bullet-1-target strategy to knock-down mouse SERPING1 without affecting codon optimized human SERPING1-opt cDNA expression in cells. Mouse SERPING1-targeted siRNA converted to shRNA (FIG. 13, panel A); Western-blot (FIG. 13, panel B) and quantification (FIG. 13, panel C) to detect the expression of mouse and human SEPRING1 in cells co-transfected with mouse shRNA and SERPING1 cDNA.

FIG. 14, panels A-B show the results of in vivo efficacy of 2-bullet-1-target strategy for SERPING1 in mouse. Mouse are injected intravenously through tail vein at various doses of “2 bullets” for SERPING1 on AAV8 vector. Expression of human SERPING1 protein level measured by ELISA in mouse plasma (FIG. 14, panel A); and mRNA level of mouse SERPING1 in mouse liver lysates (FIG. 14, panel B).

FIG. 15 shows an exemplary vector design for treatment of ALS.

FIG. 16 shows an exemplary vector design for in vitro efficacy testing of 2-bullet-1-target strategy for SOD1 in human cells.

FIG. 17 shows SOD1 siRNA construction strategy to knock-down all SOD1 disease-causing mutations.

FIG. 18, panels A-B show the results of screening of human SOD1-targeted siRNA in human cells. Western blot (FIG. 18, panel A) and quantification (FIG. 18, panel B) to detect the knock-down efficiency on endogenous SOD1 expression by siRNA.

### DETAILED DESCRIPTION OF THE INVENTION

The invention and accompanying drawings will now be discussed in reference to the numerals provided therein to enable one skilled in the art to practice the present invention. The skilled artisan will understand, however, that the inventions described below can be practiced without employing these specific details, or that they can be used for purposes other than those described herein. Indeed, they can be modified and can be used in conjunction with products and techniques known to those of skill in the art considering the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

It will be appreciated that reference throughout this specification to aspects, features, advantages, or similar language does not imply that all the aspects and advantages may be realized with the present invention should be or are in any single embodiment of the invention. Rather, language referring to the aspects and advantages is understood to mean that a specific aspect, feature, advantage, or characteristic described in connection with an embodiment is included in at least one embodiment of the present invention. Thus, discussion of the aspects and advantages, and similar language, throughout this specification may, but do not necessarily, refer to the same embodiment.

The described aspects, features, advantages, and characteristics of the invention may be combined in any suitable manner in one or more further embodiments. Furthermore, one skilled in the relevant art will recognize that the invention may be practiced without one or more of the specific aspects or advantages of a particular embodiment. In other instances, additional aspects, features, and advantages may be recognized and claimed in certain embodiments that may not be present in all embodiments of the invention.

Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### I. Definitions

As used in this specification and the appended claims, the singular forms “a”, “an” and “the” include plural referents unless the content clearly dictates otherwise.

Further, where the phrases “in one embodiment”, “in another embodiment” “in other embodiments”, “in some embodiments” or “in certain embodiments” are used, the present disclosure should be construed as embracing combinations of any of the features defining the different embodiments described therein, unless the features are not combinable with one another, are mutually exclusive, or are expressly disclaimed herein.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” (i.e. the value), “greater than or equal to” (i.e. the value) and possible ranges between values are also disclosed, as appropriately understood by the skilled artisan. For example, if the value “10” is disclosed the “less than or equal to 10” as well as “greater than or equal to 10” is also disclosed.

As used herein, the term “agent” is used with reference to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

The term “allele” as used hereinafter, refers to one of two or more versions of a known gene located at the same place on a chromosome (e.g., various forms of the same gene including mutated forms of a wild-type gene). It can also refer to the same gene in different species (e.g., the mouse allele of the human ACE-2 gene). Alleles can come in different extremes of size. At the lowest possible end one can be the single base choice of a single nucleotide polymorphism (SNP). At the higher end, it can be the sequence variations for the regions of the genome that code for the same protein which can be up to several thousand base-pairs long.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments.

As used herein, the term “isolated” means: 1) separated from at least some of the components with which it is usually associated in nature; 2) prepared or purified by a process that involves the hand of man; and/or 3) not occurring in nature, e.g., present in an artificial environment. By “isolated,” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition. In some embodiments an isolated cell is a cell that has been removed from a subject, generated in vitro, separated from at least some other cells in a cell population or sample, or that remains after at least some other cells in a cell population or sample have been removed or eliminated.

As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate.

As used herein, the term “nucleic acid” relates to a DNA or RNA molecule. The nucleic acid molecule may be derived from a variety of sources, including DNA, cDNA, synthetic DNA, RNA or combinations thereof. Such nucleic acid sequences may comprise genomic DNA which may or may not include naturally occurring introns. Moreover, such genomic DNA may be obtained in association with promoter regions, poly A sequences or other associated sequences. Genomic DNA may be extracted and purified from suitable cells by means well known in the art. Alternatively, messenger RNA (mRNA) can be isolated from cells and used to produce cDNA by reverse transcription or other means.

As used herein, the term “autosomal dominant” is used with reference to a pattern of inheritance characteristic of certain genetic diseases caused by a mutated “autosomal” gene located on a non-sex chromosome. In this context, the term “dominant” means that a mutation in a single copy (or allele) of the disease-associated autosomal gene is enough to cause the disease.

As used herein, the term “X-linked dominant” is used with reference to a pattern of inheritance characteristic of certain genetic diseases caused by a mutated gene located on an allele of the X chromosome (a sex chromosome), where “dominant” means that a mutation in at least one copy (or allele) of the disease-associated X chromosomal gene enough to cause the disease.

As used herein, the term “X-linked recessive” is used with reference to a pattern of inheritance characteristic of certain genetic diseases caused by a mutated gene located on an allele of the X chromosome, where “recessive” means that a mutation in one copy (or allele) of the disease-associated X chromosomal gene is insufficient to substantially cause the disease in a female carrying two copies of the X chromosome, but is sufficient to cause the disease in a male having just one copy of the X chromosome.

As used herein, the phrase, “gain of function mutation” refers to a mutation resulting in the acquisition of a new, altered, and/or abnormal function or increased function as compared with a reference. The reference may be, e.g., a level or average level of function possessed by a normal gene product (e.g., a gene product whose sequence is the same as a reference sequence) or found in healthy cell(s) or subject(s). An average may be taken across any number of values. In certain embodiments, the reference level may be the upper limit of a reference range. In certain embodiments the function may be increased by at least 10%, 20%, 30%, 40%, 50%, 60%, 70%, 80%, 90%, 95%, 99%, or 100% of the reference level. In certain embodiments the function may be increased by between 1 to 2-fold, 2 to 5-fold, 5 to 10-fold, 10 to 20-fold, 20 to 50-fold, 50 to 100-fold, or more, of the reference level. In certain embodiments the function may be increased to a level or within a range that has a statistically significant correlation with or demonstrated causative relationship with a neurodegenerative disease.

A “gain of function” mutation in a gene results in a change in a gene product of the gene or increases the expression level of the gene product, such that it gains a new and abnormal function or an abnormally increased function as compared with a gene product of a normal gene. The function may be new in that it is distinct from the activit(ies) of the normal gene product or may result from an increase in or dysregulation of a normal activity of the gene product. The altered gene product encoded by a gene harboring a gain of function mutation may, for example, have one or more altered residues that causes the gene product to have the ability to interact with different cellular molecules or structures than does the normal gene product or causes the gene product to be mislocalized or dysregulated. For purposes hereof, gain of function mutations encompass dominant negative mutations. In some embodiments a phenotype or disease resulting from a gain of function mutation in a diploid cell or organism has an autosomal dominant inheritance pattern. A “function” may be any biological activity of a gene product. A biological activity may be, for example, catalyzing a particular reaction, binding to or transporting a particular molecule or complex, participating in or interfering with a biological process carried out by a cell or cells or within a subject, etc. The particular function(s) resulting from a gain of function mutation or lost due to a loss of function mutation may or may not be known.

The phrase, “loss of function mutation” is used herein with reference to a mutation resulting in a reduction of function or absence of function as compared with a reference level. The reference level may be, e.g., a normal or average level of function possessed by a normal gene product or found in a healthy cell or subject. In certain embodiments the reference level may be the lower limit of a reference range. In certain embodiments the function may be reduced by at least 10%, 20%, 30%, 40%, 50%, 60%, 70%, 80%, 90%, 95%, 99%, or 100% of the reference level.

A “loss of function” mutation in a gene refers to a mutation that causes loss (reduction or absence) of at least one function normally provided by a gene product of the gene. A loss of function mutation in a gene may result in a reduced total level of a gene product of the gene in a cell or subject that has the mutation (e.g., due to reduced expression of the gene, reduced stability of the gene product, or both), reduced activity per molecule of the gene product encoded by the mutant gene, or both. The reduction in expression, level, activity per molecule, or total function may be partial or complete. A mutation that confers a complete loss of function, or an allele harboring such a mutation, may be referred to as a null mutation or null allele, respectively. In some embodiments a loss of function mutation in a gene results in a reduction of at least 10%, 20%, 30%, 40%, 50%, 60%, 70%, 80%, 90%, 95%, 99%, or 100% in the level or activity of a gene product of the mutant gene, as compared with level or activity of a gene product encoded by a normal allele of the gene.

A loss of function mutation may be an insertion, deletion, or point mutation. For example, a point mutation may introduce a premature stop codon, resulting in a truncated version of the normal gene product that lacks at least a portion of a domain that contributes to or is essential for activity, such as a catalytic domain or binding domain, or may alter an amino acid that contributes to or is essential for activity, such as a catalytic residue, site of post-translational modification, etc. In some embodiments a phenotype or disease resulting from a loss of function mutation in a diploid cell or organism has an autosomal recessive inheritance pattern.

As used herein, the phrase “dominant negative mutation” (also called antimorphic mutations) is used with reference to a mutation resulting in an altered gene product that acts antagonistically to the wild-type allele. These mutations usually result in an altered molecular function (often inactive) and are characterized by a dominant or semi-dominant phenotype. The altered gene product associated with a dominant negative mutations may act antagonistically to the normal gene product by, for example, by competing with the normal gene product in a context such as a binding partner, ligand, component of a multimolecular complex (e.g., an oligomer), or substrate such that it fails to fulfill the normal function of the gene product in that context. The altered gene product encoded by a gene harboring a dominant negative mutation may, for example, be a truncated or otherwise altered form of the normal gene product that retains sufficient structure to compete with the normal gene product. In humans, dominant negative mutations have been implicated in cancer (e.g., mutations in genes p53, ATM, CEBPA, and PPARgamma. Marfan syndrome is caused by dominant negative mutations in the FBN1 gene (coding for fibrillin-1, a glycoprotein component of the extracellular matrix) further resulting in haploinsufficiency.

As used herein, the phrase “humanized animal model” is used with reference to pre-clinical experimental animals (mouse, rat, pig and nonhuman primates) that carry human DNA and/or express human proteins.

A “reference range” for a value, e.g., a reference range for a value associated with a gene product, biological activity, cell, or subject, refers to the range into which 95%, or in some embodiments 90%, of the values measured from normal or control gene products or healthy cells or subjects fall, or a range that encompasses only values that do not have a statistically significant correlation with a type of disease in general or with a particular disease of interest as compared to the average value in healthy cells or subjects. A reference range may be established from a representative sample of a population. In some embodiments a reference range may be established by performing measurements on gene products or healthy cells obtained from multiple subjects who are apparently healthy or at least free of a particular disease type or disease of interest and not known to be at increased risk of developing the disease.

As used herein, the term “heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector. As an example, “heterologous” when used in the context of nucleic acid sequences, such as coding sequences and control sequences, may refer to sequences that are not normally joined together, and/or are not normally associated with one another under ordinary circumstances found in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this application. Allelic variation or naturally occurring mutational events do not give rise to heterologous DNA, as used herein.

A “coding sequence” refers to a nucleic acid sequence which “encodes” a particular protein. The nucleic acid sequence in a polynucleotide is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence is usually be located 3′ to the coding sequence.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter region” is used herein in its ordinary sense to refer to a DNA regulatory sequence to which RNA polymerase binds, initiating transcription of a downstream (3′ direction) coding sequence. Further, the term should be broadly construed as additionally encompassing other regulatory elements, including enhancer regions, intron splice donors and acceptors, other 5′ untranslated regions and the like. A promoter sequence may be homologous or heterologous to the desired gene sequence. A wide range of promoters are known and available in the art for the present application, including a wide range of viral and mammalian promoters. Cell type selective or tissue specific promoters can be utilized to target or enhance expression of gene sequences in specific cell populations relative to others. Suitable mammalian and viral promoters. A promoter may be constitutively active, conditionally active, or inducible, depending on the cell type.

“Operably linked” refers to an arrangement of elements wherein the components so described are configured so as to perform their usual function. Thus, control sequences operably linked to a coding sequence are capable of effecting the expression of the coding sequence. Operably linking a heterologous sequence to a promoter, results in a chimeric gene. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of a nucleotide sequence in a particular nucleic acid molecule throughout the present application, such as when a particular nucleotide sequence is described as being situated “upstream”, “downstream”, “3′”, or “5′” relative to another sequence, these modifiers should be construed as relating sequence portions in the “sense” or “coding” strand of a DNA molecule, as is conventional in the art.

The term “transgene” refers to a polynucleotide that is introduced into a cell and is capable of being transcribed into RNA and optionally, translated and/or expressed under appropriate conditions. The transgene confers a desired property to a cell into which it was introduced, or otherwise leads to a desired therapeutic or diagnostic outcome. In another aspect, it may be transcribed into a molecule that mediates RNA interference, such as an miRNA, an siRNA, an shRNA, or a guide RNA for CRISPR/Cas9 mediated targeting of the mutant allele.

The term “expression” encompasses the processes by which nucleic acids (e.g., DNA) are transcribed to produce RNA, and (where applicable) RNA transcripts are processed and translated into polypeptides.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (1): 308-311; Kitts A, and Sherry S, (2009). Multiple isoforms of certain proteins may exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments may be directed to particular sequence(s), e.g., particular allele(s) or isoform(s).

As used herein, the term “RNA interference” or “RNAi” refers generally to RNA-dependent silencing of gene expression initiated by double stranded RNA (dsRNA) molecules in a cell's cytoplasm. dsRNA molecule reduces or inhibits transcription products of a target nucleic acid sequence, thereby silencing the gene or reducing expression of that gene.

As used herein, the term “double stranded RNA” or “dsRNA” refers to a RNA molecule having a duplex structure and comprising an effector sequence and an effector complement sequence which are of similar length to one another. The effector sequence and the effector complement sequence can be in a single RNA strand or in separate RNA strands. The “effector sequence” (often referred to as a “guide strand”) is substantially complementary to a mutant target sequence targeted by the first active agent. The “effector sequence” can also be referred to as the “antisense sequence”. The “effector complement sequence” will be of sufficient complementary to the effector sequence such that it can anneal to the effector sequence to form a duplex. In this regard, the effector complement sequence will be substantially homologous to a region of target sequence. As will be apparent to the skilled person, the term “effector complement sequence” can also be referred to as the ‘complement of the effector sequence” or the sense sequence.

As used herein, the term “duplex” refers to regions in two complementary or substantially complementary nucleic acids (e.g., RNAs), or in two complementary or substantially complementary regions of a single-stranded nucleic acid (e.g., RNA), that form base pairs with one another, either by Watson-Crick base pairing or any other manner that allows for a stabilized duplex between the nucleotide sequences that are complementary or substantially complementary. It will be understood by the skilled person that within a duplex region, 100% complementarity is not required; substantial complementarity is allowable. Substantial complementarity may include, for example, 79% or greater complementarity. For example, a single mismatch in a duplex region consisting of 19 base pairs (i.e., 18 base pairs and one mismatch) results in 94.7% complementarity, rendering the duplex region substantially complementary. In another example, two mismatches in a duplex region consisting of 19 base pairs (i.e., 17 base pairs and two mismatches) results in 89.5% complementarity, rendering the duplex region substantially complementary. In yet another example, three mismatches in a duplex region consisting of 19 base pairs (i.e., 16 base pairs and three mismatches) results in 84.2% complementarity, rendering the duplex region substantially complementary, and so on.

The dsRNA may be provided as a hairpin or stem loop structure, with a duplex region comprised of an effector sequence and effector complement sequence linked by at least 2 nucleotide sequence which is termed a stem loop. When a dsRNA is provided as a hairpin or stem loop structure it can be referred to as a “hairpin RNA” or “short hairpin RNAi agent” or “shRNA”. Other dsRNA molecules provided in, or which give rise to, a hairpin or stem loop structure include primary miRNA transcripts (pri-miRNA) and precursor microRNA (pre-miRNA). Pre-miRNA shRNAs can be naturally produced from pri-miRNA by the action of the enzymes Drosha and Pasha which recognize and release regions of the primary miRNA transcript which form a stem-loop structure. Alternatively, the pri-miRNA transcript can be engineered to replace the natural stem-loop structure with an artificial/recombinant stem-loop structure. That is, an artificial/recombinant stem-loop structure may be inserted or cloned into a pri-miRNA backbone sequence which lacks its natural stem-loop structure. In the case of stem-loop sequences engineered to be expressed as part of a pri-miRNA molecule, Drosha and Pasha recognize and release the artificial shRNA. dsRNA molecules produced using this approach are known as “shmiRNAs”, “shmiRs” or “microRNA framework shRNAs”.

As used herein, the term “siRNA” is used with reference to short (20-25 nucleotides), double-stranded RNA molecules that are engineered to use the RNAi pathway to degrade a target mRNA to induce sequence-specific post-transcriptional gene silencing of mRNAs. Synthetically produced siRNAs structurally mimic the types of siRNAs normally processed in cells by the enzyme Dicer. When expressed from a viral vector, the viral vector is engineered to transcribe a short double-stranded hairpin-like RNA (shRNA) that is processed into a targeted siRNA inside the cell. Upon delivery into the cytoplasm, argonaute (AGO)2 cleaves the passenger (sense) strand and the guide (antisense) strand of the siRNA is loaded into the RNA-induced silencing complex (RISC). The guide strand then guides the RISC to the target mRNA which is recognized and cleaved. The RISC and guide strand can be recycled and therefore one siRNA molecule can drive the cleavage of multiple mRNA molecules resulting in highly efficient gene silencing.

As used herein, the term “DNA-directed RNAi construct” or “ddRNAi construct” refers to a nucleic acid comprising DNA sequence which, when transcribed produces a shRNA or shmiR molecule (preferably a shmiR) which elicits RNAi. The ddRNAi construct may comprise a nucleic acid which is transcribed as a single RNA that is capable of self-annealing into a hairpin structure with a duplex region linked by a stem loop of at least 2 nucleotides i.e., shRNA or shmiR, or as a single RNA with multiple shRNAs or shmiRs, or as multiple RNA transcripts each capable of folding as a single shRNA or shmiR respectively. The ddRNAi construct may be provided within a larger “DNA construct” comprising one or more additional DNA sequences. For example, the ddRNAi construct may be provided in a DNA construct further comprising a DNA sequence coding for functional wild type allele which has been genetically altered so that its mRNA transcript is not targeted by RNAi in the ddRNAi construct.

As used herein, the term “complementary” with regard to a sequence refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence.

As used herein, the term “substantially complementary” is used to indicate a sufficient degree of complementarity or precise pairing such that stable and specific binding occurs between nucleic acid sequences e.g., between the effector sequence and the effector complement sequence or between the effector sequence and the target sequence. It is understood that the sequence of a nucleic acid need not be 100% complementary to that of its target or complement. The term encompasses a sequence complementary to another sequence with the exception of an overhang. Depending on the length of the area of complementarity, a sequence may be complementary to another sequence with the exception of 1 mismatch, 2 mismatches, 3 mismatches, 4 mismatches, 5 mismatches, 6 mismatches, 7 mismatches, 8 mismatches, 9 mismatches, 10 mismatches or more.

The term “encoded”, as used in the context of an shRNA or shmiR of the present application, shall be understood to mean a shRNA or shmiR which is capable of being transcribed from a DNA template. Accordingly, a nucleic acid that encodes, or codes for, a shRNA or shmiR of the present application will include a DNA sequence that serves as a template for transcription of the respective shRNA or shmiR.

As used herein, the terms “transfection”, “gene transfer”, “gene delivery” are used interchangeably herein with reference to methods or systems for inserting foreign nucleic acids into host cells. Gene transfer can result in transient expression of non-integrated transferred DNA, extrachromosomal replication and expression of transferred replicons (e.g., episomes), or integration of transferred genetic material into the genomic DNA of host cells. Various techniques are known to those of ordinary skill in the art to introduce one or more exogenous nucleic acid molecules, into suitable host cells, including chemical, electrical, and viral-mediated transfection procedures.

The term “gene therapy” is used herein with reference to treatment of a disease or disorder by introducing a suitable polynucleotide into cells in vivo or ex vivo.

The term “host cell” as used herein generally refers to a cell (e.g., bacterial cell, yeast cell, insect cell, mammalian cell) which serves as a recipient for exogenously introduced nucleic acids or has been transfected with an exogenous nucleic acid. It is understood that the progeny of a single parental cell may not necessarily be completely identical in morphology or in genomic or total DNA complement to the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

A nucleotide or amino acid residue in a first nucleic acid or protein “corresponds to” a residue in a second nucleic acid or protein if the two residues perform one or more corresponding functions and/or are located at corresponding positions in the first and second nucleic acids or proteins. Corresponding functions are typically the same, equivalent, or substantially equivalent functions, taking into account differences in the environments of the two nucleic acids or proteins as appropriate. Residues at corresponding positions typically align with each other when the sequences of the two nucleic acids or proteins are aligned to maximize identity (allowing the introduction of gaps) using a sequence alignment algorithm or computer program such as those referred to below (see “Identity”) and/or are located at positions such that when the 3-dimensional structures of the proteins is superimposed the residues overlap or occupy structurally equivalent positions and/or form the same, equivalent, or substantially equivalent intramolecular and/or intermolecular contacts or bonds (e.g., hydrogen bonds). The structures may be experimentally determined, e.g., by X-ray crystallography or NMR or predicted, e.g., using structure prediction or molecular modeling software. An alignment may be over the entire length of one or more of the aligned nucleic acid or polypeptide sequences or over at least one protein domain (or nucleotide sequence encoding a protein domain).

A “domain” of a protein is a distinct functional and/or structural unit of a protein, e.g., an independently folding unit of a polypeptide chain. In some embodiments a domain is a portion of a protein sequence identified as a domain in the Conserved Domain Database of the NCBI (Marchler-Bauer A et al. (2013), “CDD: conserved domains and protein three-dimensional structure”, Nucleic Acids Res. 41(D1):D384-52). In some embodiments corresponding amino acids are the same in two sequences (e.g., a lysine residue, a threonine residue) or would be considered conservative substitutions for each other. Examples of corresponding residues include (i) the catalytic residues of two homologous enzymes and (ii) sites for post-translational modification of a particular type (e.g., phosphorylation) within corresponding structural or functional domains that have similar effects on the structure or function of homologous proteins.

“Homology” refers to the percent of identity between two polynucleotide or two polypeptide moieties. The correspondence between a polynucleotide sequence from one moiety to another can be determined by techniques known in the art. For example, homology can be determined by a direct comparison of the sequence information between two polypeptide molecules by aligning the sequence information and using readily available computer programs to evaluate the extent of homology. Alternatively, homology can be determined by hybridization of polynucleotides under conditions which allow for the formation of stable duplexes between homologous regions, followed by digestion with single stranded-specific nuclease(s), and size determination of the digested fragments. Two DNA, or two polypeptide sequences are “substantially homologous” to each other when at least about 80%, preferably at least about 90%, and most preferably at least about 95%, at least about 96%, at least about 97%, at least about 98%, or at least about 99% of the nucleotides or amino acids match over a defined length of the molecules, as determined using the methods above.

A “functional homologue” or a “functional equivalent” of a given polypeptide includes molecules derived from the native polypeptide sequence, as well as recombinantly produced or chemically synthesized polypeptides which function in a manner similar to the reference molecule to achieve a desired result.

A “variant” of a particular polypeptide or polynucleotide has one or more additions, substitutions, and/or deletions with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. A “vector” is used herein with reference to a recombinant plasmid or virus that includes a heterologous nucleic acid of interest to be delivered into a host cell, either in vitro or in vivo. The nucleic acid of interest may be linked to, e.g., inserted into, the vector using, e.g., restriction and ligation. Vectors include, for example, DNA or RNA plasmids, cosmos, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors, which are further described below. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed.

An “expression vector” is a vector designed to incorporate a desired nucleic acid of interest in operable linkage to regulatory elements (also termed “regulatory sequences”, “expression control elements”, or “expression control sequences”) mediating expression of the nucleic acid of interest as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. Expression vectors include non-viral vectors such as plasmid vectors, and viral vectors such as adeno virus vectors, adeno-associated virus (AAV) vectors, lentivirus vectors, herpes virus vectors.

As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1α, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase I promoter, e.g., a promoter for transcription of ribosomal RNA (other than 5S rRNA) or a functional variant thereof is used. In some embodiments, a promoter that ordinarily directs transcription by a eukaryotic RNA polymerase III (a “pol III promoter”), e.g., (a U6, H1, 7SK or tRNA promoter or a functional variant thereof) may be used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEF1 vector series, Gateway® vectors, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen, N, et al. (2000) Mouse Genetics and Transgenics: 259-263; Urlinger, S, et al. (2000). Proc. Natl. Acad. Sci. U.S.A. 97 (14): 7963-8; Zhou, X., et al (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof, metal-regulated systems (e.g., metallothionein promoter) may be used in certain embodiments. In some embodiments, tissue-specific or cell type specific regulatory element(s) may be used, e.g., in order to direct expression in one or more selected tissues or cell types. In some embodiments a vector capable of being stably maintained and inherited as an episome in mammalian cells (e.g., an Epstein-Ban virus-based episomal vector) may be used.

As used herein, the term “viral vector” refers to a recombinant polynucleotide vector comprising virally-derived nucleic acids containing sequences facilitating replication and expression of exogenously incorporated transgene sequences operatively linked to suitable control elements and one or more heterologous sequences (i.e., nucleic acid sequence not of viral origin).

The term “recombinant virus” is used herein with reference to a virus that has been genetically altered, e.g., by the addition or insertion of a heterologous nucleic acid construct into a virus particle.

The term “parvovirus” refers to a DNA animal virus that contains a linear, single-stranded DNA genome, which is classified in the Parvoviridae family, and includes autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, feline, panleukopenia virus, feline parvovirus, goose parvovirus, and B19 virus. Other autonomous parvoviruses are known to those skilled in the art.

The dependovirus genus includes adeno-associated viruses (AAV), including but not limited to, AAV type 1, AAV type 2, AAV type 3, AAV type 4, AAV type 5, AAV type 6, avian AAV, bovine AAV, canine AAV, equine AAV, and ovine AAV. See, e. g., Bernard N. Fields et al., Virology, vol. 2, ch. 69 (3d ed., Lippincott-Raven Publishers).

The term “wild-type AAV” as used herein refers to both wild-type and pseudo-wild-type AAV. “Pseudo-wild-type AAV” are replication-competent AAV virions produced by either homologous or non-homologous recombination between an AAV vector carrying ITRs and an AAV helper vector carrying rep and cap genes. Pseudo-wild-type AAV have nucleic acid sequences that differ from wild-type AAV sequences.

By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, i.e., “sense” or “antisense” strands, can be packaged into any one AAV virion and both strands are equally infectious.

The terms “AAV vector” and “recombinant AAV vector (rAAV vector)” are used interchangeably herein with reference to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

An “AAV vector” may be derived from any adeno-associated virus serotype, including without limitation, AAV-1, AAV-2, AAV-3, AAV-4, AAV-5, AAV-6, AAV-7, AAV-8, AAV-9 etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The terms “recombinant AAV virion,” “rAAV virion”, and “rAAV virus particle” is used interchangeably herein with reference to an infectious, replication-defective virus particle composed of viral particle composed of at least one AAV capsid protein and an encapsidated rAAV vector genome comprising a heterologous nucleotide sequence of interest that is flanked on both sides by AAV ITRs. A rAAV virion is produced in a suitable host cell comprising an AAV vector, AAV helper functions, and accessory functions. A host cell containing these components is capable of encoding AAV polypeptides required for packaging the AAV vector (containing a recombinant nucleotide sequence of interest) into infectious recombinant virion particles for subsequent gene delivery.

By “adeno-associated virus inverted terminal repeats” or “AAV ITRs” is meant the art-recognized regions found at each end of the AAV genome which function together in cis as origins of DNA replication and as packaging signals for the viral genome. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, R. M. (1994) Human Gene Therapy 5, 793-801; Bems, K. I. “Parvoviridae and their Replication” In Fundamental Virology, 2d ed., (B. N. Fields and D. M. Knipe, eds.) for the AAV-2 sequence. As used herein, an “AAV ITR” need not have the wild-type nucleotide sequence depicted in the previously cited references, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV-6, AAV-7, AAV-8, AAV-9 etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

By “AAV rep coding region” is meant the art-recognized region of the AAV genome which encodes the replication proteins of the virus which are required to replicate the viral genome and to insert the viral genome into a host genome during latent infection. The term also includes functional homologues thereof such as the human herpesvirus 6 (HHV-6) rep gene which is also known to mediate AAV-2 DNA replication (Thomson et al. (1994) Virology 204, 304-311). For a further description of the AAV rep coding region, see, e.g., Muzyczka, N. (1992) Current Topics in Microbiol. and Immunol. 158, 97-129; Kotin, R. M. (1994) Human Gene Therapy 5, 793-801. The rep coding region, as used herein, can be derived from any viral serotype, such as the AAV serotypes described above. The region need not include all of the wild-type genes but may be altered, e.g., by the insertion, deletion or substitution of nucleotides, so long as the rep genes present provide for sufficient integration functions when expressed in a suitable recipient cell.

The term “long forms of Rep” refers to the Rep 78 and Rep 68 gene products of the AAV rep coding region, including functional homologues thereof. The long forms of Rep are normally expressed under the direction of the AAV p5 promoter.

The term “AAV producer cell” refers to a mammalian or insect cell that can support AAV production.

The phrase “short forms of Rep” refers to the Rep 52 and Rep 40 gene products of the AAV rep coding region, including functional homologues thereof. The short forms of Rep are expressed under the direction of the AAV p19 promoter.

By “AAV cap coding region” is meant the art-recognized region of the AAV genome which encodes the coat proteins of the virus which are required for packaging the viral genome. For a further description of the cap coding region, see, e.g., Muzyczka, N. (1992) Current Topics in Microbiol. and Immunol. 158, 97-129; Kotin, R. M. (1994) Human Gene Therapy 5, 793-801. The AAV cap coding region, as used herein, can be derived from any AAV serotype, as described above. The region need not include all of the wild-type cap genes but may be altered, e.g., by the insertion, deletion or substitution of nucleotides, so long as the genes provide for sufficient packaging functions when present in a host cell along with an AAV vector.

“AAV helper functions” refer to AAV-derived coding sequences that can be expressed to provide AAV gene products that, in turn, function in trans for productive AAV replication. Thus, AAV helper functions include the rep and cap regions. The rep expression products have been shown to possess many functions, including, among others: recognition, binding and nicking of the AAV origin of DNA replication; DNA helicase activity; and modulation of transcription from AAV (or other heterologous) promoters. The cap expression products supply necessary packaging functions. AAV helper functions are used herein to complement AAV functions in trans that are missing from AAV vectors.

The term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virology 63, 3822-3828; McCarty et al. (1991) J. Virology 65, 2936-2945. A number of other vectors have been described which encode Rep and/or Cap expression products. See, e.g., U.S. Pat. No. 5,139,941.

The term “accessory functions” refers to non-AAV derived viral and/or cellular functions upon which AAV is dependent for its replication. Thus, the term embraces DNAs, RNAs and protein that are required for AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

Adenovirus-derived accessory functions have been widely studied, and a number of adenovirus genes involved in accessory functions have been identified and partially characterized. Specifically, early adenoviral E1A, E1B 55K, E2A, E4, and VA RNA gene regions are thought to participate in the accessory process. Janik et al. (1981) Proc. Natl. Acad. Sci. USA 78, 1925-1929. Herpesvirus-derived accessory functions have been described. See, e.g., Young et al. (1979) Prog. Med. Virol. 25, 113. Vaccinia virus-derived accessory functions have also been described. See, e.g., Carter, B. J. (1990), supra., Schlehofer et al. (1986) Virology 152, 110-117.

The term “encapsidation essential adenoviral gene product” refers to an adenoviral gene product essential for encapsidating an adenoviral genome to produce infectious adenovirus particles. Encapsidation typically include “late phase” adenoviral gene products. Exemplary encapsidation essential adenoviral gene products include capsid protein IX, encapsidation protein IVa2, protein 13.6, encapsidation protein 52K, capsid protein precursor pIIIa, penton base (capsid protein III), core protein precursor pVII, core protein V, core protein precursor pX, capsid protein precursor pVI, hexon (capsid protein II), protease, hexon assembly protein 100K, protein 33K, encapsidation protein 22K, capsid protein precursor pVIII, protein UXP, and fiber (capsid protein IV).

The term “encapsidation non-essential adenoviral gene product” refers to an adenoviral gene product that is dispensable for encapsidating an adenoviral genome to produce infectious adenovirus particles. Encapsidation non-essential adenoviral gene products are typically translated from adenovirus E1, E2, E3 and E4 transcriptional units. Thus, encapsidation non-essential adenoviral gene products may include gene products expressed from the adenoviral E1 transcriptional unit (e.g., E1A, E1B-19K, E1B-55K); gene products the adenoviral E2/E2A transcriptional unit (e.g., Iva2, pol, pTP, DBP); gene products the adenoviral E3 transcriptional unit (e.g., E3 CR1 alpha0, E3 gp19, E3 14.7 K, E3 RID-beta); gene products from the E4 transcriptional unit (e.g., E4 34K, E4 ORF1, E4 ORFB, E4 ORF3, E4 ORF4, E4 ORF6/7); or a combination thereof.

A “functional homolog” or a “functional equivalent” of a given adenoviral nucleotide region includes similar regions derived from a heterologous adenovirus serotype, nucleotide regions derived from another virus or from a cellular source, and recombinantly produced or chemically synthesized polynucleotides which function in a manner similar to the reference nucleotide region to achieve a desired result. Thus, a functional homolog of an adenoviral VA RNA gene region or an adenoviral E2A gene region encompasses derivatives and analogues of such gene regions-including any single or multiple nucleotide base additions, substitutions and/or deletions occurring within the regions, so long as the homologue retains the ability to provide its inherent accessory function to support AAV virion production at levels detectable above background.

The terms “treat”, “treating” and similar terms are used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. A “prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

The terms “subject’, “patient” and “individual” are used interchangeably herein, and refers to a vertebrate to whom an agent is administered, e.g., for experimental, diagnostic, and/or therapeutic purposes or from whom a sample is obtained or on whom a procedure is performed. In some cases, the methods of the invention find use in experimental animals, in veterinary application, and in the development of animal models for disease, including, but not limited to, rodents including mice, rats, and hamsters, and primates. In some embodiments the subject is a mammal, e.g., a human, non-human primate, or rodent (e.g., mouse, rat, rabbit).

As used herein, the term “pharmaceutically acceptable” refers to a molecular entity or composition that does not produce an adverse, allergic or other untoward reaction when administered to an animal or a human, as appropriate. The term “pharmaceutically acceptable carrier”, as used herein, includes any and all solvents, solubilizers, fillers, stabilizers, surfactants, binders, absorbents, bases, buffering agents, excipients, lubricants, controlled release vehicles, diluents, emulsifying agents, humectants, lubricants, gels, dispersion media, coatings, antibacterial or antifungal agents, isotonic and absorption delaying agents, and the like, compatible with pharmaceutical administration. The use of such carriers and agents for pharmaceutically active substances is well-known in the art.

### II. Dual Function Expression Vectors

In one aspect, the present application provides a one or more expression vectors that expresses at least two active agents in infected cells, including at least one siRNA/shRNA or miRNA promoting RNAi-mediated gene silencing. In some embodiments, the present application provides a dual functional expression vector. The dual functional vector comprises a nucleic acid suitably configured for expressing at least two active agents: a first active agent and a second active agent. The first active agent is a siRNA or shRNA or miRNA for silencing endogenous mRNA transcripts from an endogenous gene. The second active agent is the translation product of an allele of the endogenous gene being silenced, where the translation product is expressed from an mRNA transcript that is insensitive to the silencing activity of the first active agent.

In some embodiments, the present application provides at least one dual function expression vector includes a nucleic acid suitably configured for expressing at least two active agents. The first active agent (also referred to as the “first bullet” in the application), corresponds to a siRNA/shRNA or miRNA for “silencing” a mutant disease allele by degrading mRNA transcripts expressed from the endogenous alleles (mutant and wild-type) in a patient suffering from a genetic disease. The second active agent is expressed as a polypeptide corresponding to a wild-type allele of the mutant disease allele being silenced, where the portion of the nucleic acid corresponding to the second active agent, i.e., second active agent is genetically engineered to produce an mRNA transcript that is insensitive to the silencing activity of the first active agent, i.e., a “bullet 1-proof” wide-type allele. The two active agents may be co-expressed from a single expression vector or they may be separately expressed from at least two different expression vectors.

The first step for siRNA/shRNA- or miRNA-mediated gene silencing is the design of a siRNA/shRNA or miRNA sequence that is potent and specific to the intended mRNA to minimize any off-target effect. Methods for designing siRNA/shRNA and miRNA sequences for RNAi are well known in the art.

A conventional siRNA consists of 19-21 nucleotides with two nucleotide overhangs at the 3′ end, usually TT and UU, which are important for recognition by the RNAi machinery. Increasing the length of the dsRNA may enhance its potency, as demonstrated by an in vitro study that dsRNAs with 27 nucleotides were up to 100 times more potent than the conventional siRNAs with 21 nucleotides. The long dsRNAs require processing by Dicer into the shorter siRNAs (hence they are termed as “Dicer-ready” or “Dicer-substrate” siRNAs), which are more efficiently loaded into the RISC, thus facilitating the subsequent gene silencing mechanism. On the other hand, dsRNAs longer than 30 nucleotides can activate the IFN pathway and should be avoided for therapeutic applications. Small hairpin RNAs (shRNA) are sequences of RNA, typically about 45-80 base pairs in length, that include a region of internal hybridization that creates a hairpin structure. shRNA molecules are processed within the cell to form siRNA which in turn knock down gene expression. The benefit of shRNA is that they can be incorporated into plasmid vectors and integrated into genomic DNA for longer-term or stable expression, and thus longer knockdown of the target mRNA.

MicroRNAs (miRNAs) are a class of small noncoding RNAs of ˜22 nt in length which are involved in the regulation of gene expression at the posttranscriptional level by degrading their target mRNAs and/or inhibiting their translation. The goal of using synthetic miRNAs (or miRNA mimics) is to achieve the same biological functions as the endogenous miRNAs. Therefore the synthetic miRNAs should possess the ability to be loaded to RISC and silence the target mRNAs through the natural miRNA signaling pathway. In theory, a single-stranded RNA molecule containing the sequence that is identical to the guide strand of the mature miRNA could be functioned as miRNA mimic. However, the double stranded miRNA containing both guide and passenger strands was found to be 100 to 1,000 times more potent than the single stranded one. The double stranded structure can facilitate the proper loading of the RNA molecule into the RISC, thereby enhancing the gene silencing effect. Therefore, the design of synthetic miRNAs in accordance with the present application relies on the use of miRNA mimics with a duplex structure. Similarly to shRNAs, viral vectors can be used to express miRNAs inside the cells.

In the present application, the viral vector encodes an siRNA/shRNA or miRNA containing an effector sequence of at least 17-80 contiguous nucleotides, which is substantially complementary to a region of the targeted mutant RNA transcript to facilitate silencing of the mutant RNA target. To confer the knock-down efficacy for all the disease-causing mutations of a gene, the silencer bullets usually are designed to cover all mutations by targeting the non-mutant exon region, 5′ primer upstream or 3′ downstream regions.

The nucleic acid encoding the second active agent, a wild type target gene polypeptide, is transcribed to form an mRNA transcript which is resistant to the first active knock-down agent in the dual function recombinant virus. In particular, the coding sequence for the wild type allele in the second active agent is modified by alternative codon usage. A plurality of nucleotides corresponding to the “wobble” position of the triplet code are altered so as to produce a plurality of “silent mutations” in the coding region of the wild-type allele that preserve the original amino acid sequence. As a result, the modified coding sequence for the wild type allele is resistant to RNAi mediated knockdown by the first active agent..

In some embodiments, the effector sequence in the second active agent is less than 85%, less than 84%, less than 83%, less than 82%, less than 81%, less than 80%, less than 79%, less than 78%, less than 77%, less than 76%, less than 75%, less than 74%, less than 73%, less than 72%, less than 71%, less than 70%, or less than 65% identical to the corresponding sequence in the wild-type allele, provided that the amino acid sequence is the same as the amino acid sequence in the corresponding wild-type allele. In some embodiments, the lower limit of identity is at least 60% identical, at least 65% identical or has a percent identity corresponding to one of the percentage values in this paragraph.

In some embodiments, the first active agent encodes an RNAi agent targeting an autosomal dominant gene.

In more particular embodiments, the first active agent encodes a siRNA or miRNA targeting an autosomal dominant or X-linked dominant gene listed in Table 1, Table 2 or Table 3 and the second active agent encodes a polypeptide corresponding to the targeted autosomal or X-linked dominant gene with a wild type amino acid sequence.

In certain instances, a subject with a genetic disease may be found to possess multiple independent mutations in a particular target gene. Accordingly, in some embodiments, the one or more expression vectors of the present application encode a plurality of siRNA/shRNA/miRNA expression units, each targeting a different mutation in the target gene. In these embodiments, the plurality of siRNA/miRNA expression units may be encoded on the same or different expression vectors, alone or in combination with a genetically modified nucleic acid encoding an RNAi resistant wild type allele corresponding to the target gene for therapy.

In other instances, a subject with a genetic disease may be found to possess mutations (single or multiple) in more than one target gene. Accordingly, in some embodiments, the one or more expression vectors of the present application may each encoding one or more siRNA/miRNA expression units targeting a different mutation in one or more target genes alone or in combination with one or more genetically modified nucleic acids encoding RNAi resistant wild type allele expression units corresponding to each target gene for therapy. In these embodiments, the one or more siRNAs/miRNAs may be encoded on the same or different expression vectors. Further, the one or more genetically modified nucleic acids encoding the “bullet-proof” wild type alleles may be encoded on the same or different expression vectors, alone or in combination with one or more siRNA/miRNA expression units.

In some embodiments, the one or more expression vectors may further encode one or more additional active agents providing additive or synergistic effects during treatment. Alternatively, a therapeutic composition comprising the one or more recombinant viruses may be administered in combination with one or more small molecule drugs indicated for the particular disease to be treated.

In certain embodiments, the one or more expression vectors are engineered to direct expression of the first and second active agents ubiquitously (constitutively), preferentially, or specifically in a particular cell type (e.g., where tissue-specific regulatory elements are used to express the polynucleotide). Thus, in certain embodiments, expression of the first and second active agents is under the control of a tissue specific or ubiquitous promoter, such as the CMV promoter or a CMV-chicken beta-actin hybrid (CAG) promoter.

In other embodiments, a tissue-selective, tissue-specific or tumor-specific promoter may be used. Exemplary tissue-selective or tissue-specific regulatory elements are known in the art and may include liver-specific promoters (e.g., albumin promoter), lymphoid-specific promoters, epithelial cell-specific promoters, promoters of T cell receptors and immunoglobulins, neuron-specific promoters (e.g., the neurofilament promoter), retina-specific promoters, pancreas-specific promoters (e.g., insulin promoter), and mammary gland-specific promoters (e.g., milk whey promoter). In other embodiments, developmentally-regulated promoters (e.g., the a-fetoprotein promoter) may be utilized.

In certain particular embodiments, expression of the first and/or second active agents is under the control of promoter/enhancer sequences facilitating expression in one or more cells of the central nervous system (CNS) and/or peripheral nervous system (PNS). In some embodiments, the one or more cells of the CNS comprise one or more cells of the brain. In some embodiments, the one or more cells of the CNS include an oligodendrocyte, astrocyte, neuron, brain parenchyma cell, glial cell, microglial cell, ependymal cell, and/or a Purkinje cell. In some embodiments, the one or more cells include a neuron. The one or more cells of the PNS comprise nerves and ganglia outside the brain and spinal cord.

Exemplary promoters for expression of the first and/or second active agents include, but are not limited to, the cytomegalovirus (CMV) immediate early promoter, an RSV LTR, a MoMLV LTR, a phosphoglycerate kinase-1 (PGK) promoter, a simian virus 40 (SV40) promoter, a CK6 promoter, a transthyretin promoter (TTR), a TK promoter, a tetracycline responsive promoter (TRE), an HBV promoter, an hAAT promoter, a neuron-selective promoter, such as the human synapsin promoter, a muscle-specific promoter, such as the human creatine kinase (MCK) promoter, a liver-specific promoter, such as the human phosphoenolpyruvate carboxykinase (PEPCK) promoter, a rhodopsin kinase promoter, an opsin promoter, a U6 promoter, an E2F promoter, a telomerase (hTERT) promoter, an H1 promoter, a cytomegalovirus enhancer/chicken beta-actin/rabbit β-globin promoter (CAG) promoter, an elongation factor 1-alpha promoter (EF1-α) promoter, a human β-glucuronidase promoter, a chicken 3-actin (CBA) promoter, a retroviral Rous sarcoma virus (RSV) LTR promoter, a dihydrofolate reductase promoter, and a 13-actin promoter.

Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, kidney promoters, endothelial cell promoters, lung tissue promoters, ocular cell promoters, and nervous system promoters which can be used to restrict expression to neurons, astrocytes, or oligodendrocytes.

In some embodiments, one or more promoters are selected for expression of heterologous nucleic acids in a cell of the CNS to treat a disorder or disease of the CNS or PNS. In some embodiments, the promoter drives expression of the active agents in a brain cell. A brain cell may refer to any brain cell known in the art, including without limitation a neuron (such as a sensory neuron, motor neuron, interneuron, dopaminergic neuron, medium spiny neuron, cholinergic neuron, GABAergic neuron, pyramidal neuron, etc.), a glial cell (such as microglia, macroglia, astrocytes, oligodendrocytes, ependymal cells, radial glia, etc.), a brain parenchyma cell, microglial cell, ependemal cell, and/or a Purkinje cell. In some embodiments, the neuron is a medium spiny neuron of the caudate nucleus, a medium spiny neuron of the putamen, a neuron of the cortex layer IV and/or a neuron of the cortex layer V.

CNS-selective or CNS-specific regulatory elements for CNS cells, brain cells, neurons, and glial cells are known in the art and include promoter-regulatory elements corresponding to neuron-specific enolase (NSE), myelin basic protein (MBP), glial fibrillary acid protein (GFAP), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-selective or tissue-specific expression elements for astrocytes include glial fibrillary acidic protein (GFAP) and EAAT2 promoters. A non-limiting example of a tissue-specific expression element for oligodendrocytes is the myelin basic protein (MBP) promoter.

In some embodiments, one or more promoters are selected for driving expression of the active agents in a muscle cell to treat a disorder or disease affecting muscle cells. Non-limiting examples of muscle-selective or muscle-specific promoters include mammalian muscle creatine kinase (MCK) promoters, mammalian desmin (DES) promoters, mammalian troponin I (TNNI2) promoters, and mammalian skeletal alpha-actin (ASKA) promoters (see, e.g., U.S. Patent Publication US 20110212529, the contents of which are herein incorporated by reference in their entirety).

Non-limiting examples of tissue-selective or tissue-specific expression elements for expression in kidney cells include the use of promoters corresponding to Sglt2, Cdh16, nephrin, and kidney-specific cadherin.

In some embodiments, one or more promoters are selected for driving expression of the active agents in an ocular cell type for treating an eye disorder affecting retina cells, retina bipolar cells, photoreceptor cells, rod cells, cone cells, ganglion cells, retinal pigment epithelium (RPE) cells, choroid cells and/or corneal epithelium cells. Exemplary promoters may include retina-specific promoters (e.g., RPE-specific, photoreceptor-specific), cone-specific and/or rod-specific promoters, choroid-specific promoters and cornea-specific promoters. Non-limiting examples of ocular cell-selective or ocular cell-specific promoters include the human G-protein-coupled receptor protein kinase 1 a/k/a rhodopsin kinase 1 (GRK1) promoter, the human interphotoreceptor retinoid-binding protein (hIRBP) proximal promoter, the RPGR proximal promoter, the red opsin promoter, the red-green opsin promoter, the blue opsin promoter, the mouse opsin promoter, the rhodopsin (Rho) promoter, the cone transducin alpha-subunit promoter, the beta phosphodiesterase (PDE) promoter, the retinitis pigmentosa (RP1) promoter, the NXNL2/NXNL1 promoter, the RPE65 promoter, the retinal degeneration slow/peripherin 2 (Rds/perphZ) promoter, the vitelliform macular dystrophy 2 promoter, the IRBP/GNAT2 promoter (hIRBP enhancer fused to cone transducin alpha promoter), the Rds (retinal degeneration slow) promoter, the hPDE6b promoter, and the VEcad promoter (VE-cadherin/Cadherin 5 (CDH5)/CD144 promoter).

In some embodiments, a nucleic acid encoding an active agent may include operably linked natural or heterologous signal peptide domain for secretion of the active agent from cells. The signal peptide sequence is removed from the mature peptide as the mature peptide is secreted from the cell. Since a given signal peptide sequence can affect the level of peptide expression, a peptide-encoded polynucleotide may include any one of a variety of different N-terminal signal peptide sequences known in the art.

In certain embodiments, the one or more expression vectors are non-viral vectors. In some embodiments, the non-viral vectors are plasmids.

In certain embodiments, the one or more expression vectors are viral vectors.

Viral vectors for expression of the first and second active agents may be derived from, e.g., adenoviruses, adeno-associated viruses (AAV), retroviruses (including lentiviruses, such as HIV-1 and HIV-2), vaccinia viruses and other poxviruses, herpesviruses (e.g., herpes simplex virus Types 1 and 2), polioviruses, Sindbis and other RNA viruses, alphaviruses, astroviruses, coronaviruses, orthomyxoviruses, papovaviruses, paramyxoviruses, parvoviruses, picornaviruses, togaviruses and others. The viral vectors may or may not contain sufficient viral genetic information and/or structural components for production of infectious virus when introduced into host cells, i.e., viral vectors may be replication-competent or replication-defective. In some embodiments, e.g., where structural components for production of infectious virus are lacking, the necessary functional components may be supplied in trans by a host cell or by another vector introduced into the cell when production of recombinant virus is desired. Generally speaking, replication-defective recombinant viruses are administered for treatment. A nucleic acid for delivery may be incorporated into a naturally occurring or modified viral genome (or a portion thereof) or may be present within a viral capsid as a separate nucleic acid molecule.

In certain cases, the viral vectors may be engineered to target certain diseases or cell populations by using the targeting characteristics inherent to the virus vector or engineered into the virus vector. Specific cells may be “targeted” for delivery and expression of polynucleotides. Thus, “targeting”, in this case, relates to the use of endogenous or heterologous binding agents in the form of capsids, envelope proteins, antibodies for delivery to specific cells, the use of tissue-specific regulatory elements for restricting expression to specific subset(s) of cells, or both.

In some embodiments, the viral vectors are AAV vectors. AAV vectors provide a preferred delivery system for the nucleic acid therapeutics of the present application, since they can allow for long lasting and continuous expression of functional alleles and silencing of the corresponding mutant alleles. AAV vectors can include or can be modified to control expression of the first and second active agents under a number of different regulatory elements, including various promoter and/or enhancer elements for constitutive or cell-type specific expression.

In certain preferred embodiments, the viral vector is a recombinant adenovirus-associated virus (AAV). AAVs are small (20-26 nm) replication-defective, nonenveloped viruses, that depend on the presence of a second virus, such as adenovirus or herpes virus or suitable helper functions, for replication in cells. AAVs are not known to cause disease and induces a very mild immune response. AAVs can infect both dividing and non-dividing cells and may incorporate its genome into that of the host cell. More than 30 naturally occurring serotypes of AAV are available. Many natural variants in the AAV capsid exist, allowing identification and use of AAV vectors with properties specifically suited for the cell targets of delivery. AAV vectors are relatively non-toxic, provide efficient gene transfer, and can be easily optimized for specific purposes. AAV viruses may be engineered using conventional molecular biology techniques to optimize the generation of recombinant AAV particles for cell specific delivery of the transgenes of the present application, for minimizing immunogenicity, enhancing stability, delivery to the nucleus, etc.

Any suitable AAV serotype or AAV pseudotypes may be used to express the transgenes of the present application in cells in vitro and in vivo. The types of vectors for in vivo delivery are preferably chosen based on lack of pre-existing immunity in the host to a selected AAV subtype and stable expression in vivo. Typically, AAV vectors are derived from single-stranded (ss) DNA parvoviruses that are nonpathogenic for mammals. Among the serotypes of AAVs isolated from human or non-human primates, human serotype 2 is the first and best characterized AAV that was developed as a gene transfer vector. Other useful AAV serotypes include AAV1, AAV3, AAV4, AAV5, AAV6, AAV7, AAV8, AAV9, AAV9.47, AAV9(hu14), AAV10, AAV11, AAV12, AAVrh8, AAVrh10, AAV-DJ8 and AAV-DJ.

In some embodiments, the AAV vector may be a pseudotyped AAV vector containing sequences and/or components originating from at least two different AAV serotypes. Thus, a pseudotyped (or chimeric) AAV vector may include, for example, an AAV2-derived genome in an AAV1-derived or AAV6-derived capsid; or an AAV2-derived genome in an AAV4-derived capsid; or an AAV2-derived genome in an AAV9-derived capsid. Alternatively, a pseudotyped AAV vector may include a portion of the capsid from one AAV serotype fused to a second portion of a different AAV serotype capsid, resulting in a vector encoding a pseudotyped AAV2/AAV5 capsid. In other embodiments, a pseudotyped AAV vector may include a capsid from one serotype and inverted terminal repeats (ITRs) from another AAV serotype. Exemplary AAV vectors include recombinant pseudotyped AAV2/1, AAV2/2, AAV2/5, AAV2/7, AAV2/8 and AAV2/9 serotype vectors.

Generally, recombinant AAV-based vectors are replication defective, because they have the rep and cap (capsid) viral genes removed (which account for 96% of the viral genome), leaving the two flanking 145-basepair (bp) inverted terminal repeats (ITRs), which are used to initiate viral DNA replication, packaging and integration. Typically, an AAV vector can accommodate a “minigene” of about 4.5 kb in length comprising one or more transgenes, each operably linked to one or more regulatory elements for expression.

Unless otherwise specified, the AAV ITRs and other selected AAV components described herein, may be readily selected from among any of the aforementioned serotypes or other known or as yet unknown AAV serotypes. These ITRs or other AAV components may be readily isolated from an AAV serotype using standard techniques known to those of ordinary skill in the art. In addition, AAV sequences may be isolated or obtained from academic, commercial, or public sources (e.g., the American Type Culture Collection, Manassas, Va.) or may be obtained through synthetic or other suitable means by reference to published sequences such as are available in the literature or in databases such as, e.g., GenBank, PubMed and the like.

In some embodiments, the AAV vector is a self-complementary AAV vector (scAAV). Whereas a standard AAV vector has 4.6 Kb of single stranded DNA, an scAAV vector has 2.3 Kb of double stranded DNA. Thus, scAAV vectors contain two DNA strands which anneal together to form double stranded DNA. By skipping second strand synthesis, scAAVs allow for rapid expression in the cell. scAAV vectors have additional advantages over single stranded vectors in terms of their ability to effectively transduce cells. scAAV vectors pseudotyped with AAV8 capsid proteins have been shown to transduce upwards of 95% of targeted liver cells (see e.g., Nakai et al J Virol. 2005 January; 79(1):214-24 and Grimm et al, J Virol. 2006 January; 80(1):426-39).

An AAV vector can be converted into a self-complementary vector by introducing a mutation/deletion in one of the inverted terminal repeats (ITR). Each AAV genome has two such repeats at the 5′ and 3′ ends. Replication typically starts at one of the ITRs and commences through the genome and resolves at the other ITR. It is for this reason that AAV vectors contain genomes that are either positive or negative stranded. The sequences that govern transcriptional resolution most definitively are the D-sequence and the terminal resolution site (trs). These sequences are contained between nucleotides 122-144 of the AAV2 genome (Wang et al (2003) Gene Therapy 10: 2106-2111); deletion of these sequences prevents transcriptional resolution at an ITR. It should be noted that, since the ITRs of AAV vectors are nearly identical, deletion of the D-sequence and trs can be done in either of the two ITRs. As a result of the ITR D-sequence and trs deletion(s), an elongating replication complex can no longer resolve, and the complex continues in an orientation opposite to the original direction. Thus, if the replication complex first generates a positive strand, it fails to resolve at the deleted ITR and instead generates a negative strand that is complementary to the positive strand. This results in a self-complementary double stranded DNA molecule that will get packaged in the AAV vector provided its length is not over 2.3 kb and is preferably shorter.

Since ITRs of an AAV vector recombine during the replication process, a revertant phenotype may result in which both ITRs regain wild type sequences. In order to alleviate this problem, ITRs of different AAV vectors can be used, such as e.g., an AAV2 left ITR with an AAV4 deleted right ITR, etc. The sole criterion governing the choice of ITRs to be combined lies in the sequence identity between the ITRs of the serotype. The ITRs of serotypes 2 and 5 are nearly identical, and the ITRs of serotypes 2 and 4 have an 81.6% similarity. After deletion of the D sequence and trs, the sequence identify between the ITRs of AAV 2 and AAV 4 drops to just over 50%. The combination of these two ITRs therefore generates sufficiently divergent ITRs and will result in an scAAV vector that can no longer regenerate progeny with wildtype ITRs.

Exemplary AAV fragments for assembly into vectors and/or helper cells include the capsid subunit proteins, vp1, vp2, vp3, hypervariable regions, and the rep proteins, rep 78, rep 68, rep 52, and rep 40. When providing the AAV rep and cap products, the AAV rep and AAV cap sequences can both be of one serotype origin, e.g., all AAV8 origin or may be derived from multiple AAV serotypes. In one embodiment, the rep and cap sequences are expressed from separate sources (e.g., separate vectors, or a host cell and a vector). In some embodiments, these rep sequences may be fused in frame to cap sequences of a different AAV serotype to form a chimeric AAV vector, such as AAV2/8 described in U.S. Pat. No. 7,282,199.

A recombinant adeno-associated virus (rAAV) may be generated by culturing a host cell which contains a nucleic acid sequence encoding an adeno-associated virus (AAV) serotype capsid protein, or fragment thereof, as defined herein; a functional rep gene; a vector composed of, at a minimum, AAV inverted terminal repeats (ITRs) and the fusion protein encoding nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. For example, a stable host cell may be generated which is derived from 293 cells (which contain E1 helper functions under the control of a constitutive promoter), but which contains the rep and/or cap proteins under the control of constitutive or inducible promoters.

The minigene, rep sequences, cap sequences, and helper functions required for producing a recombinant AAV (rAAV) of the present disclosure may be delivered or contained in a packaging host cell or helper cell. The selected genetic element may be delivered using any suitable method, including those described herein and any others available in the art. Non-limiting methods of generating rAAV virions are well known in the art.

In certain embodiments, rAAVs can spread throughout CNS tissue following direct administration into the cerebrospinal fluid (CSF), e.g., via intrathecal and/or intracerebral injection. In some embodiments, rAAVs (such as AAV-9 and AAV-10) cross the blood-brain-barrier and achieve wide-spread distribution throughout CNS tissue of a subject following intravenous administration. In some cases, intravascular (e.g., intravenous) administration facilitates the use of larger volumes than other forms of administration (e.g., intrathecal, intracerebral). Thus, large doses of rAAVs (e.g., up to 10¹⁵ rAAV genome copies/subject) can be delivered at one time by intravascular (e.g., intravenous) administration. Methods for intravascular administration are well known in the art and include, for example, use of a hypodermic needle, peripheral cannula, central venous line, etc.

In another aspect, a genetic disease treatment system includes CRISPR knockout for the first active agent (Bullet 1) and the second active agent encodes a wild-type polypeptide corresponding to the target gene (Bullet 2). For Bullet 1, plasmid or viral vectors encoding a gene editing system comprised of (1) a guide RNA (gRNA) targeting the site of gene editing; and (2) a nuclease comprising a Clustered Regularly Interspaced Short Palindromic Repeats (CRISPR)-associated (Cas) protein, such as Cas9, a zinc finger nuclease (ZFN), a Transcription Activator-Like Effector Nuclease (TALEN), or a meganuclease. With Cas9 nuclease complexed and a synthetic gRNA, the cell's genome can be cut at a desired location. The cleavage is repaired by non-homologous end joining (NHEJ). NHEJ can often result in random deletions or insertions at the repair site, which result in target gene (includes mutant and wild-type alleles) knockout. Bullet 2 supplements the expression of wild-type allele and resistant to Bullet 1 knockout by alternative codon usage.

In addition to the naturally occurring guide RNAs, synthetic guide RNAs can be fused to a CRISPR vector. The design of guide RNAs with target-recognition sequences and other essential elements (e.g., hairpin and scaffold sequence) using bioinformatics methods is described (see, e.g., Mali et al., Science 339: 823-826 (2013)).

### III. Diseases for Treatment

A wide variety of diseases are contemplated for treatment in accordance with the present application including, but are not limited to, neurological diseases, inflammatory and immune diseases, muscular and bone disorders, cardiac and vascular disorders, retinal and ophthalmology diseases, developmental diseases, metabolic diseases, and various cancers or lymphoproliferative disorders.

In some embodiments, the disease is caused by one or more autosomal dominant mutations or X-linked dominant mutations in a single gene. Non-limiting examples of diseases caused by autosomal dominant or X-linked dominant mutations to be targeted by the first active agent, along with the corresponding wild-type protein products expressed by the second active agent are shown in Table 1 and Table 3.

In some embodiments, the disease is caused by one or more X-linked recessive mutations in a single gene. Non-limiting examples of diseases caused by X-linked recessive mutations to be targeted by the first active agent, along with the corresponding wild-type protein products expressed by the second active agent are shown in Table 2 and Table 3.

1. Neurological Diseases

In one embodiment, the disease for treatment is a neurological disease or disorder. Neurological diseases or disorders for treatment in accordance with the present application include, but are not limited to, amyotrophic lateral sclerosis (ALS), including familial ALS (fALS), peripheral small-fiber neuropathy, various spinocerebellar ataxias, Huntington's disease, Parkinson's disease, Alzheimer's disease, and other neurological diseases or disorders listed in Tables 1-3.

1.1. Familial Amylotrophic Lateral Sclerosis (fALS): SOD1/C9ORF72

Amyotrophic lateral sclerosis (ALS), also known as Lou Gehrig's disease, is a devastating, untreatable neurodegenerative disease, characterized by the predominant loss of motor neurons (MNs) in primary motor cortex, the brainstem, and the spinal cord. The loss of motor neurons devastates basic fundamental movements, such as breathing, and typically causes death to patients within about 2-4 years after diagnosis. Onset of the disease typically occurs between the ages of 40-70, although an early onset form of the disease has been found in patients between the ages of 20-30. Progressive deterioration of motor neuron function in patients severely disrupts their breathing ability, requiring some form of breathing aid for survival. Other symptoms also include spasticity, cramps, fasciculation, and muscle weakness in hands, arms, legs or the muscles of swallowing. Some patients (e.g., FTD-ALS) may also develop frontotemporal dementia.

According to the ALS Association, approximately 5,600 people in the United States of America are diagnosed with ALS each year. The incidence of ALS is two per 100,000 people, and it is estimated that as many as 30,000 Americans may have the disease at any given time.

Two clinically indistinguishable forms of ALS have been described: (1) sporadic ALS (sALS), which is the most common form of ALS in the United States of America and accounts for about 90% of all diagnosed cases; and (2) familial ALS (fALS), which occurs in a patients with a family history of disease reflected in an autosomal dominant inheritance pattern that accounts for about 10% of all diagnosed cases.

ALS is generally considered to be a complex genetic disorder in which multiple genes in combination with environmental exposures can combine to render a person susceptible. More than a dozen mutated genes associated with ALS have been discovered, including, but not limited to, those affecting: (1) C9ORF72 (chromosome 9 open reading frame 72), which account for about 40% of fALS cases, about 7% of sALS cases, and about 10% of all ALS cases; (2) SOD1 (Cu²⁺/Zn²⁺ superoxide dismutase type I), which account for about 1²-20% of fALS cases, about 2% of sALS cases, and about 4% of all ALS cases; (3) TDP-43 (TAR DNA binding protein of 43 kDa), which account for about 5% of fALS cases, about 1% of sALS cases, and about 0.6% of all ALS cases; and (4) FUS (fused in sarcoma), which account for about 4% of fALS cases, about 1% of sALS cases, and about 0.5% of all ALS cases. Additional mutated genes include ANG (Angiogenin), ATXN2 (Ataxin-2), VCP (valosin containing protein), and OPTN (Optineurin).

C9ORF72-linked fALS is associated with both gain of function and loss of function activities. The mutations in C9ORF72 typically include a hexanucleotide repeat expansion (GGGGCC)ₙ representing the most frequent cause of fALS in Western populations. The hexanucleotide repeat expansion may range from about 20-30 repeats to hundreds of repeats in mutated C9ORF72.

SOD1-linked fALS is most likely not caused by loss of the normal SOD1 activity, but rather by a gain of a toxic function, reflected in axonal transport defects, mitochondrial dysfunction, increased ER stress, increased glutamate toxicity, and/or caspase activation. Currently, at least 170 different mutations distributed throughout the 153-amino acid SOD1 polypeptide have been found to cause ALS, and an updated list can be found at the ALS online Genetic Database (ALSOD) (Wroe R et al., Amyotroph Lateral Scler., 2008, 9, 249-250).

Table 4 lists examples of mutations in SOD1 in ALS. These mutations are predominantly single amino acid substitutions (i.e. missense mutations) although deletions, insertions, and C-terminal truncations also occur. Different SOD1 mutations display different geographic distribution patterns. For instance, about half of all Americans with ALS caused by SOD1 gene mutations have a particular mutation Ala4Val (or A4V). The A4V mutation is typically associated with more severe signs and symptoms. The 1113T mutation is by far the most common mutation in the United Kingdom. The most prevalent mutation in Europe is D90A substitute.

TABLE 4
SOD1 mutations in ALS.

| Exon | Base | |
|---|---|---|
| No. | pairs | Mutations |
| 1 | 220 | Q22L; E21K, G; F20C; N19S; G16A, S; V14M, S; G12R; G10G, V, R; |
| | | L8Q, V; V7E; C6G, F; V5L; A4T, V, S |
| 2 | 97 | T54R; E49K; H48R, Q; V47F, A; H46R; F45C; H43R; G41S, D; G37R; |
| | | V29, insA |
| 3 | 70 | D76Y, V; G72S, C; L67R; P66A; N65S; S59I, S |
| 4 | 118 | D124G, V; V118L, InsAAAAC; L117V; T116T; R115G; G114A; |
| | | I113T, F; I112M, T; G108V; L106V, F; S106L, delTCACTC; I104F; |
| | | D101G, Y, H, N; E100G, K; I99V; V97L, M; D96N, V; A95T, V; |
| | | G93S, V, A, C, R, D; D90V, A; A89T, V; T88delACTGCTGAC; V87A, M; |
| | | N86I, S, D, K; G85R, S; L84V, F; H80R |
| 5 | 461 | I151T, S; I149T; V148I, G; G147D, R; C146R, stop; A145T, G; L144F, S; |
| | | G141E, stop; A140A, G; N139D, K, H, N; G138E; T137R; S134N; |
| | | E133V, delGAA, insTT; E132insTT; G127R, InsTGGG; |
| | | L126S, delITT, stop; D126, delTT |

One of the hypotheses for mutant SOD1-linked fALS toxicity proposes that an aberrant SOD1 enzyme causes small molecules such as peroxynitrite or hydrogen peroxide to produce damaging free radicals. Other hypotheses for mutant SOD1 neurotoxicity include inhibition of the proteasome activity, mitochondrial damage, disruption of RNA processing and formation of intracellular aggregates. Abnormal accumulation of mutant SOD1 variants and/or wild-type SOD1 in ALS forms insoluble fibrillar aggregates which are identified as pathological inclusions. Aggregated SOD1 protein can induce mitochondria stress and other toxicity to cells, particularly to motor neurons.

1.2. Huntington's Disease (HD): Huntingtin Protein (HTT)

Huntington's disease (HD) is caused by a CAG repeat expansion mutation that encodes an expanded polyglutamine (polyQ) repeat in the Huntingtin protein (HTT), resulting in a mutant Huntingtin protein (mHTT). Symptoms of HD include, but are not limited to, chorea, rigidity, uncontrollable body movements, loss of muscle control, lack of coordination, restlessness, slowed eye movements, abnormal posturing, instability, ataxic gait, abnormal facial expression, speech problems, difficulties chewing and/or swallowing, disturbance of sleep, seizures, dementia, cognitive deficits (e.g., diminished abilities related to planning, abstract thought, flexibility, rule acquisition, interpersonal sensitivity, self-control, attention, learning, and memory), depression, anxiety, changes in personality, aggression, compulsive behavior, obsessive-compulsive behavior, hypersexuality, psychosis, apathy, irritability, suicidal thoughts, weight loss, muscle atrophy, heart failure, reduced glucose tolerance, testicular atrophy, and osteoporosis.

Autosomal dominant HD can be genotyped to determine the number of CAG repeats by PCR-based repeat sizing. This type of diagnosis may be performed at any stage of life through directly testing juveniles or adults (e.g., along with presentation of clinical symptoms), prenatal screening or prenatal exclusion testing (e.g., by chorionic villus sampling or amniocentesis), or preimplantation screening of embryos. Additionally, HD may be diagnosed by brain imaging, looking for shrinkage of the caudate nuclei and/or putamen and/or enlarged ventricles. These symptoms, combined with a family history of HD and/or clinical symptoms, may indicate HD.

Means for determining amelioration of the symptoms of HD are known in the art. For example, the Unified Huntington's Disease Rating Scale (UHDRS) may be used to assess motor function, cognitive function, behavioral abnormalities, and functional capacity (see, e.g., Huntington Study Group (1996) Movement Disorders 11:136-42). This rating scale was developed to provide a uniform, comprehensive test for multiple facets of the disease pathology, incorporating elements from tests such as the HD Activities and Daily Living Scale, Marsden and Quinn's chorea severity scale, the Physical Disability and Independence scales, the HD motor rating scale (HDMRS), the HD functional capacity scale (HDFCS), and the quantitated neurological exam (QNE). Other tests useful for determining amelioration of HD symptoms may include without limitation the Montreal Cognitive Assessment, brain imaging (e.g., MRI), Category Fluency Test, Trail Making Test, Map Search, Stroop Word Reading Test, Speeded Tapping Task, and the Symbol Digit Modalities Test.

In some aspects of the invention, the methods and compositions are used for the treatment of humans with HD. As described above, HD is inherited in an autosomal dominant manner and caused by CAG repeat expansion in the HTT gene. Juvenile-onset HD is most often inherited from the paternal side. Huntington disease-like phenotypes have also been correlated with other genetic loci, such as HDL1, PRNP, HDL2, HDL3, and HDL4. It is thought that other genetic loci may modify the manifestation of HD symptoms, including mutations in the GRIN2A, GRIN2B, MSX1, GRIK2, and APOE genes.

1.3. Parkinson's Disease (PD): LRRK2

Parkinson's disease (PD) is a neurodegenerative disease that afflicts approximately 4-6 million people worldwide. In the United States, approximately one to two hundred people per 100,000 have PD. The prevalence of PD increases in the older population, with approximately 4% of people over the age of 80 suffering from this disease (Davie (2008) Brit Med Bull 86(1) p. 109), although 10% of patients are under 40 years of age (Kumari (2009) FEBS J. 276(22) p. 6455).

It appears that many factors can play a role in disease onset and/or progression of PD. For example, genetic mutations in the leucine rich repeat kinase 2 gene (LRRK2, also known as PARK8) have been identified in both familial and sporadic forms of PD. In fact, studies suggest that LRRK2 mutations may be responsible for between 5 and 13% of familial PD, and from 1 to 5% of sporadic PD. The protein itself is a large (>280 kD) multidomain protein containing the following known domains: armadillo (ARM), ankryn (ANK), LRR, Ras of complex proteins (ROC), C-terminal of ROC (COR), mitogen-activated protein kinase and WD40. Thus, LRRK2 contains several protein-protein interactive domains (ARM/ANK, LRR and WD40) suggesting that LRRK2 plays a role in protein complex formation (Kumari, ibid). Several clusters of mutations have been identified which fall across its length of the gene, with the majority of pathological mutations clustering in the enzymatic domains of the protein.

Specifically, the LRRK2 mutation G2019S has been suggested to play an important role in PD in some ethnicities. The mutation is autosomal dominant and the lifetime penetrance for the mutation has been estimated at 31.8%. The SNP responsible for this missense mutation in patients is annotated as rs34637584 in the human genome, and is a G to A substitution at the genomic level (6055G>A). This LRRK2 mutation can be referred to either as G2019S or 6055G>A and is found at or near chr12:40734202. The G2019S mutation has been shown to increase LRRK2 kinase activity, and is found within the activation domain or protein kinase-like domain of the protein.

1.4. Mental Retardation-5 (MRD-5): SYNGAP1

Mental retardation is the most prevalent handicap of children affecting 1 to 3% of the population. Autosomal dominant mental retardation-5 (MRD5) is a prevalent nonsyndromic form of the disorder characterized by the lack of associated morphologic, radiologic, and metabolic features. A case study identified de novo genetic lesions in the SYNGAP1 gene that result in the production of truncated proteins in approximately 3% of patients with unexplained nonsyndromic mental retardation. SYNGAP1 is a GTPase-activating enzyme that is selectively expressed in the brain and required for normal development (Hamden, et al., 2009, NEJM 360: 599-605).

2. Inflammatory or Immune Diseases

In another embodiment, the disease for treatment is an inflammatory or immune diseases. Inflammatory and immune diseases for treatment in accordance with the present application include, but are not limited to, hereditary angioedema (HAE), systemic mastocytosis, infantile enterocolitis, etc.

3. Muscular and Bone Disorders

In another embodiment, the disease for treatment is a muscular disorder. Muscular disorders for treatment in accordance with the present application include, but are not limited to, myotonic dystrophies, limb-girdle muscular dystrophies, centronuclear myopathies, congenital myopathies, GNE-related myopathy, and others.

Autosomal dominant type 1 myotonic dystrophy type 1 (DM1) and type 2 myotonic dystrophy (DM2) are both caused by abnormally expanded stretches of DNA corresponding to myotonic dystrophy protein kinase (DMPK), which plays a critical role in turning off (or inhibiting) the muscle protein, myosin phosphatase. Myosin phosphatase is an enzyme that plays a role in muscle tensing (contraction) and relaxation. DM1 affects between 1 in 100,000 people in populations of Japan to 1 in 10,000 people in Iceland. In the United States the incidence of DM1 is estimated to be about 1 in 8,000 people worldwide. Common symptoms of DM1 include muscle weakness and wasting, prolonged muscle tensing (myotonia), cataracts, and arrhythmias. No specific treatment for the muscle weakness associated with DM1 is currently available.

Autosomal dominant centronuclear myopathy (AD-CNM) results from mutations in the DNM2 gene. AD-CNM is a rare congenital myopathy characterized by the high incidence of centrally placed nuclei in muscle fibers in absence of regenerative process. The AD-CNM is associated with a wide clinical spectrum from severe-neonatal to mild-adult forms. In general, motor milestones are delayed and diffuse skeletal muscle weakness mainly involves facial and limb muscles. Muscle weakness is slowly progressive but loss of independent ambulation may occur during the fifth decade. In the severe and early-onset CNM, pediatric patients usually have generalized weakness, hypotonia, moderate degree of facial weakness with open mouth, ptosis and ophthalmoplegia. No curative treatment is available for the AD-CNM. Mutations in the DNM2 gene are also involved in rare cases of Charcot-Marie-Tooth disease and hereditary spastic paraplegia.

Charcot-Marie-Tooth disease (CMT) is a hereditary motor and sensory neuropathy characterized by progressive loss of muscle tissue and touch sensation across various parts of the body. Hereditary spastic paraplegia (HSP) is an inherited disease characterized by lower extremity spasticity and weakness occurring in variable proportion.

4. Cardiac and Vascular Diseases

In another embodiment, the disease for treatment is a cardiac disease. Cardiac diseases for treatment in accordance with the present application include, but are not limited to, inherited channelopathies, familial atrial fibrillation, Timothy syndrome, etc.

5. Retinal and Ophthalmology Diseases

In another embodiment, the disease for treatment is a retinal disease. Retinal diseases for treatment in accordance with the present application include, but are not limited to, autosomal dominant retinitis pigmentosa, late-onset retinal degeneration, etc.

Retinitis pigmentosa (RP) refers to a diverse group of hereditary diseases affecting two million people worldwide that lead to incurable blindness. RP is one of the most common forms of inherited retinal degeneration, and there are multiple autosomal dominant and X-linked recessive genes whose mutation(s) can lead to RP. More than 100 mutations in 44 genes expressed in rod photoreceptors have thus far been identified, accounting for 15% of all types of retinal degeneration, most of which are missense mutations and are usually autosomal dominant.

Rhodopsin is a pigment of the retina that is involved in the first events in the perception of light. It is made of the protein moiety opsin covalently linked to a retinal cofactor. Rhodopsin is encoded by the RHO gene (chr3:129247482-129254187), and the protein has a molecular weight of approximately 40 kD and spans the membrane of the rod cell. The retinal cofactor absorbs light as it enters the retina and becomes photoexcited, causing it to undergo a change in molecular configuration, and dissociates from the opsin. This change initiates the process that eventually causes electrical impulses to be sent to the brain along the optic nerve.

With regard to RP, more than 80 mutations in the rhodopsin gene have been identified that account for 30% of all autosomal dominant retinitis pigmentosa (ADRP) cases in humans. Three point mutations in the human rhodopsin gene (leading to P23H, Q64X and Q344X in the protein sequence) are known to cause ADRP in humans. See, e.g., Olsson et al. (1992) Neuron 9(5):815-30. The P23H mutation is the most common rhodopsin mutation in the United States.

6. Developmental Diseases

In another embodiment, the disease for treatment is a developmental disease. Developmental diseases for treatment in accordance with the present application include, but are not limited to, Hyperinsulinism-hyperammonemia syndrome, etc.

7. Metabolic Diseases

In another embodiment, the disease for treatment is a metabolic disease. Metabolic diseases for treatment in accordance with the present application include, but are not limited to, autosomal dominant hypercholesterolemia, Hunter syndrome (X-linked; AD in males; iduronate-2-sulfatase (IDS)), etc.

8. Cell Proliferative Disorder

In another embodiment, the disease for treatment is a cell proliferative disorder. The term “cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “cell proliferative disorder” is a neoplasm or cancer or tumor, which is an abnormal growth of tissue.

“Cancer or tumor” refers to any one of a variety of malignant neoplasms characterized by the proliferation of cells that have the capability to invade surrounding tissue and/or metastasize to new colonization sites, and includes leukemia, lymphoma, carcinoma, melanoma, sarcoma, germ cell tumor and blastoma. Exemplary cancers for treatment with the methods of the instant disclosure include cancer of the brain, bladder, breast, cervix, colon, head and neck, kidney, lung, non-small cell lung, mesothelioma, ovary, prostate, stomach and uterus, leukemia, and medulloblastoma.

The term “leukemia” refers to progressive, malignant diseases of the blood-forming organs and is generally characterized by a distorted proliferation and development of leukocytes and their precursors in the blood and bone marrow. Exemplary leukemias include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to the malignant growth of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” refers to a tumor made up of a substance like the embryonic connective tissue and is generally composed of closely packed cells embedded in a fibrillar or homogeneous substance. Exemplary sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer.

### IV. Development of Animal Models for Human Diseases

In another aspect, the present application provides a method for developing animal models using the expression vectors of the present application. The animal models can be models of human genetic diseases or genetically humanized animals in which an animal sequence is knock-down (or knock-out) and replaced by the human orthologous DNA. In this case, the dual functional expression vector represents the converse of the dual functional expression vector for genetic disease treatment described above. Specifically, the dual functional expression vector for these embodiments includes a nucleic acid suitably configured for expressing at least two active agents. In contrast to the dual functional expression vector for genetic disease treatment described above, the first active agent is an siRNA or miRNA for silencing an mRNA transcript expressed from a wild type allele corresponding to a mutated allele known to cause an autosomal dominant, X-linked dominant or X-linked recessive genetic disease and the second active agent is a mutant polypeptide corresponding to the mutated allele. Furthermore, the nucleic acid is genetically engineered to produce an mRNA transcript for the mutant polypeptide that is insensitive to the silencing activity of the first active agent

In one embodiment, the animal can be mouse, rat, pig and non-human primates.

In one embodiment, the genetic disease for the animal model is listed in Table 1 and the first and second active agents are directed to the corresponding gene or polypeptide associated therewith.

In another embodiment, the genetic disease for animal model is listed in Table 2 and the first and second active agents are directed to the corresponding gene or polypeptide associated therewith.

In a particular embodiment, the genetic disease for the animal model is hereditary angioedema (HAE), siRNA/shRNA or miRNA is targeted wild type C1 esterase inhibitor transcripts, and the second active agent is a mutant type C1 esterase inhibitor polypeptide that is insensitive to the silencing activity of the first active agent.

In some embodiments, the animal models are humanized animals as important pre-clinical tools for studying and developing treatment of human infectious diseases, which include, but are not limited to hepatitis B virus (HBV) infection, coronavirus disease 2019 (COVID-19), and etc.

Chronic infection with hepatitis B virus (HBV) infection is one of the most common viral infections and the leading cause of liver diseases and cancer in humans worldwide. Mammalian HBV-like viruses are also found in nonhuman primates, rodents, and bats. HBV requires a successful interaction with a host receptor for replication. In human, HBV interacts with the cellular sodium taurocholate cotransporting polypeptide (NTCP/SLC10A1) to infect hepatocytes. Mouse NTCP is HBV-resistant. Replacing HBV-NTCP-binding determinants in mouse with human counterparts rendered mouse NCTP functional receptors for HBV infections.

SARS-CoV-2, the novel coronavirus responsible for COVID-19 utilizes Angiotensin-Converting Enzyme 2 (ACE-2) as the entry receptor to infect host cells. ACE-2 is a type I transmembrane metallocarboxypeptidase with homology to ACE, an enzyme long-known to be a key player in the Renin-Angiotensin system (RAS) and a target for the treatment of hypertension. To study pathogenicity of SARS-CoV-2 virus for prevention and treatment of the disease, it is critical to establish animal models expressing human ACE2.

In some embodiments, the expression vectors are non-viral vectors. In other embodiments, the expression vectors are viral vectors. In some embodiments, the viral vectors are derived from an adenovirus, an adenovirus-associated viruses (AAV), a retrovirus, or a lentivirus. In a particular embodiment, the expression vector is a recombinant AAV virus (rAAV).

In some embodiments, the method includes the steps of: administering an effective amount of the aforementioned recombinant virus in one or more animal species; screening injected animals for proper delivery of virus to the designed tissues; examining the expression level of the endogenous mutant and wild-type protein for efficacy of knockdown by Bullet 1 and the expression of the codon engineered wild type or protein for efficacy of supplement by Bullet 2; and selecting animals for use as a model for the particular genetic disease or humanization targeted by the recombinant virus.

In another aspect, the present application provides an animal as a disease model for a particular disease targeted by the recombinant virus above. In a particular embodiment, the animal disease model pertains to a genetic disease listed in Table 1 in which the first and second active agents are directed to the corresponding genetic disease in accordance with the aforementioned strategy. In another embodiment, the animal disease model pertains to a genetic disease listed in Table 2 in which the first and second active agents are directed to the corresponding genetic disease in accordance with the aforementioned strategy. In more particular embodiments, the animal disease model pertains to fALS or HAE. In another embodiment, the animal model pertains to a genetically humanized animals in which an animal gene is deleted or knock-downed by active reagent 1 and replaced by the human orthologous gene by active reagent 2.

### V. Pharmaceutical Composition for Treatment of a Genetic Disease

Another aspect of the present application relates to a pharmaceutical composition for treatment of a genetic disease described herein. The pharmaceutical composition comprises a dual function expression vector of the present application and a pharmaceutically acceptable carrier. In some embodiments, the dual function expression vector comprises a nucleic acid suitably configured for expressing at least two active agents, including at least one siRNA/shRNA or miRNA for silencing or knockdown of an mRNA transcript expressed from the mutant disease allele in a patient suffering from a genetic disease, and a second active agent in the form of a polypeptide corresponding to a wild-type allele of the mutant disease allele being silenced, wherein the nucleic acid is genetically engineered to produce an mRNA transcript that is insensitive to the activity (and silencing) of the first active agent. The two active agents may be co-expressed from a single expression vector or they may be separately expressed from at least two different viral vectors or viral particles.

In other embodiments, the pharmaceutical composition comprises a plurality of recombinant viruses, each encoding one or more siRNA/shRNA/miRNA expression units targeting a different mutation in one or more target genes alone or in combination with one or more genetically modified nucleic acids encoding RNAi resistant wild type allele expression units corresponding to each target gene for therapy. In these embodiments, the one or more siRNA//shRNA/miRNAs may be encoded on the same or different recombinant viruses. Further, the one or more genetically modified nucleic acids encoding the RNAi resistant wild type alleles may be encoded on the same or different recombinant viruses, alone or in combination with one or more siRNA/miRNA expression units.

In some embodiments, the viral titer of the pharmaceutical composition is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL.

In some embodiments, the viral titer of the pharmaceutical composition is encompassed in a range between 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹² 20×10¹² to 25×10¹², 25×10¹² to 30×10¹², 30×10¹² to 50×10¹², or 50×10¹² to 100×10¹² genome copies/mL. In some embodiments, the viral titer of the pharmaceutical composition is encompassed in a range from about 5×10¹² to 10×10¹², 10×10¹² to 25×10¹², or 25×10¹² to 50×10¹² genome copies/mL.

In some embodiments, the viral titer of the pharmaceutical composition for administration is at least about 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the pharmaceutical composition for administration is encompassed by a range from about 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL.

In some embodiments, the viral titer of the pharmaceutical composition for administration is at least about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some embodiments, the viral titer of the pharmaceutical composition for administration is encompassed by a range from about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some embodiments, the viral titer of the pharmaceutical composition for administration is encompassed by a range from about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL.

In some embodiments, the dose of viral particles administered to the individual in an amount of at least 1×10⁸, at least 1×10⁹, at least a×10¹⁰, at least 1×10¹¹, at least 1×10¹², or at least 1×10¹³ genome copies/kg of body weight with an upper limit including any of the aforementioned doses or up to 1×10¹⁴ or 1×10¹⁵ genome copies/kg of body weight.

In some embodiments, the method of treating a disorder of the nervous system comprises delivery of recombinant viruses to cells in the central nervous system (CNS). In certain embodiments, the recombinant viruses are rAAV particles delivered to the CNS of the individual. In particular embodiments, the administration comprises direct spinal cord injection, intacranial and/or intracerebral administration. In some embodiments, the intracerebral administration is at a site selected from the group consisting of the cerebrum, medulla, pons, cerebellum, intracranial cavity, meninges surrounding the brain, dura mater, arachnoid mater, pia mater, cerebrospinal fluid (CSF) of the subarachnoid space surrounding the brain, deep cerebellar nuclei of the cerebellum, ventricular system of the cerebrum, subarachnoid space, striatum, cortex, septum, thalamus, hypothalamus, and the parenchyma of the brain. In some embodiments, the administration is intracerebroventricular injection into at least one cerebral lateral ventricle. In some embodiments, the administration is intrathecal injection in the cervical, thoracic, and/or lumbar region. In some embodiments, the administration is intrastriatal injection. In some embodiments, the administration is intrathalamic injection. In some embodiments, the administration is intraparenchymal injection. In some embodiments, the rAAV particles are administered at a single site. In other embodiments, the rAAV particles are administered at a plurality of sites.

In some embodiments, the rAAV particle is delivered by stereotactic delivery. In some embodiments, the rAAV particle is delivered by convection enhanced delivery. In some embodiments, the rAAV particle is administered using a CED delivery system. In particular embodiments, the CED delivery system comprises a cannula and/or a pump. In some embodiments, the cannula is a reflux-resistant cannula or a stepped cannula. In some embodiments, the pump is a manual pump. In some embodiments, the pump is an osmotic pump. In other embodiments, the pump is an infusion pump.

In some embodiments, the method of treatment for certain eye diseases (e.g., retinitis pigmentosa, and age-related macular degeneration etc.) includes delivery of recombinant viruses to cells in the retina.

In some embodiments the rAAV viral are administered to more than one location simultaneously or sequentially. In some embodiment, multiple injections of rAAV viral particles are no more than one hour, two hours, three hours, four hours, five hours, six hours, nine hours, twelve hours or 24 hours apart.

Exemplary carriers or excipients for use in the pharmaceutical compositions of the present application include but are not limited to, calcium carbonate, calcium phosphate, various sugars, starches, cellulose derivatives, gelatin, polymers such as polyethylene glycols, water, saline, isotonic aqueous solutions, phosphate buffered saline, dextrose, 0.3% aqueous glycine, glycerol, ethanol and the like, as well as combinations thereof. In many cases, it will be preferable to include isotonic agents, for example, sugars, polyalcohols such as mannitol, sorbitol, or sodium chloride in the composition, or glycoproteins for enhanced stability, such as albumin, lipoprotein and globulin. Pharmaceutically acceptable carriers may further comprise minor amounts of auxiliary substances such as wetting or emulsifying agents, preservatives or buffers, which enhance the shelf life or effectiveness of the therapeutic agents. In certain embodiments, the pharmaceutically acceptable carrier comprises serum albumin.

Formulation characteristics that can be modified include, for example, pH and osmolality. For example, it may be desired to achieve a formulation that has a pH and osmolality similar to that of human blood or tissues to facilitate the formulation's effectiveness when administered parenterally.

Buffers are useful in the present application for, among other purposes, manipulation of the total pH of the pharmaceutical formulation (especially desired for parenteral administration). A variety of buffers known in the art can be used in the present formulations, such as various salts of organic or inorganic acids, bases, or amino acids, and including various forms of citrate, phosphate, tartrate, succinate, adipate, maleate, lactate, acetate, bicarbonate, or carbonate ions. Particularly advantageous buffers for use in parenterally administered forms of the presently disclosed compositions in the present invention include sodium or potassium buffers, including sodium phosphate, potassium phosphate, sodium succinate and sodium citrate.

Sodium chloride can be used to modify the tonicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%).

In one embodiment, sodium phosphate is employed in a concentration approximating 20 mM to achieve a pH of approximately 7.0. A particularly effective sodium phosphate buffering system comprises sodium phosphate monobasic monohydrate and sodium phosphate dibasic heptahydrate. When this combination of monobasic and dibasic sodium phosphate is used, advantageous concentrations of each are about 0.5 to about 1.5 mg/ml monobasic and about 2.0 to about 4.0 mg/ml dibasic, with preferred concentrations of about 0.9 mg/ml monobasic and about 3.4 mg/ml dibasic phosphate. The pH of the formulation changes according to the amount of buffer used.

Depending upon the dosage form and intended route of administration it may alternatively be advantageous to use buffers in different concentrations or to use other additives to adjust the pH of the composition to encompass other ranges. Useful pH ranges for compositions of the present invention include a pH of about 2.0 to a pH of about 12.0.

In some embodiments, it will also be advantageous to employ surfactants in the presently disclosed formulations, where those surfactants will not be disruptive of the drug-delivery system used. Surfactants or anti-adsorbents that prove useful include polyoxyethylenesorbitans, polyoxyethylenesorbitan monolaurate, polysorbate-20, such as Tween-20™, polysorbate-80, polysorbate-20, hydroxycellulose, genapol and BRIJ surfactants. By way of example, when any surfactant is employed in the present invention to produce a parenterally administrable composition, it is advantageous to use it in a concentration of about 0.01 to about 0.5 mg/ml.

Additional useful additives are readily determined by those of skill in the art, according to particular needs or intended uses of the compositions and formulator. One such particularly useful additional substance is sodium chloride, which is useful for adjusting the osmolality of the formulations to achieve the desired resulting osmolality. Particularly preferred osmolalities for parenteral administration of the disclosed compositions are in the range of about 270 to about 330 mOsm/kg. The optimal osmolality for parenterally administered compositions, particularly injectables, is approximately 300 mOsm/kg and achievable by the use of sodium chloride in concentrations of about 6.5 to about 7.5 mg/ml with a sodium chloride concentration of about 7.0 mg/ml being particularly effective. In accordance with the present application, methods for treating the aforementioned genetic disease comprise administering an effective amount of recombinant viral particles encoding the first (RNAi agents) and second active agents (wild type allele).

Pharmaceutical compositions of the present application are formulated to be compatible with its intended route of administration, which is partly determined based on the nature of target cells to be transduced. Examples of routes of administration include parenteral, e.g., intrathecal, intra-arterial, intravenous, intradermal, subcutaneous, transdermal (topical) and transmucosal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the multipartite peptide in the required amount in an appropriate solvent with one or a combination of ingredients enumerated above, as required, followed by filtered sterilization. Generally, dispersions are prepared by incorporating the multipartite peptide into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active peptide plus any additional desired ingredient from a previously sterile-filtered solution thereof.

For administration by inhalation, the recombinant viruses are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

The present application is further illustrated by the following examples that should not be construed as limiting. The contents of all references, patents, and published patent applications cited throughout this application, as well as the Figures and Tables, are incorporated herein by reference.

### EXAMPLES

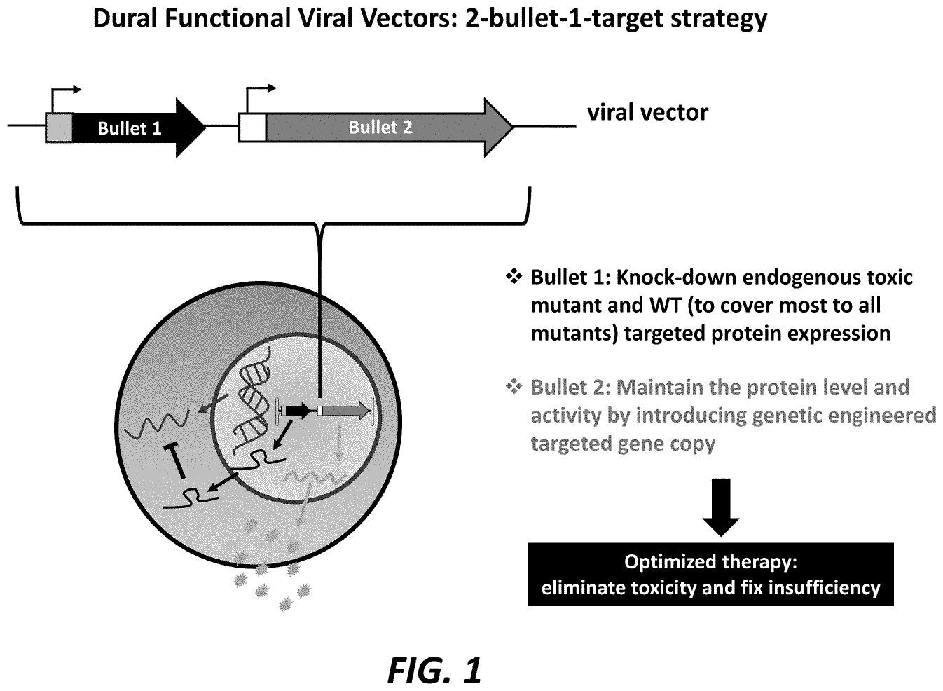

FIG. 1 shows a 2-bullet-1-target strategy of dual functional expression vector for treatment of single gene hereditary diseases caused by autosomal dominant negative, X linked dominant or X-linked recessive mutations. The dual functional vector comprises a nucleic acid suitably configured for expressing at least two active agents: (1) a first bullet “Bullet 1” expressing an siRNA via shRNA or miRNA sequences in the vector for knockdown or silencing of endogenous mRNA transcripts from targeted gene; and (2) a second bullet “Bullet 2” expressing a protein of the targeted gene being silenced that is insensitive to the silencing activity of siRNAs/miRNA. This dual functional vector are designed to achieve the optimized therapy by reducing endogenous toxic mutant as well as WT (to cover most to all mutants) gene expression of targeted protein and maintaining the protein level and activity by introducing genetic engineered targeted gene copy.

### Example 1. Two-Bullet-One-Target Strategy for Treatment of Hereditary Angioedema (HAE)

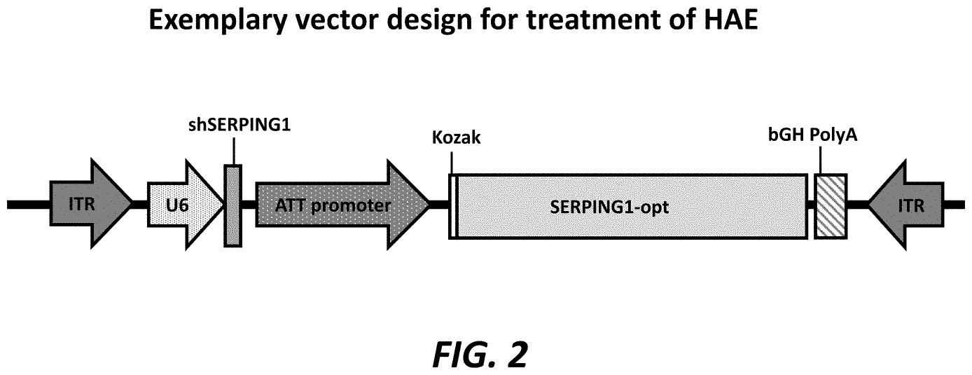

FIG. 2 shows an exemplary vector design for treatment of HAE. An AAV vector is employed which encodes an SERPING1 shRNA and a modified wild-type SERPING1 cDNA that is resistant to the inhibitory effect of the SERPING1 shRNA. Briefly, a single recombinant AAV vector gene therapy construct which uses shRNA designed to silence expression of the mutant and endogenous SERPING1 gene (Bullet 1), while simultaneously adding back a modified copy of SERPING1-opt (“Bullet 2”) cDNA expressing the wild-type version of SERPING1 protein to restore normal gene function. The cDNA sequences are optimized to remove restriction enzymes digestion sites, utilize alternative codons, and minimize CpGs. In the exemplary vector, the expression of the shRNA (“Bullet 1”) is under the control of a U6 promoter and the expression of the SERPING1-opt (“Bullet 2”) is under the control of a liver-specific ATT promoter. Several constructs, each containing different expression regulatory elements or different orientations or positions of “Bullet 1” and “Bullet 2” are developed as illustrated in FIG. 2.

### Example 2. In Vitro Screening of Two Bullets for Treatment of HAE

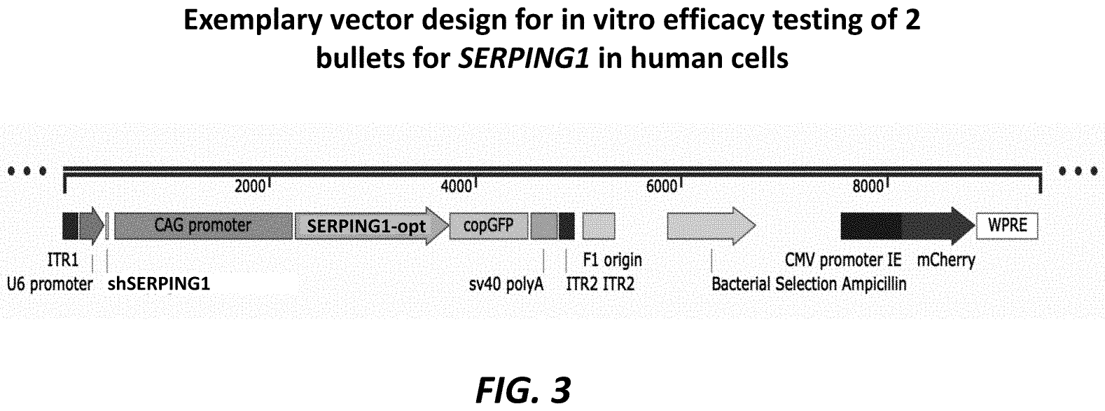

FIG. 3 shows an exemplary vector design for in vitro screening for the efficacy of 2 bullets for SERPING1 in human cell lines. A shRNA targeting human SERPING1 (shSERPING1) is expressed under U6 promoter. A codon optimized cDNA sequence (SERPING1-opt) with or without sequence coding for signal peptide is tagged with e.g., GFP reporters and expressed by CAG promoter. Several constructs, each containing different expression regulatory elements or different orientations or positions of “Bullet 1” and “Bullet 2” are developed as illustrated in FIG. 3. Expression of mCherry marker is served as transfection efficiency control of the vector. These constructs are screened for the efficiency of knockdown of endogenous SEPRING1 expression by shSERPING1 and the efficiency of expression and function of wild-type SERPING1 protein by genetically engineered codon optimized SERPING1-opt. The best candidate sequences of Bullet 1 and 2 are cloned into recombinant AAV vector construct for targeting SERPING1 mutations in the treatment of HAE as illustrated in FIG. 2.

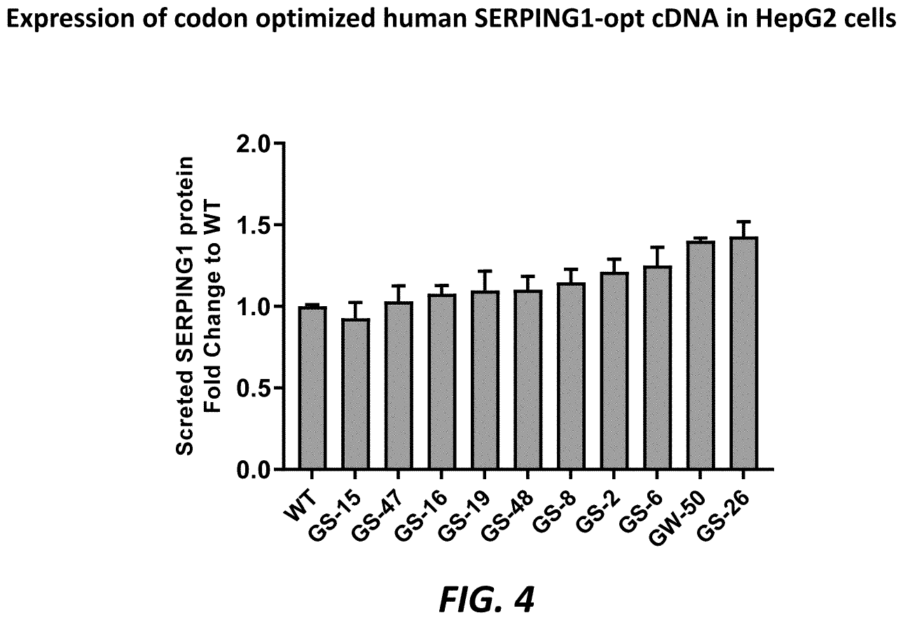

FIG. 4 shows the results of the expression of codon optimized human SERPING1-opt cDNA in human cells. Codon optimized SERPING1-opt (“Bullet 2”) cDNAs are designed to remove restriction enzymes digestion sites, utilize alternative codons, and minimize CpGs, in order to have increased and sustained expression level. These optimized sequences are cloned into expression plasmid (e.g., SEQ ID NO:1) and transfected into HepG2 cells. The expression of human SERPING1 protein (C1 inhibitor, C1-INH) is monitored and quantified by human specific SERPING1 protein ELISA (Sino Biological) in the culture medium and presented as the fold-change to WT protein expressed from SERPING1 cDNA (e.g., SEQ ID NO:3). Some engineered SERPING1-opt cDNAs have a higher expression level than the WT cDNA in HepG2 cells. The best candidate sequences of SERPING1-opt (“Bullet 2”) are further tested in vivo in mouse, then cloned into 2-bullet-1-target vector and recombinant AAV vector construct for validation. The nucleotide sequences of the relevant codon optimized human SERPING1-opt cDNAs are listed as SEQ ID NOS:4-14.

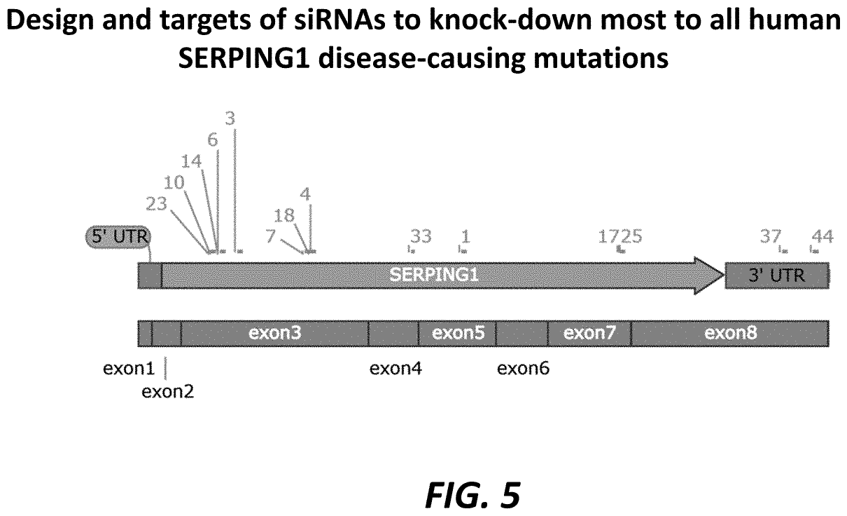

FIG. 5 shows the design and targets of siRNAs to knock-down most to all human SERPING1 disease-causing mutations. The siRNA ID number above SERPING1 cDNA diagram represent the targeted locations of the siRNAs. While the most siRNAs are designed to target the open reading frame of SERPING1 excluding the disease-causing mutant nucleotides, some target the 3′UTR. These siRNAs are expected to knockdown all human SERPING1 disease-causing mutant as well as the endogenous WT transcripts. The nucleotide sequences of the relevant human SERPING1 siRNAs are listed as SEQ ID NOS:15-22.

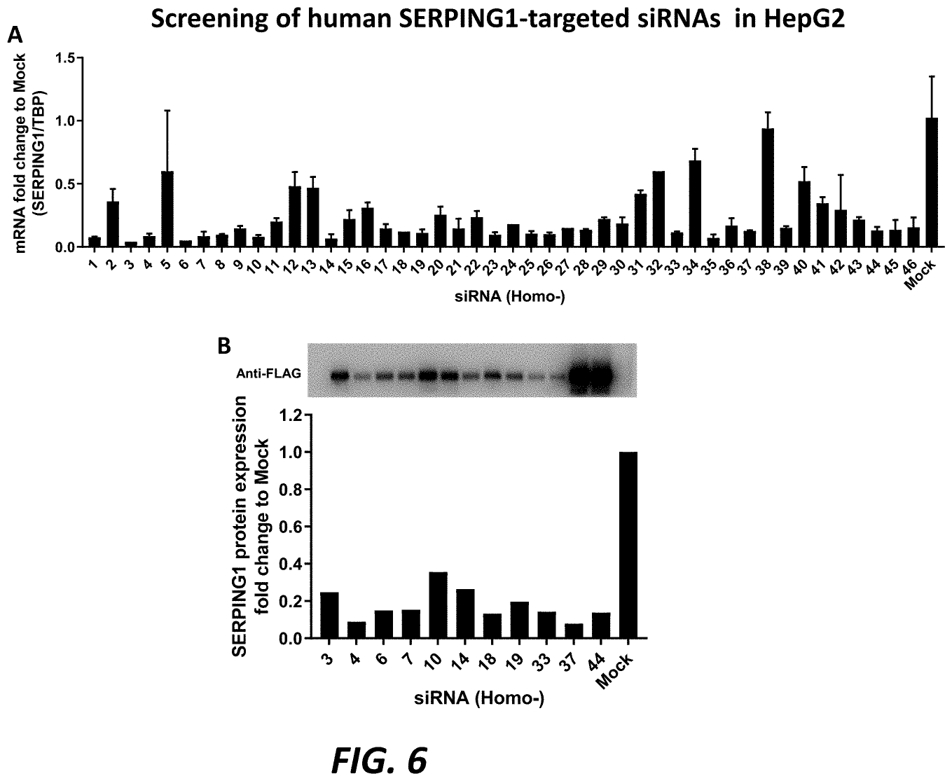

FIG. 6, panels A-B show the results of screening of human SERPING1-targeted siRNAs for the knock-down effect on SERPING1 expression. Effect on endogenous SERPING1 mRNA is assayed in HepG2 cells by transfecting siRNA at concentration of 1 nM with RNAiMAX in the cell culture medium. Alternatively, siRNA sequences are cloned into plasmid (e.g., SEQ ID NO:2) and transfected into HepG2. Cells are collected after 24 h and cell lysates are subjected to qPCR (TaKaRa) to monitor mRNA level of SERPING1 and TBP (internal control). SERPING1 mRNA level is normalized to TBP and calculated as the fold change to the mock transfection control (FIG. 6, panel A). Effect on SERPING1 protein expression is assessed by Western-blot (WB). A plasmid expressing WT human SERPING1 with FLAG tag and siRNA are co-transfected into HEK-293 cells using Lipofectamine 3000. Cells are collected after 2d and lysed. SERPING1 protein expression level is monitored by anti-FLAG WB, quantified and calculated as the fold change to the mock siRNA transfection control (FIG. 6, panel B). Most siRNAs targeting human SERPING1 tested in the screening cause up to 90% reduction of human SERPING1 mRNA and protein level.

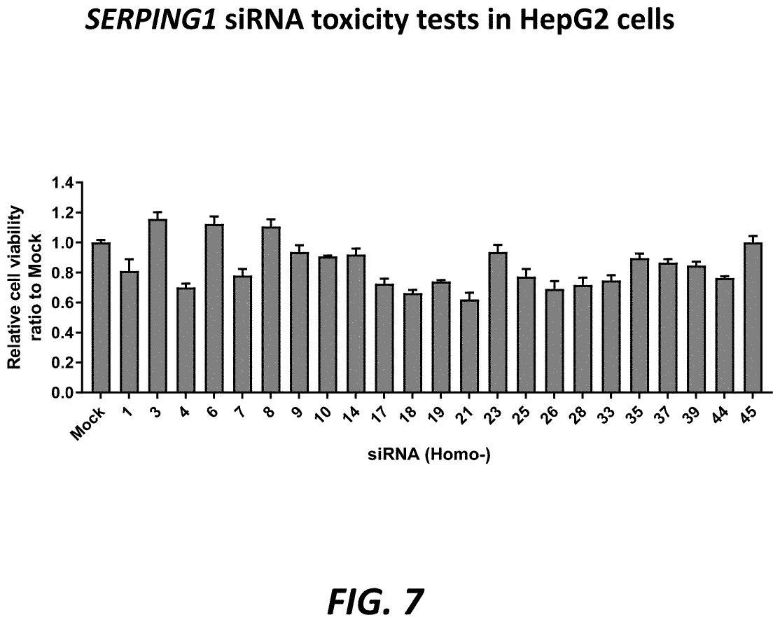

FIG. 7 shows the results of SERPING1 siRNA toxicity tests by cell proliferation in human cells. Cell proliferation rate is assessed by CCK8 assay (Dojindo) in siRNA at final concentration of 50 nM transfected HepG2 after 3d using RNAiMAX. The relative cell viability is calculated as the ratio to mock siRNA transfection. Most siRNAs tested do not have a toxicity effect on HepG2 cells as there is no remarkable decrease in cell viability compared to the mock transfection. The best candidate sequences of siRNA targeting human SERPING1 (“Bullet 1”) with high knock-down efficiency and low cytotoxicity are cloned into 2-bullet-1-target vector and recombinant AAV vector construct for further testing.

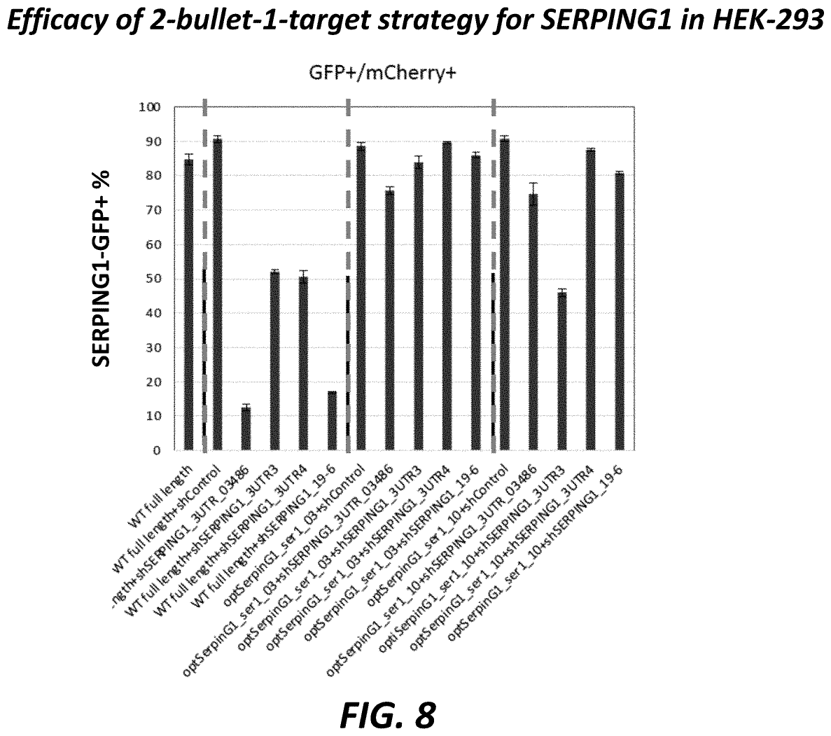

FIG. 8 shows the results of efficacy of 2-bullet-1-target strategy for SERPING1 in human cells. The best candidate sequences of SERPING1 siRNA are converted to shRNA. shRNA (“Bullet 1”) and WT SERPING1 or codon optimized SERPING1-opt cDNA without sequence coding for signal peptide (“Bullet 2”) are cloned into 2-bullet-1-target vector on plasmid as illustrated in FIG. 3. HEK-293 cells transfected with the plasmid are sorted by flow cytometry and gated for GFP (SERPING1-GFP protein expression) and mCherry (transfected cell control). Percentage of GFP+ cells are quantified in mCherry+ cells. The WT SERPING1 expression are reduced by shRNA (e.g., shSERPING1_3UTR_03486, shSERPING1_3UTR, shSERPING1_4UTR, shSERPING1_19-6) with various efficiency compared to control shRNA (shControl). In most cases, the expression of codon optimized SERPING-opt cDNA (e.g., optSerpinG1_ser1_03, optSerpinG1_ser1_10) is not reduced by shRNA. The nucleotide sequences of the relevant human SERPING1 shRNAs are listed as SEQ ID NOS:23-39.

These results demonstrate that in human cell culture, shRNA targeting human SERPING1 effectively knockdown endogenous and non-engineered WT SERPING1 expression but do not reduce the engineered codon optimized SERPING1-opt expression. Thus, the dual functional vector comprises “2 bullets” can (1) reduce endogenous toxic mutant as well as the WT copy of targeted protein expression; and at the same time (2) maintain the protein level and activity by introducing genetic engineered targeted gene copy. This 2-bullet-1-target strategy provide the optimized therapy for HAE by eliminating the toxicity caused by mutant SEPRING1 protein which executes a dominant negative effect, and at the same time fixing the insufficiency of SEPRING1 protein activity.

### Example 3. In Vivo Testing of 2-Bullet-1-Target Strategy for Treatment of HAE Using Mouse Model

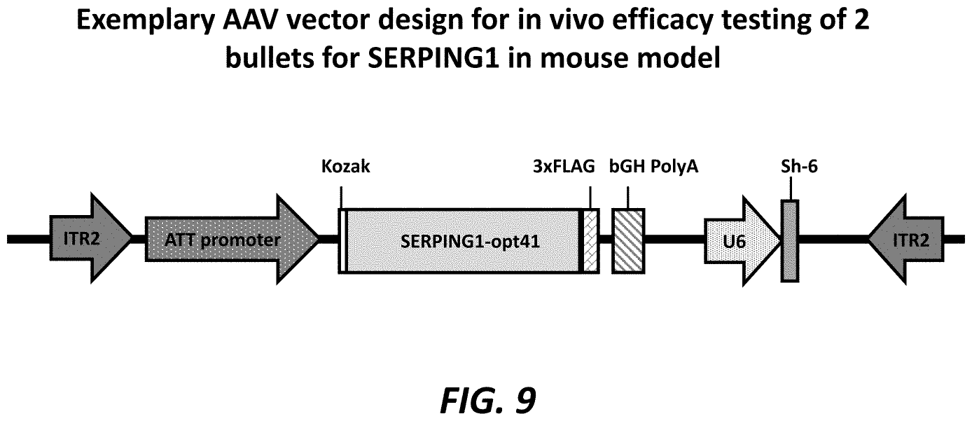

FIG. 9 shows an exemplary AAV vector design for in vivo efficacy testing of 2 bullets for SERPING1 in mouse model. A shRNA targeting mouse SERPING1 (e.g., Sh-6) is expressed under U6 promoter. A codon optimized human SERPING1-opt cDNA sequence (e.g., SERPING1-opt41) is tagged with e.g., FLAG and expressed under ATT promoter. Several constructs, each containing different expression regulatory elements or different orientations or positions of “Bullet 1” and “Bullet 2” are developed as illustrated in FIG. 9. These constructs are tested for the efficiency of knockdown of endogenous mouse SEPRING1 expression and the efficiency of expression and function of genetically engineered codon optimized human SERPING1-opt cDNA in mouse.

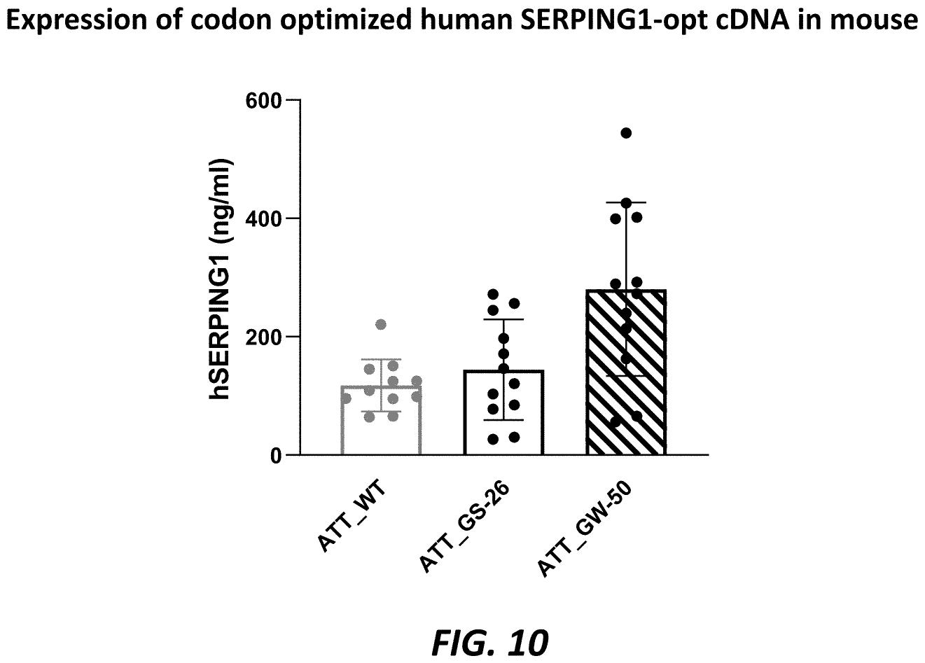

FIG. 10 shows the results of the expression of codon optimized human SERPING1-opt cDNA in mouse. Expression vector on plasmid (10 μg) containing WT SERPING1 or codon optimized SERPING1-opt cDNA sequences controlled under the liver-specific promoter ATT are injected intravenously through tail vein into WT C57BL/6 male mouse aged ˜6 W (n=11-12/group). Mouse sera are collected 3d after injection. The content of human SERPING1 (hSERPING1) protein in mouse serum is monitored and quantified by human specific SERPING1 ELISA (Sino Biological). In vivo, codon optimized SERPING1-opt GW-50 cDNA causes a more than 2-fold increase in expression level compared with WT.

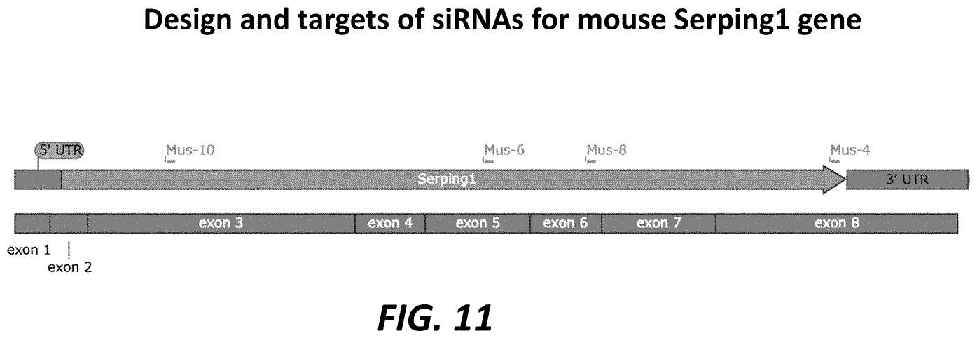

FIG. 11 shows the design and targets of siRNAs for mouse SERPING1 gene. In order to test the efficacy of 2-bullet-1-target strategy in mouse model, siRNAs targeting mouse SERPING1 (m SERPING1) transcripts are designed and screened. The ID numbers of siRNA above m SERPING1 cDNA diagram represent the targeted locations. These siRNAs are expected to knockdown endogenous m SERPING1 expression. The nucleotide sequences of the relevant mouse SERPING1 siRNA are listed as SEQ ID40-43.

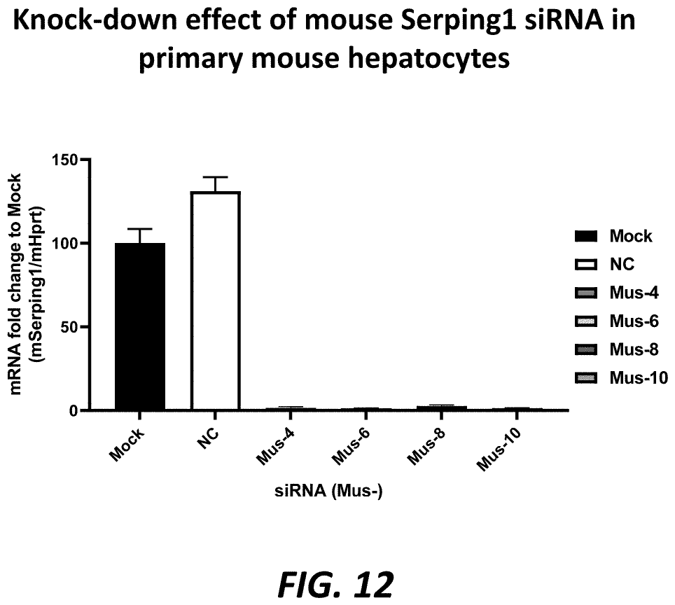

FIG. 12 shows the results of knock-down effect of mouse SERPING1 siRNA in primary mouse hepatocytes. Cells are transfected by siRNA targeted to mSERPING1 or negative control (NC) siRNA at a concentration of 20 nM with RNAiMAX in the cell culture medium. Cells are collected after 24 h and cell lysates are subjected to qPCR to monitor mRNA level of mouse SERPING1 and Hprt (internal control). mSERPING1 mRNA level is normalized to mHprt and calculated as the fold change (Fc) to the mock transfection control. All the siRNAs tested significantly reduce the endogenous mSERPING1 expression to 1-2%, while NC siRNA has no knock-down effect.

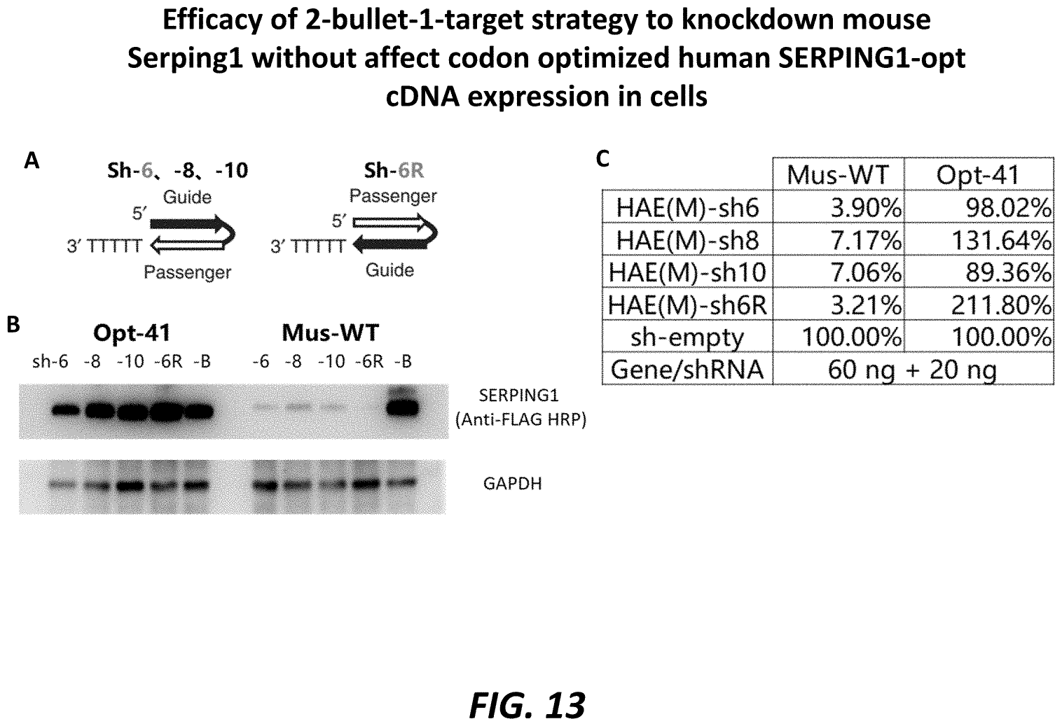

FIG. 13, panels A-C show the results of efficacy of 2-bullet-1-target strategy to knockdown mouse SERPING1 without affecting codon optimized human SERPING1-opt cDNA expression in cells. Mouse SERPING1-targeted siRNA (Mus-6, 8 or 10) is converted into shRNA (Sh-6, -8, -10, or -6R). Passenger strand (sense sequence) and guide strand (anti-sense sequence) are connected by a loop sequence with a 3′TTTTT overhang. Passenger strand comes first shown as Sh-6 or guide stand comes first shown as Sh-6R (FIG. 13, panel A). Expression plasmid containing mouse shRNA (e.g., sh-6, -8, -10, -6R) (20 ng) and plasmid containing codon optimized human SERPING1-opt cDNA (e.g., Opt-41) or mouse SERPING1 cDNA tagged with FLAG (60 ng) are co-transfected into HEK-293 cells cultured in 96 w plate with Lipofectamine 3000. Cells are collected 3d after transfection and the expression of mouse SERPING1 (Mus-WT) and human SERPING1 (Opt-41) is monitored by Western-blot with FLAG (FIG. 13, panel B) and knock-down efficiency to mouse or human SERPING1 protein is quantified (FIG. 13, panel C). Mouse shRNAs efficiently knockdown the mouse SERPING1 (Mus-WT) expression to less than 8% while do not reduce human SERPING1 protein level expressed by codon optimized cDNA (Opt-41). The nucleotide sequences of the relevant mouse NO:1 shRNA are listed as SEQ ID44-47.

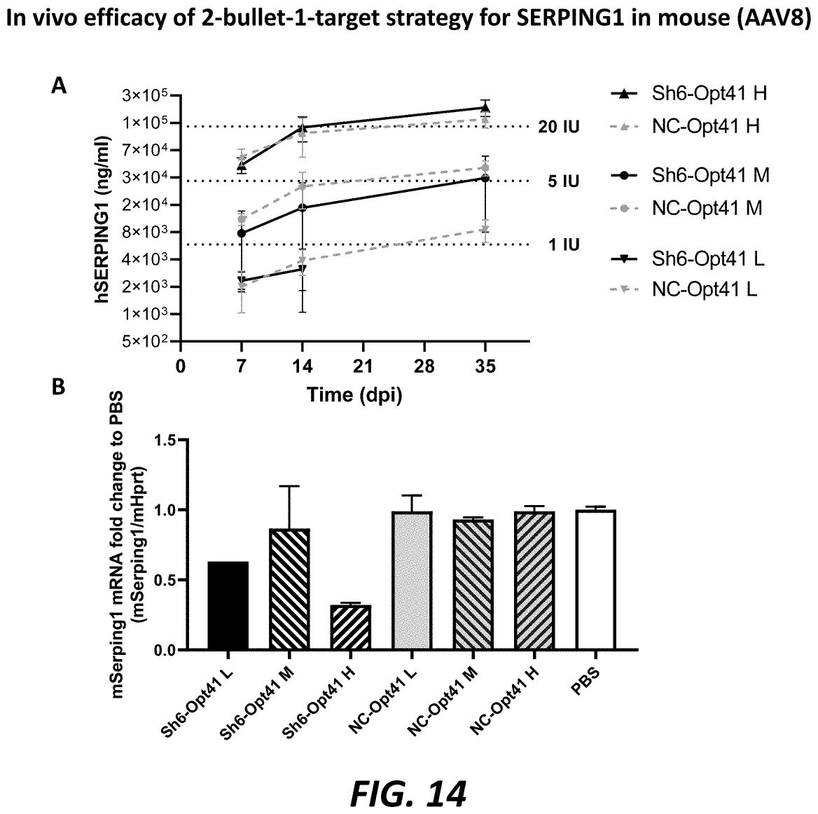

FIG. 14, panels A-B show the results of in vivo efficacy of 2-bullet-i-target strategy for SERPING1 in mouse. Dual function AAV vector are constructed as shown in FIG. 9. An shRNA targeting mouse SERPING1 (Sh6) or negative control shRNA (NC) is expressed under U6 promoter. A codon optimized human SERPING1-opt cDNA sequence (Opt41) is expressed under ATT promoter. The AAV8 vector is produced by triple-plasmid system in HEK-293 cells and purified by anion exchange chromatography. Vector genome titers are determined by quantitative TaqMan real-time PCR. C57BL/6 male mice aged ˜6 w (n=4/group) are injected intravenously through tail vein in 100 μl at low (L), medium (M) or high (H) dose of 5×10¹¹, 2×10¹², 8×10¹² vg/kg, respectively. On 7, 14, 35 d post injection (dpi) mouse sera are collected and human SERPING1 protein level are measured by ELISA in mouse plasma (FIG. 14, panel A). On 35 dpi, mRNA level of mouse SERPING1 and Hprt is measured in mouse liver lysates. mSERPING1 mRNA level is normalized to mHprt and calculated as the fold change to the PBS control (FIG. 14, panel B). These results demonstrate that the dual function AAV8 vector generate high level of human SERPING1 protein expression in mouse plasma at a dose dependent manner. At a high (H) dose of 8×10¹² vg/kg, secreted hSERPING1 protein reaches ˜20 IU in mouse serum on 35 dpi (Sh6-Op41 H and NC-Opt41 H). Meanwhile, the dual function AAV8 vector with shRNA targeting mSERPING1 knock-down the endogenous mSERPING1 expression. At the high dose (H), Sh6-Opt41 reduces mSERPING1 expression to ˜30%, while the AAV8 vector NC-Opt41 containing negative control shRNA does not decrease mSERPING1 expression.

Taking together, these results demonstrate that in vivo, the dual function vector of 2-bullet-1-target strategy efficiently knockdown endogenous mSERPING1 expression and express genetically engineered codon optimized hSERPING1-opt cDNA in mouse. The 2 bullet-i-target dual functional vector as illustrated in FIG. 2 can be used to achieve the optimized therapy for HAE caused by dominant negative mutations of SERPING1 by reducing endogenous toxic mutant as well as WT (to cover all mutants) gene expression and maintaining the protein level and activity by introducing genetic engineered codon optimized copy.

### Example 4. Two-Bullet-One-Target Strategy for Treatment of Amyotrophic Lateral Sclerosis (ALS)

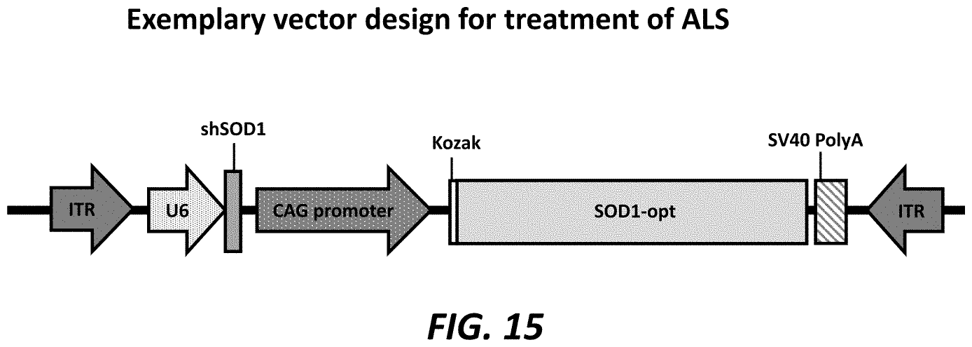

In another aspect, the present application further provides compositions and methods for treatment of ALS. FIG. 15 shows a 2-bullet-i-target strategy specifically designed for the treatment of familial amyotrophic lateral sclerosis (ALS), which is analogous to the approach described above for treatment of HAE.

FIG. 15 shows an exemplary vector design for treatment of ALS. An AAV vector is employed which encodes an SOD1 shRNA (“Bullet 1”) under U6 promoter, and a modified codon optimized SOD1-opt cDNA (“Bullet 2”) under CAG promoter that is resistant to the inhibitory effect of the SERPING1 shRNA to restore normal gene function. Several constructs, each containing different expression regulatory elements or different orientations or positions of “Bullet 1” and “Bullet 2” are developed as illustrated in FIG. 15.

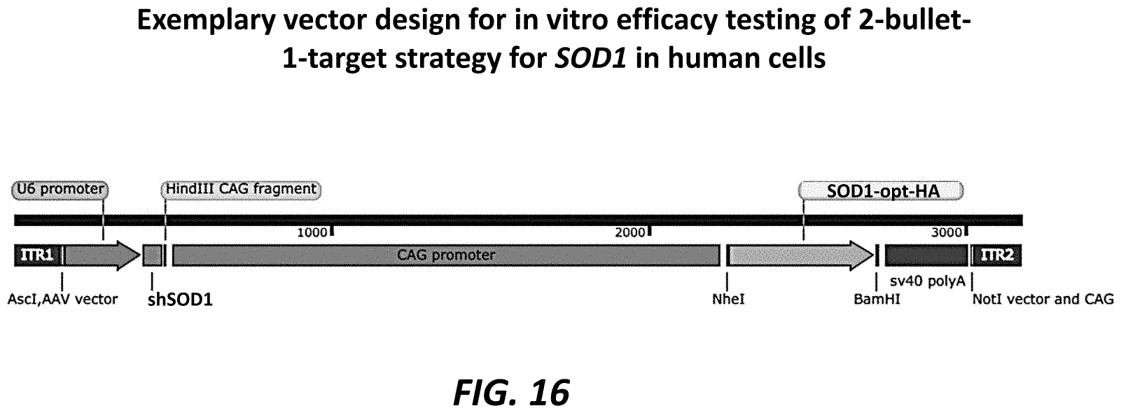

FIG. 16 shows an exemplary vector design for in vitro screening for the efficacy of 2-bullet-1-target for SOD1 in human cell lines. An shRNA converted from siRNA targeting human SOD1 (ShSOD1) is expressed under U6 promoter. A codon optimized SOD1-opt cDNA sequence is tagged with e.g., HA reporter and expressed by CAG promoter. These constructs are screened for the efficiency of knockdown of endogenous SOD1 expression by shRNA and the efficiency of expression and function of wild-type SOD1 protein by genetically engineered codon optimized SOD1-opt cDNA. Several constructs, each containing different expression regulatory elements or different orientations or positions of “Bullet 1” and “Bullet 2” are developed as illustrated in FIG. 16. The best candidate sequences of Bullet 1 and 2 are cloned into recombinant AAV vector construct for targeting SOD1 mutations in the treatment of ALS as illustrated in FIG. 15. The nucleotide sequences of the relevant human SOD1 siRNA are listed as SEQ ID NOS:48-57. The nucleotide sequences of the relevant human SOD1 shRNA are listed as SEQ ID NOS:58-67. The nucleotide sequences of the relevant human SOD1-opt cDNA are listed as SEQ ID NOS:68-73.

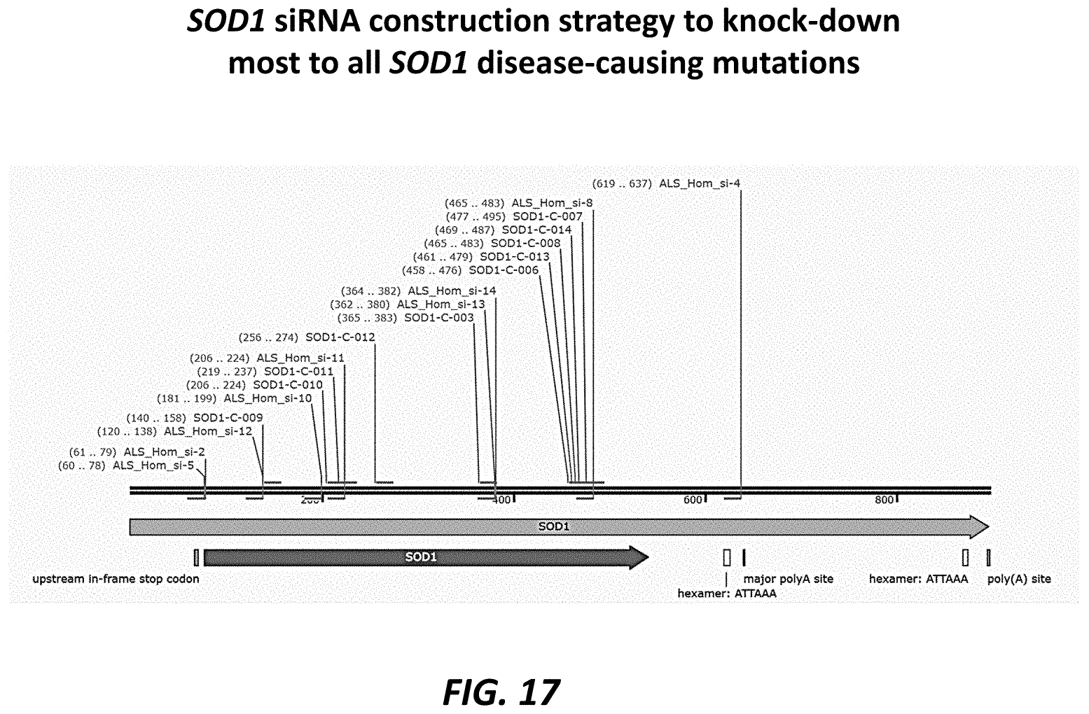

FIG. 17 shows SOD1 shRNA construction strategy to knock-down most to all SOD1 disease-causing mutations. The labels above SOD1 cDNA diagram represent the targeted locations of the siRNAs. While the most siRNAs are designed to target the open reading frame of SOD1 excluding the disease-causing mutant nucleotides, some target the upstream or downstream region.

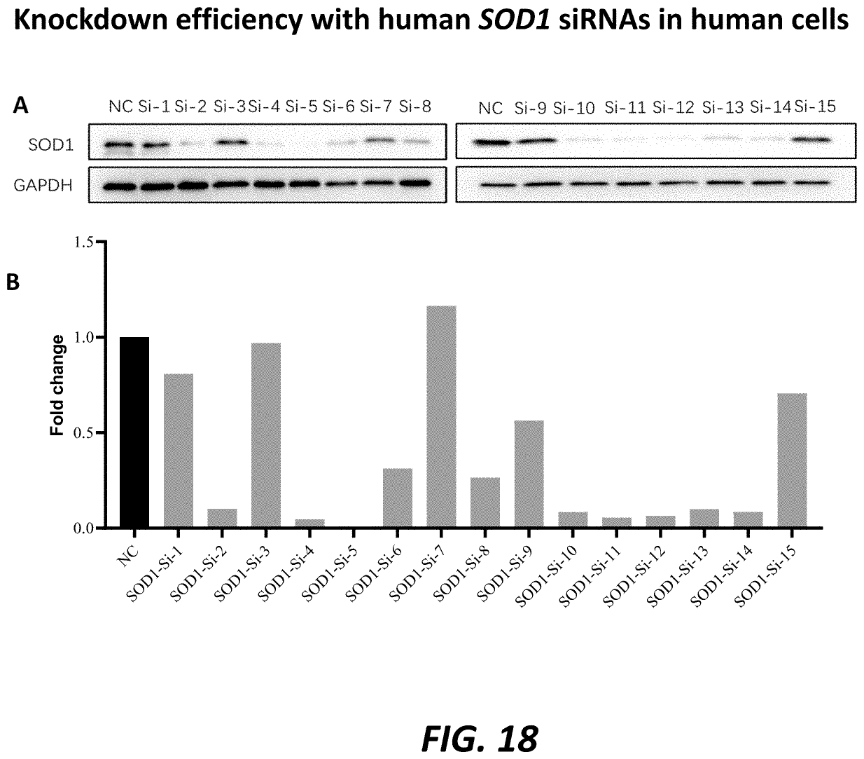

FIG. 18, panels A-B show the results of screening of human SOD1-targeted siRNA in human cells. The exemplary Western blot (FIG. 18, panel A) and quantification (FIG. 18, panel B) to detect the knock-down efficiency on endogenous SOD1 expression by siRNA. Several siRNAs targeting human SOD1 reduce more than 95% of human SOD1 protein level, while the negative control (NC) siRNA has no effect. The best candidate sequences of siRNA targeting human SOD1 (“Bullet 1”) with high knock-down efficiency are further tested in vivo.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the embodiments and claims described herein. While various embodiments have been described above, it should be understood that such disclosures have been presented by way of example only and are not limiting. Thus, the breadth and scope of the subject compositions and methods should not be limited by any of the above-described exemplary embodiments and their equivalents. The embodiments and claims are intended to cover the components and steps in any sequence which is effective to meet the intended objectives, unless the context specifically indicates the contrary.

What is claimed is:

1. An expression vector, comprising:
a first nucleic acid sequence suitably configured for expressing a first active agent, wherein the first active agent comprises an siRNA, shRNA or miRNA that has silencing activity for silencing mRNA transcripts expressed from one or more mutant alleles of a gene, and wherein the one or more mutant alleles of the gene are associated with a disease in a patient; and
a second nucleic acid sequence suitably configured for expressing a second active agent, wherein the second active agent is a polypeptide corresponding to a wild-type allele of the gene, and wherein the second nucleic acid sequence is genetically engineered to produce an mRNA transcript for the second active agent that is insensitive to the silencing activity of the first active agent.

2. The expression vector of claim 1, wherein the disease is a genetic disease caused by one or more autosomal dominant mutations, one or more X-linked dominant mutations or one or more X-linked recessive mutations.

3. The expression vector of claim 1, wherein the disease is listed in Tables 1-3 and wherein the first and second active agents correspond to a gene associated with a disease in any one of Tables 1-3.

4. The expression vector of claim 1, wherein the disease is hereditary angioedema (HAE), wherein the first active agent is targeted for silencing the endogenous C1 esterase inhibitor transcripts, including mutant and wild-type, and wherein the second active agent is a wild type C1 esterase inhibitor polypeptide.

5. The expression vector of claim 4, wherein the first active agent comprises a nucleotide sequence selected from the group consisting of SEQ ID NOs:15-22.

6. The expression vector of claim 5, wherein the first active agent comprises the nucleotide sequence of SEQ ID NO:15.

7. The expression vector of claim 4, wherein the first active agent comprises a nucleotide sequence selected from the group consisting of SEQ ID NOs:23-39.

8. The expression vector of claim 7, wherein the first active agent comprises the nucleotide sequence of SEQ ID NO:23.

9. The expression vector of claim 1, wherein the disease is familial amyotrophic lateral sclerosis (fALS), wherein the first active agent is targeted for silencing the endogenous superoxide dismutase 1 (SOD1) mRNA transcripts, including mutant and wild-type, and wherein the second active agent is a wild type SOD1 polypeptide.

10. The expression vector of claim 9, wherein the first active agent comprises a nucleotide sequence selected from the group consisting of SEQ ID NOs:48-57.

11. The expression vector of claim 10, wherein the first active agent comprises a nucleotide sequence of SEQ ID NO:48.

12. The expression vector of claim 9, wherein the first active agent comprises a nucleotide sequence selected from the group consisting of SEQ ID NOs:58-67.

13. The expression vector of claim 12, wherein the first active agent comprises the nucleotide sequence of SEQ ID NO:58.

14. The expression vector of claim 1, wherein the expression vector is a recombinant virus vector derived from adenovirus-associated virus (AAV), retrovirus, lentivirus or adenovirus.

15. The expression vector of claim 1, wherein the expression vector is a recombinant AAV vector.

16. The expression vector of claim 15, wherein the first active agent comprises a nucleotide sequence selected from the group consisting of SEQ ID NOs:15-39.

17. The expression vector of claim 15, wherein the first active agent comprises a nucleotide sequence selected from the group consisting of SEQ ID NOs:48-67.

18. A pharmaceutical composition comprising:
the expression vector of claim 1; and
a pharmaceutically acceptable carrier.

19. A method for treating HAE, comprising:
administering an effective amount of the expression vector of claim 4 into a subject in need of such treatment.

20. A method for treating fALS, comprising:
administering an effective amount of the expression vector of claim 9 into a subject in need of such treatment.
